(19) United States
(12) Patent Application Publication
(10) Pub. No.: US 2026/0259218 A1
(43) Pub. Date: Sep. 3, 2026

(54) METHODS AND SYSTEMS FOR NANOPORE SEQUENCING OF MODIFIED AMINO ACIDS

(71) Applicant: Glyphic Biotechnologies, Inc., New York, NY (US)

(72) Inventors: Joshua Young Cynming YANG, Richardson, TX (US); Eugene TU, Portland, OR (US); Steven Arnold SUNDBERG, San Francisco, CA (US); Asmamaw WASSIE, New York, NY (US); Daniel Masao ESTANDIAN, New York, NY (US); Andrew John PRICE, New York, NY (US); Chao Bai HUANG, New York, NY (US); Jai PRAKASH, New York, NY (US); Alexander Julian TRAN, New York, NY (US); Sareh MORADI FARD, New York, NY (US); Christian SUPRIANO, New York, NY (US); Colin M. WARNES, Jr., New York, NY (US); Pol ARRANZ-GIBERT, New York, NY (US); Yinghua SUN, New York, NY (US); Norman LUONG, New York, NY (US); Chih-Kai LIAO, New York, NY (US); Gerardo Fabian DELGADO, New York, NY (US); Hongye SUN, New York, NY (US)

(21) Appl. No.: 19/676,652

(22) Filed: May 13, 2026

Related U.S. Application Data
(63) Continuation-in-part of application No. 19/662,044, filed on Apr. 29, 2026.
Application No. 19/662,044 is a continuation of application No. PCT/US25/13852, filed on Jan. 30, 2025.
(60) Provisional application No. 63/627,214, filed on Jan. 31, 2024.
(60) Provisional application No. 63/558,344, filed on Feb. 27, 2024.
(60) Provisional application No. 63/683,941, filed on Aug. 16, 2024.
(60) Provisional application No. 63/694,299, filed on Sep. 13, 2024.
(60) Provisional application No. 64/027,386, filed on Apr. 3, 2026.

(51) Int. Cl. G01N 33/68; G01N 33/487

(52) U.S. Cl. CPC G01N 33/6818; G01N 33/48721

(57) ABSTRACT

Provided herein are methods and systems for nanopore-based identification and sequencing of modified amino acids. In some aspects, a method for processing a modified amino acid comprises providing the modified amino acid comprising a polymerizable molecule and a plurality of monomers; incorporating a monomer into or adjacent to the polymerizable molecule to generate a partially polymerized modified amino acid; and translocating the partially polymerized modified amino acid through a nanopore. In some embodiments, signals measured at or adjacent to the nanopore under one or more applied voltages are used to identify amino acid types.

### CROSS-REFERENCE

This application is a continuation-in-part of U.S. application Ser. No. 19/662,044, filed Apr. 29, 2026, which is a continuation of International Application No. PCT/US2025/013852, filed Jan. 30, 2025, which claims the benefit of U.S. Provisional Application Nos. 63/627,214, filed Jan. 31, 2024, 63/558,344, filed Feb. 27, 2024, 63/683,941, filed Aug. 16, 2024, and 63/694,299, filed Sep. 13, 2024; and U.S. patent application Ser. No. 19/662,044, filed Apr. 29, 2026, claims the benefit of U.S. Provisional Application No. 64/027,386, filed Apr. 3, 2026, each of which is incorporated by reference herein in its entirety.

### SEQUENCE LISTING

The instant application contains a Sequence Listing which has been filed electronically in XML format and is hereby incorporated by reference in its entirety. Said XML copy, created May 14, 2026 is named 60652-718.501SL.xml and is 2,519 bytes in size.

### BACKGROUND

Technological improvements in the analysis and characterization of biological molecules have proven to be critical in understanding biological and pathological mechanisms, which have implications in disease diagnosis and modeling, development of therapeutics and treatment, and improving health outcomes. Among these technological improvements, nucleic acid sequencing has emerged as an important tool for genomic and transcriptomic analysis of biological samples.

Protein signaling underpins a variety of cellular processes and serves important functions in viruses, cells, and living organisms. However, current technologies for studying proteins are limited in selectivity, sensitivity, throughput, or require a priori knowledge.

### SUMMARY

There is a need for improved methods of single-molecule protein and peptide sequencing, and in particular for methods and systems capable of identifying individual amino acids at the single-molecule level with high accuracy. The methods and systems presented herein address the abovementioned need. A brief summary of various exemplary embodiments is presented. Some simplifications and omissions may be made in the following summary, which is intended to highlight and introduce aspects of certain embodiments disclosed herein, but not to limit the scope of the disclosure. Detailed descriptions of various embodiments adequate to allow those of ordinary skill in the art to make and use the concepts disclosed herein will follow in later sections.

In some aspects of the present disclosure, provided herein is a method for processing a modified amino acid, comprising: (a) providing the modified amino acid and a plurality of monomers, wherein the modified amino acid comprises a polymerizable molecule; (b) incorporating a monomer of the plurality of monomers into or adjacent to the polymerizable molecule, thereby generating a partially polymerized modified amino acid; and (c) translocating the partially polymerized modified amino acid or a portion thereof through a nanopore.

In some embodiments, the method further comprises (d), measuring a signal of or adjacent to the nanopore, thereby generating a measured signal. In some embodiments, (d) occurs during (c). In some embodiments, the method further comprises (e), using the measured signal or a metric or characteristic thereof to determine an amino acid type of the modified amino acid. In some embodiments, the measured signal is a current signal under an applied voltage.

In some embodiments, the method further comprises measuring a plurality of signals of or adjacent to the nanopore, thereby generating a plurality of measured signals. In some embodiments, the plurality of measured signals is a plurality of current signals under a plurality of applied voltages or a time-varying voltage. In some embodiments, the method further comprises using the plurality of measured signals or a metric or characteristic thereof to identify an amino acid type of the modified amino acid.

In some embodiments, (c) occurs in a cis-to-trans direction. In some embodiments, (c) is performed by applying an electric potential to the nanopore. In some embodiments, the method further comprises reversing the electric potential, thereby translocating the partially polymerized modified amino acid in a trans-to-cis direction. In some embodiments, the method further comprises repeating (b)-(c) and the reversing. In some embodiments, the method further comprises (f) measuring an additional plurality of signals of or adjacent to the nanopore during the repeating. In some embodiments, the method further comprises using the additional plurality of signals or a metric or characteristic thereof to identify an amino acid type of the modified amino acid.

In some embodiments, the metric or characteristic thereof is a mean value (μ_{signal}), a median value, a range of values (e.g., max signal minus min signal), a standard deviation or noise of the signal (σ_{signal}), a coefficient of variation (σ_{signal}/μ_{signal}×100), a signal-to-noise ratio (μ_{signal}/σ_{signal}), peak width, peak signal, full width half max, peak position, separation resolution, area-under-the-curve (AUC), or combination thereof. In some embodiments, the metric or characteristic thereof is a conductance or a conductance rate of change.

In some embodiments, the polymerizable molecule comprises a nucleic acid molecule. In some embodiments, the nucleic acid molecule is at least partially single-stranded. In some embodiments, prior to (b), the nucleic acid molecule comprises a double-stranded priming region. In some embodiments, the plurality of monomers comprises a plurality of nucleotides, and the monomer is incorporated adjacent to the double-stranded priming region. In some embodiments, (b) is performed using a polymerizing enzyme. In some embodiments, the polymerizing enzyme is a polymerase. In some embodiments, the monomer is a nucleotide or modified nucleotide, and, during (b), the polymerase incorporates a single nucleotide or modified nucleotide to the nucleic acid molecule. In some embodiments, the nucleic acid molecule comprises an error correction barcode sequence. In some embodiments, the error correction barcode sequence comprises a Hamming code, a Levenshtein code, or a Reed-Solomon code.

In some embodiments, the modified amino acid is comprised by a stacked plurality of modified amino acids. In some embodiments, the stacked plurality of modified amino acids comprises a first end and a second end, wherein the first end or the second end comprises a blocking region, wherein the blocking region prevents the stacked plurality of modified amino acids from escaping the nanopore. In some embodiments, the first end and the second end comprise the blocking region. In some embodiments, the blocking region comprises a hairpin structure, a G-quadruplex, or a protein.

In some embodiments, (b) and (c) are performed in a single reaction mixture. In some embodiments, the method further comprises generating the modified amino acid, wherein the generating comprises (I) providing a linker and a polymerizable molecule, (II) coupling the linker to (i) an amino acid of a peptide and (ii) the polymerizable molecule to generate an amino acid-linker complex, and (III) cleaving the amino acid, thereby generating the modified amino acid, wherein the modified amino acid comprises a cleaved amino acid, the linker, and the polymerizable molecule. In some embodiments, the coupling of the linker to the polymerizable molecule occurs prior to (I).

In some embodiments, the method further comprises re-sequencing the modified amino acid, wherein the re-sequencing comprises removing the monomer from the partially polymerized modified amino acid, thereby re-generating the modified amino acid and repeating (b)-(c). In some embodiments, the re-sequencing further comprises measuring an additional signal of or adjacent to the nanopore, thereby generating an additional measured signal. In some embodiments, the method further comprises using the measured signal and the additional measured signal to identify an amino acid type, wherein an identification accuracy of the amino acid type is higher using the measured signal and the additional measured signal as compared to an identification accuracy using only the measured signal or only the additional measured signal. In some embodiments, the method further comprises performing the re-sequencing at least 5 times, wherein a consensus accuracy of the re-sequencing for a given amino acid type exceeds 80% for at least 10 distinct amino acid types.

In some embodiments, the nanopore is an MspA nanopore or variant thereof.

In some embodiments, (a) further comprises providing a plurality of modified amino acids including the modified amino acid. In some embodiments, the method further comprises performing differential labeling of the plurality of modified amino acids to confirm the identity of at least one modified amino acid of the plurality of modified amino acids.

In some aspects of the present disclosure, provided herein is a method for identifying a stacked plurality of modified amino acids, comprising: (a) providing the stacked plurality of modified amino acids, wherein the stacked plurality of modified amino acids comprises a plurality of modified amino acids comprising a plurality of polymerizable molecules, wherein the plurality of polymerizable molecules are coupled to one another in a linear fashion; (b) contacting the stacked plurality of modified amino acids with a plurality of monomers; (c) incorporating a monomer of the plurality of monomers into or adjacent to a polymerizable molecule of the plurality of polymerizable molecules, thereby generating a partially polymerized stacked plurality of modified amino acids; (d) translocating, in a first direction, the partially polymerized stacked plurality of modified amino acids or a portion thereof through a nanopore; (e) translocating, in a second direction, the partially polymerized stacked plurality of modified amino acids or a portion thereof through the nanopore; and (f) repeating (c)-(e) on the partially polymerized stacked plurality of modified amino acids.

In some embodiments, the method further comprises, during (d) or (e), measuring a signal from or adjacent to the nanopore, thereby generating a measured signal. In some embodiments, the method further comprises using the measured signal or a metric or characteristic thereof to determine an amino acid type of a modified amino acid of the stacked plurality of modified amino acids. In some embodiments, the measured signal is a current signal under an applied voltage. In some embodiments, the method further comprises measuring a plurality of signals of or adjacent to the nanopore, thereby generating a plurality of measured signals. In some embodiments, the plurality of measured signals is a plurality of current signals under a plurality of applied voltages or a time-varying voltage.

In some embodiments, the method further comprises measuring an additional plurality of signals of or adjacent to the nanopore during (f). In some embodiments, the method further comprises using the additional plurality of signals or a metric or characteristic thereof to identify an amino acid type of the stacked plurality of modified amino acids. In some embodiments, the method further comprises using the plurality of measured signals or a metric or characteristic thereof to identify one or more amino acid types of the plurality of modified amino acids. In some embodiments, the metric or characteristic thereof is a mean value (μsignal), a median value, a range of values (e.g., max signal minus min signal), a standard deviation or noise of the signal (σsignal), a coefficient of variation (σsignal/μsignal×100), a signal-to-noise ratio (μsignal/σsignal), peak width, peak signal, full width half max, peak position, separation resolution, area-under-the-curve (AUC), or combination thereof. In some embodiments, the metric or characteristic thereof is a conductance or a conductance rate of change.

In some embodiments, the plurality of polymerizable molecules comprises a plurality of nucleic acid molecules. In some embodiments, the stacked plurality of modified amino acids comprises a double-stranded priming region. In some embodiments, the plurality of monomers comprises a plurality of nucleotides, and the monomer is incorporated adjacent to the double-stranded priming region. In some embodiments, (c) is performed using a polymerizing enzyme. In some embodiments, the polymerizing enzyme is a polymerase. In some embodiments, the monomer is a nucleotide or a modified nucleotide, and, during (c), the polymerase incorporates a single nucleotide or modified nucleotide to the polymerizable molecule. In some embodiments, the plurality of nucleic acid molecules comprises an error correction barcode sequence. In some embodiments, the error correction barcode sequence comprises a Hamming code, a Levenshtein code, or a Reed-Solomon code.

In some embodiments, a first end and a second end of the stacked plurality of modified amino acids comprises a blocking region. In some embodiments, the blocking region comprises a hairpin structure, a G-quadruplex, or a protein. In some embodiments, (d) occurs in a cis-to-trans direction. In some embodiments, (d) is performed by applying an electric potential to the nanopore. In some embodiments, (e) comprises reversing the electric potential, thereby translocating the partially polymerized stacked plurality of modified amino acids in a trans-to-cis direction. In some embodiments, (b)-(f) are performed in a single reaction mixture.

In some embodiments, the method further comprises generating the stacked plurality of modified amino acids. In some embodiments, the method further comprises re-sequencing the stacked plurality of modified amino acids, wherein the re-sequencing comprises removing monomers from the partially polymerized stacked plurality of modified amino acids, thereby re-generating the stacked plurality of modified amino acids of (a), and repeating (b)-(e). In some embodiments, the re-sequencing further comprises measuring an additional signal of or adjacent to the nanopore, thereby generating an additional measured signal. In some embodiments, the method further comprises using the measured signal and the additional measured signal to identify an amino acid type of the stacked plurality of modified amino acids, wherein an identification accuracy of the amino acid type is higher using the measured signal and the additional measured signal as compared to an identification accuracy using only the measured signal or only the additional measured signal. In some embodiments, the method further comprises performing the re-sequencing at least 5 times, wherein a consensus accuracy of the re-sequencing for a given amino acid type exceeds 80% for at least 10 distinct amino acid types.

In some embodiments, the nanopore is an MspA nanopore or variant thereof.

In some embodiments, the method further comprises performing differential labeling of the stacked plurality of modified amino acids to confirm the identity of at least one modified amino acid of the plurality of modified amino acids.

In some aspects of the present disclosure, provided herein is a method for processing a modified amino acid, comprising: (a) in an incorporate position, contacting the modified amino acid with a plurality of monomers and a polymerizing enzyme, wherein the modified amino acid comprises a polymerizable molecule and wherein, in the incorporate position, the polymerizing enzyme incorporates n monomers into or adjacent to the modified amino acid, thereby generating a partially polymerized modified amino acid, wherein n is an integer between 0 and 10; (b) translocating the partially polymerized modified amino acid or a portion thereof through a nanopore to a read position; and (c) in the read position, measuring a signal from or adjacent to the nanopore.

In some embodiments, the method further comprises repeating (a) and (b). In some embodiments, given a number of repeated cycles of (a) and (b), the polymerizing enzyme incorporates n monomers according to a Poisson distribution. In some embodiments, an average value of n across the repeated cycles is less than 1. In some embodiments, the polymerizable molecule comprises a nucleic acid molecule. In some embodiments, the nucleic acid molecule comprises an error correction barcode sequence. In some embodiments, the error correction barcode sequence comprises a Hamming code, a Levenshtein code, or a Reed-Solomon code. In some embodiments, the method further comprises, prior to (a), providing a plurality of modified amino acids including the modified amino acid, and performing differential labeling of the plurality of modified amino acids to confirm the identity of at least one modified amino acid of the plurality of modified amino acids.

In some aspects of the present disclosure, provided herein is a method for identifying an amino acid type comprised by a modified amino acid, comprising: (a) translocating, in a first direction, the modified amino acid through a nanopore, wherein the modified amino acid comprises a polymerizable molecule; (b) during (a), measuring a signal from or adjacent to the nanopore, thereby generating a measured signal; (c) translocating the modified amino acid in a second direction; (d) repeating (a)-(c), thereby generating a plurality of measured signals, wherein the plurality of signals, at a given position, have a coefficient of variation of less than about 15%; and (e) using the plurality of measured signals to identify the amino acid type.

In some embodiments, the coefficient of variation is less than about 10%. In some embodiments, the coefficient of variation is less than about 5%. In some embodiments, the polymerizable molecule comprises a nucleic acid molecule. In some embodiments, the nucleic acid molecule comprises an error correction barcode sequence. In some embodiments, the error correction barcode sequence comprises a Hamming code, a Levenshtein code, or a Reed-Solomon code. In some embodiments, the method further comprises, prior to (a), providing a plurality of modified amino acids including the modified amino acid, and performing differential labeling of the plurality of modified amino acids to confirm the identity of at least one modified amino acid of the plurality of modified amino acids.

In some aspects of the present disclosure, provided herein is a method for identifying one or more amino acid types comprised by a plurality of identical modified amino acids (e.g., discrete or individual modified amino acids that comprise the same amino acid type and polymerizable molecules), comprising: (a) translocating the plurality of identical modified amino acids through one or more nanopores; (b) during (a), measuring a signal from or adjacent to the one or more nanopores, thereby generating a plurality of measured signals corresponding to each modified amino acid of the plurality of identical modified amino acids, wherein the plurality of measured signals, at a given position, have a coefficient of variation of less than about 15%; and (c) using the plurality of measured signals to identify the one or more amino acid types.

In some embodiments, the coefficient of variation is less than about 10%. In some embodiments, the coefficient of variation is less than about 5%. In some embodiments, each modified amino acid of the plurality of identical modified amino acids comprises a nucleic acid molecule. In some embodiments, the nucleic acid molecule comprises an error correction barcode sequence. In some embodiments, the error correction barcode sequence comprises a Hamming code, a Levenshtein code, or a Reed-Solomon code. In some embodiments, the method further comprises performing differential labeling of the plurality of identical modified amino acids to confirm the identity of the one or more amino acid types comprised by the plurality of identical modified amino acids.

In some aspects of the present disclosure, provided herein is a method for improving accuracy of identification of a modified amino acid, comprising: (a) providing the modified amino acid and a plurality of monomers, wherein the modified amino acid comprises a polymerizable molecule; (b) translocating the modified amino acid or a portion thereof through a nanopore; (c) measuring a plurality of signals under a plurality of applied voltages at or adjacent to the nanopore, thereby generating a plurality of measured signals; and (d) using the plurality of measured signals to identify an amino acid type of the modified amino acid, wherein an accuracy of identification of the amino acid type is higher using the plurality of measured signals from the plurality of applied voltages as compared to an accuracy of identification using measured signals from a single applied voltage.

In some embodiments, the plurality of applied voltages is a time-varying voltage. In some embodiments, (c) occurs while the modified amino acid is positioned in a constriction region of the nanopore. In some embodiments, the plurality of applied voltages is in a range of about 20 mV to about 120 mV. In some embodiments, the polymerizable molecule comprises a nucleic acid molecule. In some embodiments, the nucleic acid molecule comprises an error correction barcode sequence. In some embodiments, the error correction barcode sequence comprises a Hamming code, a Levenshtein code, or a Reed-Solomon code. In some embodiments, the method further comprises, prior to (a), providing a plurality of modified amino acids including the modified amino acid, and performing differential labeling of the plurality of modified amino acids to confirm the identity of at least one modified amino acid of the plurality of modified amino acids.

In some aspects of the present disclosure, provided herein is a method for processing a modified amino acid, comprising: (a) providing the modified amino acid and a plurality of monomers, wherein the modified amino acid comprises a nucleic acid molecule; (b) coupling a plurality of primers to the nucleic acid molecule, thereby generating a plurality of double-stranded regions along the nucleic acid molecule; and (c) translocating the partially polymerized modified amino acid or a portion thereof through a nanopore, wherein the translocating is performed in absence of a motor protein.

In some embodiments, the double-stranded region is configured to position a portion of the modified amino acid comprising an amino acid in a constriction region of the nanopore. In some embodiments, the nucleic acid molecule comprises an error correction barcode sequence. In some embodiments, the error correction barcode sequence comprises a Hamming code, a Levenshtein code, or a Reed-Solomon code. In some embodiments, the method further comprises, prior to (a), providing a plurality of modified amino acids including the modified amino acid, and performing differential labeling of the plurality of modified amino acids to confirm the identity of at least one modified amino acid of the plurality of modified amino acids.

In some aspects of the present disclosure, provided herein are kits comprising one or more components of the systems, compositions, and methods provided herein. One or more kits may include any useful combination of reagents, analytes, buffer compositions, temperatures, and system components. In some embodiments, the kit comprises reagents for performing intramolecular expansion of a peptide. In some embodiments, the kit comprises reagents for sequencing a modified amino acid or stacked plurality of modified amino acids. In some embodiments, the reagents for sequencing a modified amino acid or stacked plurality of modified amino acids comprises a reaction mixture comprising a plurality of monomers (e.g., nucleotides), a polymerizing enzyme, primers, reagent buffers, or a combination thereof. In some embodiments, the kit may further comprise a flow cell comprising a nanopore, a membrane, a sensor, a power source, or any component of a nanopore sequencer. In some aspects of the present disclosure, provided herein is a system configured to process a modified amino acid, comprising: the modified amino acid, wherein the modified amino acid comprises a polymerizable molecule, wherein the modified amino acid is derived from a peptide analyte; a plurality of monomers; a polymerizing enzyme configured to incorporate a monomer of the plurality of monomers into or adjacent to the polymerizable molecule; and a nanopore.

In some embodiments, the system further comprises a voltage source configured to apply a plurality of voltages across the nanopore. In some embodiments, the system further comprises a measurement circuit configured to measure an ionic current through the nanopore. In some embodiments, a sampling rate of the ionic current through the nanopore is at least 1 kHz. In some embodiments, the system further comprises a processor configured to determine an amino acid type comprised by the modified amino acid. In some embodiments, the polymerizable molecule comprises a nucleic acid molecule. In some embodiments, the nucleic acid molecule comprises an error correction barcode sequence. In some embodiments, the error correction barcode sequence comprises a Hamming code, a Levenshtein code, or a Reed-Solomon code.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A schematically shows an example workflow for processing polymeric analytes (e.g., peptides) described herein.

FIG. 1B schematically shows an example workflow for processing polymeric analytes in solution and on a substrate.

FIG. 1C schematically shows an example of a polymerization-based nanopore sequencing of a stacked plurality of modified amino acids.

FIG. 1D schematically shows an example of a primer-based translocation control approach for nanopore sequencing of a modified amino acid in the absence of a motor protein.

FIG. 2A schematically shows another example workflow for processing polymeric analytes molecules (e.g., peptides) described herein.

FIG. 2B schematically shows another example workflow for processing polymeric analytes on a substrate.

FIG. 2C schematically shows another example workflow for processing polymeric analytes and detection.

FIG. 3 schematically shows a computer system that is programmed or otherwise configured to implement methods provided herein.

FIG. 4A shows example data from a polymerization-based nanopore sequencing readout of a modified amino acid.

FIG. 4B shows example data from a polymerization-based nanopore sequencing readout of five different modified amino acids at different voltages.

FIG. 4C shows example data from a polymerization-based nanopore sequencing readout of five different modified amino acids overlayed together.

FIG. 4D shows example data from a polymerization-based nanopore sequencing readout of a stacked plurality of modified amino acids comprising two modified amino acids.

### DETAILED DESCRIPTION

Provided herein are methods, systems, compositions, and kits for characterizing polymeric analytes, such as proteins. The methods, systems, compositions, and kits of the present disclosure provide for the analysis of individual or clusters of monomers of a polymeric analyte, e.g., amino acids of a protein, thereby providing information on the identity or sequence of monomers (e.g., amino acids in a protein, also referred to herein as “protein sequencing”). Alternatively, or in addition to, the present disclosure may provide for methods of identifying a polymeric analyte without determining the identity of the individual monomers. In some aspects, a method of the present disclosure comprises providing a polymeric analyte, such as a peptide, and determining the identity of the individual (or clusters of) monomers comprised by the polymeric analyte. The present disclosure also provides for approaches for processing and analyzing polymeric analytes, e.g., peptides, polymers, nucleic acid molecules, etc. in a highly-parallelized and accurate manner. Systems and methods of the present disclosure may comprise cleaving a monomer from the polymeric analyte, coupling (e.g., via local tethering) of the monomer to a capture moiety, and detecting the monomer.

In some aspects, the methods, systems, compositions, and kits provided herein entail coupling polymerizable molecules to monomers (e.g., amino acids) of a polymeric analyte, thereby generating modified monomers (e.g., modified amino acids), and detecting the modified monomers (e.g., modified amino acids) or derivative thereof. In some embodiments, the methods provided herein entail repeating one or more operations on a single or plurality of peptides to generate a plurality of modified monomers, which may be discrete or which may be tethered together (e.g., in a stacked plurality of modified monomers), and analyzing or detecting the plurality of modified monomers. In some embodiments, the detecting is performed using nanoscale objects (e.g., nanopores or nanogaps), imaging (e.g., fluorescence imaging), spectroscopy or spectrometry (e.g., mass spectrometry), nucleic acid sequencing, or a combination thereof.

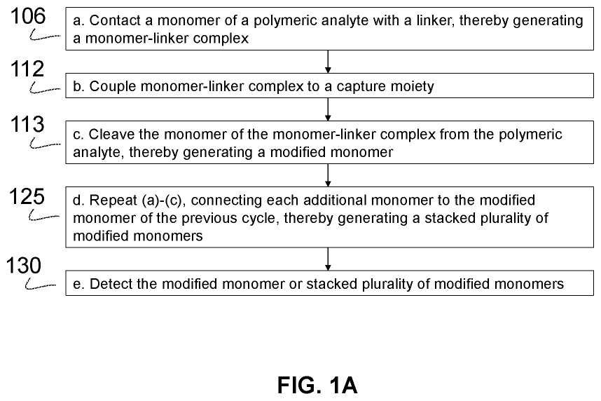

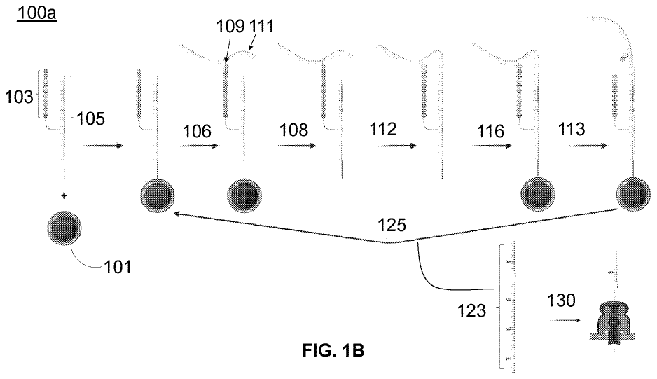

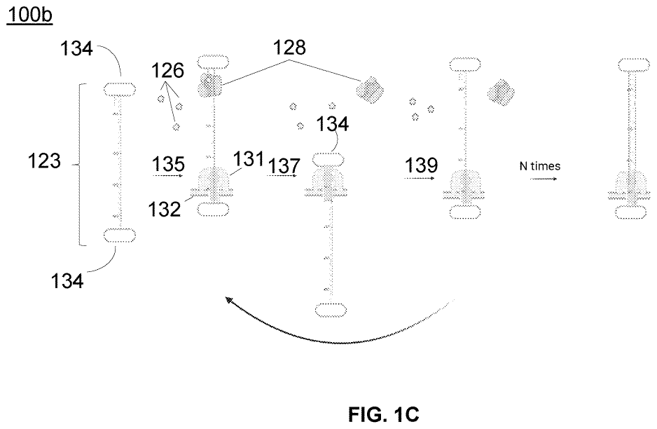

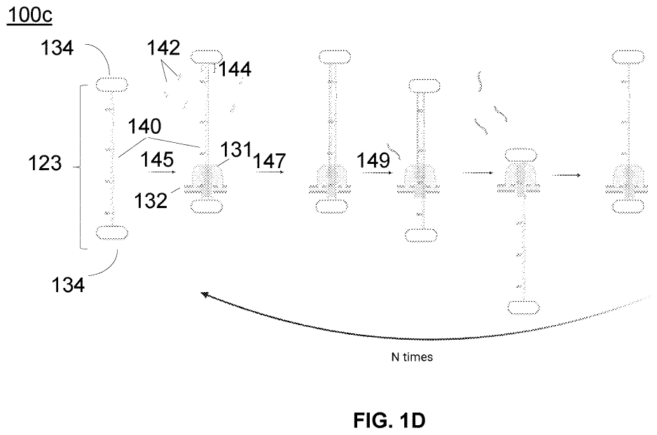

In some aspects, the methods provided herein comprise performing an intramolecular expansion of a polymeric analyte, e.g., a peptide, to generate a modified monomer (e.g., modified amino acid), e.g., as shown in FIGS. 1A-1B. Such an intramolecular expansion process may comprise providing a linker, coupling the linker to a monomer of the polymeric analyte to generate a monomer-linker complex, coupling the linker or the monomer-linker complex to a capture moiety, and cleaving the monomer from the polymeric analyte, thereby yielding a modified monomer. In some instances, the method may further comprise repeating the intramolecular expansion or one or more operations of the intramolecular expansion on the next monomer of the polymeric analyte to generate another modified monomer. In some instances, the monomer-linker complex or the modified monomer resulting from a round or cycle of intramolecular expansion may be coupled to that of the previous round or cycle of intramolecular expansion to generate a stacked plurality of modified monomers, e.g., linked by a polymerizable molecule backbone. The modified monomer or stacked plurality of modified monomers may be detected using a suitable approach to output the identity of the modified monomer (e.g., an amino acid type of a modified amino acid). The detection may be performed using imaging, nanopore sequencing, spectrometry or spectroscopy, or other suitable detection technique.

In some examples, the methods provided herein may allow for sequencing a peptide comprising a plurality of amino acids. For example, a method may comprise (a) providing a plurality of modified amino acids generated from at least a subset of the plurality of amino acids, and (b) determining an amino acid identity (e.g., an amino acid type) of each modified amino acid of the plurality of modified amino acids. The methods provided herein may advantageously provide for highly-accurate identification and sequencing of individual amino acids from a peptide; for instance, sequencing of the plurality of modified amino acids may have an average read accuracy that is greater than 80% for at least 2 different modified amino acid types. The plurality of modified amino acids may be derived from two or more contiguous or non-contiguous amino acids of a peptide. In some embodiments, the modified amino acids herein comprise polymerizable molecules.

Modified amino acids: In some aspects, the modified monomer is a modified amino acid. The modified amino acid or derivative thereof may originate from or be part of a protein or peptide; for example, the modified amino acid may comprise or be derived from an amino acid located at a terminus (N-terminus or C-terminus) of a peptide, or the modified amino acid may comprise or be derived from an amino acid located within the peptide. The modified amino acid may comprise a proteinogenic amino acid or derivative thereof with any number of modifications. Examples of modifications include, in non-limiting examples, chemical modifications (e.g., protecting groups), biological modifications (e.g., post-translational modifications, modifications introduced by enzymatic treatment or digestion), physical modifications (e.g., mutations introduced by irradiation, heat, etc.), and the like. In some instances, the modified amino acid or derivative thereof comprises or is coupled to a binding agent, such as an antibody, antibody fragment, nanobody, aptamer, peptide, a small molecule, an inorganic compound, a polymer, or any variations or combinations thereof. The modified amino acid may comprise a covalent or noncovalent modification. In some instances, the modified amino acid comprises a non-naturally occurring chemical modification. For example, the modified amino acid may comprise a protecting group, such as, in non-limiting examples, a methyl, formyl, ethyl, acetyl, t-butyl, anisyl, benzyl, trifluoroacetyl, N-hydroxysuccinimide, t-butyloxycarbonyl, benzoyl, 4-methyl benzyl, thioanizyl, thiocresyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl, triphenylmethyl, or 2,2,5,7,8-pentamethyl-chroman-6-sulphonyl group. In some instances, the modified amino acid comprises a proteinogenic amino acid or derivative thereof that is coupled to a polymerizable molecule. In some instances, the modified amino acid comprises a proteinogenic amino acid or derivative thereof, a linker, and a polymerizable molecule.

The modified amino acid may comprise any useful modification. Modifications may be naturally-occurring (e.g., post translational modifications) or non-naturally occurring, such as by labeling or tagging, e.g., with an amino acid- or amine-reactive agent or linker comprising the amino acid- or amine-reactive agent. Examples of amino acid- or amine-reactive agents include isothiocyanate (e.g., PITC, NITC), 1-fluoro-2,-4-dinitrobenzene (DNFB), dansyl chloride, 4-sulfonyl-2-nitrobfluorobenzene (SNFB), an acetylating agent, an acylating agent, an alkylating agent, a guanidination agent, a thioacetylation agent, a thioacylation agent, a thiobenzoylation agent, or a derivative or combination thereof. Alternatively, or in addition to, the one or more modified amino acids may comprise an adduct (e.g., a polymer such as PEG, a polymerizable molecule such as a nucleic acid molecule, a nanoparticle or nanotube, a peptide or protein), a lipid, a carbohydrate, a metabolite, a fluorophore, a hapten, a quencher, a tag (e.g., a fluorescent tag, a magnetic tag, a radioactive tag), a barcode, or other moiety. In some instances, a modified amino acid may comprise a modification that facilitates recruitment of an enzyme (or ribozyme or DNAzyme) to recognize or cleave a terminal amino acid, e.g., a NTAA or CTAA of a peptide. For example, a terminal amino acid of a peptide may be modified with a saccharide in order to recruit a lectin or lectin-bound protease. In another example, one or more modified amino acids may comprise or be coupled to a nucleic acid molecule having a first sequence that is complementary to a second sequence comprised by an oligo-bound protease. Hybridization of the first sequence to the second sequence may facilitate local recruitment of the protease to the amino acid to be cleaved. In yet another example, a peptide may be modified with phenylisothiocyanate (PITC), which may allow for recruitment and cleavage of the modified amino acid by an Edmanase. In other examples, a peptide may be modified with functional moiety that is recognized by a specific cleaving enzyme; recognition and binding of the cleaving enzyme to the functional moiety may result in cleavage of the modified amino acid. In some examples, modifications to amino acids may include epitope tags, which can facilitate binding of a binding agent to the modified amino acid. Examples of such epitope tags include fluorophores, nucleic acid molecules, peptides, haptens, polymers, chemical moieties, or other adduct molecule.

The methods described herein may further comprise generating the modified amino acid from a peptide, such as a sample peptide or peptide analyte. In some instances, the modified amino acid comprises a proteinogenic amino acid or derivative thereof, a linker, and a polymerizable molecule. In one example, an amino acid of a peptide may be contacted with a linker that comprises (i) first reactive moiety capable of reacting with the amino acid and (ii) a second reactive moiety. Prior to, during, or subsequent to the reaction of the first reactive moiety with the amino acid, a polymerizable molecule comprising a third reactive moiety that is capable of reacting with the second reactive moiety may be provided. The second and third reactive moieties may comprise click chemistry moieties that can react with one another (e.g., azide and DBCO, azide and BCN, alkyne and DBCO, TCO and tetrazine, etc.). The reaction of the amino acid with the linker and the linker to the polymerizable molecule may thus yield a modified amino acid comprising the amino acid, the linker, and the polymerizable molecule. Alternatively, or in addition to, the polymerizable molecule may comprise the amino acid-reactive moiety (optionally via a linker) and may react directly with the amino acid. In some instances, cleavage of the amino acid from the peptide may be performed, and the modified amino acid may comprise the cleaved product comprising the cleaved, and optionally derivatized, amino acid, the linker (if present), and the polymerizable molecule. In some instances, the amino acid is a terminal amino acid.

A modified amino acid may comprise a single amino acid or a plurality of amino acids (e.g., dipeptide, tripeptide, etc.). For example, the modified amino acid may comprise 1, 2, 3, 4, 5, 6, 7, 8, 9, 10 or greater amino acids. In instances where the modified amino acid comprises more than one amino acid, the plurality of amino acids comprised by the modified amino acid may be of the same amino acid type (e.g., leu-leu, val-val, ile-ile) or different amino acid types (e.g., val-pro, arg-his, gly-leu). The plurality of amino acids comprised by the modified amino acid may comprise any number of modifications, e.g., as described elsewhere herein.

Detection or Identification: In some embodiments, the modified monomers (e.g., modified amino acids) may be detected or identified, e.g., determining an amino acid type of one or more of the modified amino acids. Detection may be performed using any useful technique, such as imaging, using a nanopore sensor or sequencer, mass spectrometry, or other detection technique.

Nanopore sensing/sequencing: In some embodiments, the modified monomers (e.g., a modified amino acid or derivative thereof) may be subjected to nanopore sensing or sequencing to characterize and/or determine the identity (e.g., an amino acid type) of the modified amino acid and optionally, of the polymerizable molecule. The nanopore sensing or sequencing may be performed using a single nanopore, a one-dimensional nanopore array, a two-dimensional nanopore array, or a three-dimensional nanopore array. The nanopore sensing or sequencing may be performed using any nanopore system, e.g., Oxford Nanopore Technologies, Illumina, Genia Technologies (also Stratos Genomics and Roche Axelios), Qitan, Puyi Biotech, BGI/MGI CycloneSeq, NobleGen, Electronic Biosciences, IMEC, Northern Nanopore Instruments, Norcada, or Quantum Biosystem. The nanopore system may have a Faradaic or non-Faradaic sensing mechanism or operate on DC or AC voltage. The nanopore sequencer may apply multiple voltages at each position being sequenced to collect varying voltage measurements of the modified monomer. The nanopore sequencer may be able to perform single molecule re-reading of the modified monomer to measure the signal associated with the monomer more than once over a series of translocation events. Nanopore sequencing may be performed to determine the identity of different components of the modified amino acids; for example, a modified amino acid comprising a derivatized amino acid (e.g., a thiocyanate-conjugated amino acid or a thiocarbamyl, thiazolinone, or thiohydantoin derivative) coupled to a polymerizable molecule (e.g., a nucleic acid molecule) may be subjected to nanopore sequencing, which may output the identity of which amino acid type (e.g., which of the 20 proteinogenic amino acids or post-translationally-modified amino acids) the modified amino acid comprises or is derived from or a subset of types (e.g., based on a physicochemical characteristic such as a hydrophobic residue, a charged residue, etc.). Optionally, the identity of individual monomers of the polymerizable molecule (e.g., the nucleic acid sequence of the nucleic acid molecule) may also be identified using nanopore sequencing. For example, the modified amino acids or derivatives thereof may be translocated through or adjacent to a nanopore. As the modified amino acids or derivatives thereof translocate through or adjacent to the nanopore, one or more signals (e.g., current signal, current blockage, impedance, inductance, etc.) generated from both the polymerizable molecule and the amino acid may be generated and collected. Such one or more signals may then be deconvolved using computational approaches to determine the identity of the polymerizable molecules (e.g., nucleic acid sequences) and the amino acid types of the modified amino acids or derivatives thereof. Beneficially, the use of nanopore or nanogap sequencing for generation of multiplexed data may obviate the need for multiple analysis techniques or instruments. In instances where the polymerizable molecule comprises a nucleic acid molecule that encodes additional information (e.g., comprises barcode sequences, UMIs, cycle information, a null event, spatial information etc.), the sequencing of both the derivatized amino acid and the nucleic acid molecules may generate multiplexed information.

The one or more signals output from the nanopore may be measured and used to determine an identity (e.g., the amino acid type) of a modified amino acid. Any useful measurement can be made, e.g., a conductance, current, current blockage, current density, current change, voltage, impedance, resistance, inductance, capacitance, frequency, phase, power, electric field, magnetic field, fluorescence, plasmon-coupled fluorescence, or other parameter or change in the parameter within or adjacent to the nanopore. The measurement may be a function of time. In some instances, the current signal may be an ionic current signal, a cross-pore or transverse-to-pore drain current, or a source current. The current may be multiple ionic currents measured at different voltages on the same molecule. The current may be from the same single molecule but from different reads (re-reading). The measurement may be made from a FET sensing mechanism or voltage sensing mechanism, or from a non-Faradaic or Faradaic current measurement. The measurement may be made using an impedance measurement under an alternating current, e.g., as described in Kitta et al., ACS Omega 2023, 8, 14684-14693. As a molecule (e.g., modified monomer or modified amino acid) enters the nanopore, nanogap, or nanochannel, a change in the conductance, current, impedance, or other parameter may occur and provide information (e.g., size, charge, aspect ratio, volume, hydrophobicity, chemical structure) on the molecule. Each amino acid or modified amino acid, or a subset of amino acids or modified amino acids, may generate a unique signal that is distinguishable from other amino acids or modified amino acids. As such, the unique signal signatures may be assigned to the amino acids or modified amino acids in order to determine the identity of the amino acids, polymerizable molecules (e.g., a polymer identity, a nucleic acid sequence), or both.

In some aspects, a method of the present disclosure comprises providing a modified amino acid, e.g., as described above, and identifying or detecting the identity or amino acid type comprised by the modified amino acid using a polymerization-based nanopore sequencing approach with re-reading, e.g., as described in U.S. Pat. Pub. No. 2025/0215487, which is incorporated by reference herein in its entirety. In some embodiments, the method comprises providing (i) a modified amino acid comprising a polymerizable molecule (e.g., an oligonucleotide) and (ii) a plurality of monomers (e.g., nucleotides); incorporating a monomer of the plurality of monomers into or adjacent to polymerizable molecule (e.g., using a polymerase), thereby generating a partially polymerized modified amino acid; and translocating (e.g., in a cis-to-trans direction) the partially polymerized modified amino acid or a portion thereof through a nanopore. Prior to, during, or subsequent to the translocating, one or more signals may be measured (e.g., a current signal measured at a first applied voltage). In some embodiments, the partially polymerized modified amino acid may be translocated in an opposite direction (e.g., in a trans-to-cis direction), and the process may be repeated, thereby generating a plurality of signals, which may be altogether used to identify an amino acid type comprised by the modified amino acid.

In some embodiments, the modified amino acid (or a partially or fully polymerized modified amino acid, also referred to herein for simplicity as “the modified amino acid”) is translocated between alternating incorporate and read positions (e.g., by reversing the voltage applied across the nanopore). In an incorporate position, a first end of the polymerizable molecule may be accessible to a polymerizing enzyme, e.g., a polymerase, and free monomers (e.g., nucleotides) in the reaction mixture, such that the polymerizing enzyme may bind to the modified amino acid or stacked plurality of modified amino acids, and the polymerizable molecule of the modified amino acid or stacked plurality of modified amino acids may be extended by incorporation of a monomer (e.g., a nucleotide).

As described herein, in some embodiments, a “partially polymerized modified amino acid” refers to a modified amino acid that has had one or more monomers incorporated into or adjacent to the modified amino acid. For instance, the modified amino acid may comprise the cleaved amino acid (e.g., cleaved from a peptide analyte) and further comprise a linker, and a partially single-stranded nucleic acid (polymerizable) molecule. In the incorporate position, the incorporation of one or more nucleotides by the polymerizing enzyme may generate the partially polymerized modified amino acid, e.g., a modified amino acid comprising one or more nucleotides incorporated therein.

The rate of polymerizable molecule extension (incorporation) can depend on the concentrations of polymerase and nucleotides in the reaction mixture, temperature, and other conditions. In some embodiments, a low rate of extension may be used (e.g., by using low concentrations of polymerase and/or nucleotides) such that within a predetermined amount of time, it is highly probable that only one monomer, or no monomer, has been successfully incorporated. In some instances, the rate of extension may follow a Poisson distribution, where the average incorporation rate is less than 1. Alternatively, a higher rate of extension may be favorable if required to increase the speed of sequencing (e.g., allowing for skipping of regions).

In some embodiments, the polymerizing enzyme is a polymerase. The polymerase may be a DNA polymerase, an RNA polymerase, or a reverse transcriptase. Non-limiting examples of DNA polymerases suitable for use in the methods described herein include DNA polymerase I (Pol I), DNA polymerase II (Pol II), DNA polymerase III (Pol III), DNA polymerase IV (Pol IV), Klenow fragment, T4 DNA polymerase, T7 DNA polymerase, phi29 DNA polymerase, Bst DNA polymerase, Bsu DNA polymerase, 9 degrees North (9N) DNA polymerase, Taq DNA polymerase, Vent DNA polymerase, Deep Vent DNA polymerase, Sequenase, Therminator DNA polymerase, or engineered variants thereof. In some embodiments, the polymerase is a strand-displacing polymerase (e.g., phi29, Bst, or 9N polymerase). In some embodiments, the polymerase is selected based on its processivity, fidelity, incorporation rate, or compatibility with modified nucleotides. In some embodiments, the polymerase is a processive polymerase. In some embodiments, the polymerase is a distributive polymerase. The choice of polymerase may affect the rate of monomer incorporation and therefore the speed and resolution of nanopore sequencing. In some instances, a distributive polymerase may be preferred for polymerization-based nanopore sequencing, as it may incorporate fewer monomers per binding event, allowing for more controlled and stepwise extension of the polymerizable molecule. In some instances, the concentration of the polymerase in the reaction mixture may be about 0.01 nM to about 1000 nM, about 0.1 nM to about 100 nM, about 1 nM to about 50 nM, or about 5 nM to about 20 nM. The reaction mixture may further comprise divalent cations (e.g., magnesium ions (Mg2+) or manganese ions (Mn2+)) at concentrations suitable for polymerase activity.

Any useful number of monomers may be incorporated into the modified amino acid by the polymerizing enzyme while the modified amino acid is in the incorporate position. For example, in a given duration of time (e.g., on the order of 1-1000 milliseconds), about 0, about 1, about 2, about 3, about 4, about 5, about 6, about 7, about 8, about 9, about 10 or more monomers may be incorporated into the modified amino acid. The number of monomers incorporated may be stochastic or may follow a Poissonian distribution. In some embodiments, for a given duration of time, about 0 or about 1 monomer is incorporated into the modified amino acid. In some embodiments, for M incorporation-read cycles, where M is an integer greater than 1, the average number of monomer incorporations is between 0 and 1.

In some embodiments, in the incorporate position of an incorporate-read cycle, the polymerizing enzyme incorporates n monomers into or adjacent to the modified amino acid, wherein n is an integer between 0 and 10, between 0 and 5, between 0 and 3, or between 0 and 2. In some embodiments, n is 0 or 1 for a majority of incorporate-read cycles. The number of monomers incorporated per cycle may follow a Poisson distribution, wherein the mean (lambda) of the Poisson distribution is less than about 2, less than about 1.5, less than about 1, less than about 0.8, less than about 0.5, less than about 0.3, or less than about 0.1. As such, the probability of incorporating exactly 0 monomers and exactly 1 monomer may together constitute a majority of outcomes. For instance, with a Poisson mean of lambda=0.5, the probability of incorporating exactly 0 monomers is approximately 61%, and the probability of incorporating exactly 1 monomer is approximately 30%, such that about 91% of cycles result in 0 or 1 monomer incorporations. The Poisson statistics of monomer incorporation may be tuned by adjusting the concentrations of the polymerizing enzyme and/or the plurality of monomers in the reaction mixture, the duration of the incorporate phase, the temperature, the reagent components, or other reaction conditions. In some embodiments, the incorporate phase duration may be any useful duration, e.g., on the millisecond, second, or minutes scale.

After a predetermined amount of time, the modified amino acid may be translocated (e.g., by applying a force such as an electric potential to translocate the modified amino acid in a cis-to-trans direction) to a read position. In the read position, a portion of the modified amino acid (e.g., at least a portion of the polymerizable molecule) may be lodged within the aperture of the nanopore, and a signal (e.g., ionic current) measurement is made. In the read position, the polymerizing enzyme may be dislodged from the modified amino acid or stacked plurality of modified amino acids so that the polymerizable molecule cannot be extended with a monomer during the read position.

Subsequently, the modified amino acid may be translocated in a second direction (e.g., a trans-to-cis direction) such that the polymerizable molecule is dislodged from the aperture of the nanopore, and the modified amino acid is returned to the incorporate position, so that a polymerase from the reaction mixture may bind and the polymerizable molecule may be extended again. The modified amino acid may once again be translocated to the read position. The iteration or repeating of incorporating and reading (“incorporate-read cycles”) allows for repeated measurements of the modified amino acid. Beneficially, the polymerization-based nanopore sequencing approach allows for re-reading of the same modified amino acid, thereby allowing for greater data acquisition and more accurate determination of the amino acid type comprised by the modified amino acid. Moreover, the disclosed methods are compatible with many incorporate-read cycles, e.g., of up to about 1,000 monomer incorporation cycles, or up to about 2,000 cycles, or up to about 5,000 cycles or more. Beneficially, using a polymerization-based nanopore sequencing approach also allows for controllable and reproducible measurements, improving the temporal precision of measurements, and avoiding or accounting for translocation events that are too fast to be detected. Further, the use of a polymerase-based approach, rather than a helicase ratcheting mechanism, may decrease errors associated with helicase skipping or stalling.

Measurement of a signal (e.g., a current or ionic signal) of or adjacent to the nanopore may be performed discretely or continuously. In some embodiments, the measuring of the signal occurs during translocating of the modified amino acid or portion thereof. In some embodiments, the measuring of the signal occurs while the modified amino acid or portion thereof is arrested at a constriction region of the nanopore. Alternatively, or in addition to, the measurement of the signal may be continuous and may occur while the modified amino acid is in the read position, the incorporate position, or any intermediary position. In some embodiments, the measured signal is used to identify an amino acid type of the modified amino acid. In some embodiments, the measured signal is a current signal under an applied voltage. In some embodiments, the method further comprises measuring a plurality of signals of or adjacent to the nanopore under a plurality of applied voltages, and using the plurality of signals to identify an amino acid type of the modified amino acid.

In some embodiments, a characteristic or statistic of one or more measured signals can be used to identify the amino acid type of the modified amino acid. For example, a mean current signal of a plurality of current signal traces arising from the same modified amino acid may be used to identify or distinguish an amino acid type. Similarly, a standard deviation of a plurality of current signal traces arising from the same modified amino acid may be used. Any useful metric or characteristic of the one or more measured signals (e.g., current, conductance, impedance) may be used, e.g., a raw value, a mean value (μ_{signal}), a median value, a range of values (e.g., max signal-min signal), a standard deviation or noise of the signal (σ_{signal}), a coefficient of variation (σ_{signal}/μ_{signal}×100), a signal-to-noise ratio (μ_{signal}/σ_{signal}), peak width, peak signal, full width half max, peak position, separation resolution, area-under-the-curve (AUC), or other value or combination of values. In some instances, a ratio of the current and the voltage, e.g., a conductance (current (I)/voltage (V)) or a change or rate of change in conductance (e.g., slope of the current/voltage or dI/dV) can be used to identify the amino acid type. In some instances, more than one metric may be used to identify the amino acid type comprised by a modified amino acid.

In some embodiments, a coefficient of variation (CV) of the measured signals may be used to identify or distinguish amino acid types. The coefficient of variation may be calculated as the ratio of the standard deviation of the signal to the mean of the signal, expressed as a percentage (σ_{signal}/μ_{signal}×100). In some embodiments, the CV of a plurality of measured signals arising from the same modified amino acid at a given position is less than about 20%, less than about 15%, less than about 10%, less than about 8%, less than about 6%, less than about 5%, less than about 4%, less than about 3%, less than about 2%, or less than about 1%. In some instances, the CV varies as a function of the applied voltage; for instance, at lower applied voltages (e.g., about 40 mV), the CV may be higher due to smaller mean currents while noise remains relatively constant, whereas at moderate applied voltages (e.g., about 80 mV), the baseline CV may be lower but amino acid-specific dips may generate distinguishable CV perturbations. In some embodiments, each amino acid type has a distinguishable CV fingerprint or profile when considering a plurality of applied voltages, and the distinguishable CV profiles may serve as additional discriminating features for amino acid type identification. For example, amino acids with bulky or charged side chains (e.g., arginine, phenylalanine) may exhibit broader or higher CV perturbations than amino acids with smaller side chains (e.g., glycine, alanine).

Beneficially, the methods provided herein provide for highly accurate and reproducible measurements of modified amino acids. For instance, a coefficient of variation of a plurality of measured signals at a given position (e.g., far from the amino acid site, at the amino acid site, adjacent to the amino acid site) from the same modified amino acid may be less than about 20%, less than about 15%, less than about 10%, less than about 5%, or less than about 1% for a given applied voltage. Low CV values for one or more positions in the same measured modified amino acid or for identical but discrete modified amino acids (e.g., modified amino acids that have the same amino acid type and same nucleotide backbone) indicates a robust and reproducible method for measuring signal and identification accuracy.

In some embodiments, the methods provided herein exhibit high inter-molecule reproducibility, wherein a plurality of identical modified amino acids (e.g., modified amino acids that share the same amino acid type and the same nucleotide backbone sequence) are translocated through one or more nanopores, and the measured signals corresponding to each modified amino acid of the plurality of identical modified amino acids have a coefficient of variation of less than about 15%, less than about 10%, less than about 8%, less than about 5%, less than about 3%, or less than about 1% at a given position for a given applied voltage. Such inter-molecule reproducibility indicates that the nanopore-based signal measurements are robust and consistent across discrete molecular events, which is beneficial for accurate amino acid type identification from measured signals.

In some embodiments, the plurality of identical modified amino acids is translocated through one or more nanopores. For instance, the plurality of identical modified amino acids (e.g., of a mixed population of modified amino acids) may be translocated sequentially through a single nanopore, or may be translocated in parallel through a plurality of nanopores (e.g., a nanopore array). In some embodiments, the one or more nanopores comprises a nanopore array comprising at least 2, at least 10, at least 100, at least 1,000, at least 10,000, least 100,000, at least 500,000, or at least 1,000,000 individually addressable nanopores, each nanopore having an independently controllable voltage source and measurement circuit and which optionally may be provided across a single or a plurality of flowcells. In some embodiments, each nanopore of the nanopore array is an MspA nanopore or variant thereof. Parallel measurement of a plurality of identical modified amino acids through a nanopore array may increase throughput and provide statistical sampling of signal characteristics across multiple independent measurement events.

The methods and systems provided herein may obtain any useful throughput of modified amino acid measurements or calls per unit time, e.g., at least about 1 measurement (pertaining to 1 nucleotide or 1 modified amino acid) per 10 minutes, per 1 minute, per 100 milliseconds (ms), per 10 ms, or per 1 ms.

In some embodiments, the plurality of measured signals corresponding to each modified amino acid of the plurality of identical modified amino acids are used to identify one or more amino acid types comprised by the plurality of identical modified amino acids. For instance, a mean, median, or consensus of the plurality of measured signals may be computed across the plurality of identical modified amino acids, and the resulting signal metric may be used to identify or distinguish amino acid types with improved accuracy relative to a signal from a single modified amino acid. In some embodiments, the coefficient of variation across the plurality of measured signals at a given position serves as a quality metric, wherein a low coefficient of variation (e.g., less than about 15%, less than about 10%, or less than about 5%) indicates that the measured signals are suitable for amino acid type identification. In some embodiments, if the coefficient of variation exceeds a threshold, additional measurements may be obtained (e.g., by translocating additional identical modified amino acids) until the coefficient of variation falls below the threshold.

As described elsewhere herein, in some instances, the polymerizable molecule comprises a nucleic acid molecule. In some embodiments, the nucleic acid molecule is at least partially single-stranded. In some embodiments, the nucleic acid molecule comprises a double-stranded priming region. In some embodiments, the nucleic acid molecule is partially single-stranded and a primer may hybridize to a portion of the partially single-stranded region (e.g., at or toward an end of the nucleic acid molecule), thereby forming a double-stranded priming region. In some embodiments, the modified amino acid comprises a partially single-stranded nucleic acid molecule comprising a double-stranded priming region at the first end, and in the incorporate position, a polymerizing enzyme, e.g., polymerase, binds to the double-stranded priming region at the first end and incorporates a single nucleotide to the nucleic acid molecule, thereby extending the double-stranded priming region by one nucleotide. The modified amino acid is then translocated (e.g., in a cis-to-trans direction) to the read position in the nanopore aperture. The double-stranded region arrests translocation of the first end of the modified amino acid through the nanopore, as only single stranded nucleic acid molecules may translocate through the constriction region of the nanopore.

In some instances, a modified amino acid may additionally comprise one or more blocking regions, which may comprise a structural motif that prevents the modified amino acid from exiting the nanopore. The blocking region may be disposed at a first end, a second end, or both ends of the modified amino acid. The blocking region may comprise any useful structural moiety or feature, e.g., a hairpin loop, a protein (e.g., biotin, streptavidin), a bead, a particle, a double stranded nucleic acid moiety, a quadruplex (e.g., G-quadruplex) or other bulky moiety. In some instances, the blocking region comprises a hairpin loop.

In some embodiments, the method further comprises coupling a blocking region to the modified amino acid. For instance, subsequent to generation of the modified amino acid, the modified amino acid may be contacted with a blocking region. In some instances, the blocking region comprises a single-stranded primer, which may couple via hybridization to a complementary sequence comprised by the modified amino acid (e.g., by the polymerizable molecule). In another example, the blocking region (e.g., hairpin moiety) may be ligated to the modified amino acid. In yet another example, the blocking region may be provided in the trans compartment of a nanopore, such that subsequent to the modified amino acid entering the aperture of the nanopore and at least partial translocation of the modified amino acid in the first direction (e.g., cis to trans), the blocking region may hybridize or ligate to the modified amino acid, thereby preventing the modified amino acid from exiting the nanopore when translocating in the second direction (e.g., trans to cis).

In some embodiments, the modified amino acid is comprised by a stacked plurality of modified amino acids. In such instances, the stacked plurality of modified amino acids may comprise a first end and a second end, in which the first end or the second end comprises a blocking region which prevents the stacked plurality of modified amino acids from escaping the nanopore.

Translocation of the modified amino acid may occur by applying a force to the modified amino acid (e.g., a pressure, an electric potential, a gravitational force, a centrifugal force, or other force). In some embodiments, application of an electric potential is used to translocate the modified amino acid in a cis-to-trans direction (e.g., to translocate the modified amino acid to a read position). In some embodiments, the method further comprises reversing the electric potential, thereby translocating the modified amino acid in a trans-to-cis direction (e.g., to translocate the modified amino acid to an incorporate position). In some embodiments, the incorporating and translocating steps are then repeated, enabling iterative, bidirectional ratcheting of the modified amino acid through the nanopore for improved sequencing accuracy.

In some embodiments, the incorporating and translocating, or any operations of one or more incorporate-read cycles, are performed in a single reaction mixture comprising the polymerizing enzyme and the plurality of monomers (e.g., nucleotides). The reaction mixture may comprise any useful composition and may have the appropriate temperature, pH, buffer composition, salts, or any useful reagent to perform the operations described herein.

In some embodiments, the method further comprises generating the modified amino acid, as described elsewhere herein. In some embodiments, generating the modified amino acid comprises: providing a linker and a polymerizable molecule; coupling the linker to (i) an amino acid of a peptide and (ii) the polymerizable molecule to generate an amino acid-linker complex; and cleaving the amino acid from the peptide, thereby generating the modified amino acid comprising the cleaved amino acid (also referred to herein as the “amino acid portion” or “amino acid site” of the modified amino acid), the linker, and the polymerizable molecule. In some embodiments, coupling of the linker to the polymerizable molecule occurs prior to coupling to the amino acid.

In some aspects, a method of the present disclosure comprises identifying a stacked plurality of modified amino acids. As described elsewhere herein, a stacked plurality of modified amino acids may comprise a plurality of modified amino acids; as such, the identifying may comprise identification of an amino acid type of each individual modified amino acid in the stacked plurality of modified amino acids. The plurality of modified amino acids may comprise a plurality of polymerizable molecules, which may be coupled to one another in a linear fashion. The stacked plurality of modified amino acids translocated through a nanopore for sequencing, e.g., using the iterative incorporate-read cycles. In some embodiments, the method comprises: (a) providing the stacked plurality of modified amino acids; (b) contacting the stacked plurality with a plurality of monomers; (c) incorporating a monomer of the plurality of monomers into or adjacent to a polymerizable molecule of the plurality of polymerizable molecules, thereby generating a partially polymerized stacked plurality of modified amino acids; (d) translocating, in a first direction, the partially polymerized stacked plurality or a portion thereof through a nanopore; (e) translocating, in a second direction, the partially polymerized stacked plurality or a portion thereof through the nanopore; and (f) repeating (c)-(e).

Helper Molecules and Primer-Based Translocation Control: In some aspects, the methods provided herein may comprise the use of one or more helper molecules. The modified amino acid or stacked plurality of modified amino acids may be contacted with one or more helper molecules, e.g., as described in International Pat. Pub. No. WO/2025166050, which is incorporated by reference herein. The helper molecules can aid in reducing or halting the translocation and thus improve the accuracy of the readout obtained from nanopore sequencing. In some instances, the one or more helper molecules comprise a nucleic acid molecule (e.g., a DNA oligonucleotide or RNA oligonucleotide) that can hybridize to the polymerizable molecule (e.g., another DNA molecule) of the modified amino acid or stacked plurality of modified amino acids. The hybridization of the helper molecule to the polymerizable molecule may generate a double-stranded region along the polymerizable molecule. In some embodiments, the modified amino acid comprises a nucleic acid molecule (e.g., a single-stranded DNA molecule), and a plurality of primers or short oligonucleotides are coupled (e.g., via hybridization) to the nucleic acid molecule, thereby generating a plurality of double-stranded regions along the nucleic acid molecule. The plurality of double-stranded regions may act as structural impediments that modulate the translocation of the modified amino acid through the nanopore, as the double-stranded regions cannot pass through the constriction region of the nanopore without first being denatured or sheared off. As such, the plurality of double-stranded regions may control the step-wise movement of the modified amino acid through the nanopore, positioning different portions of the modified amino acid (e.g., the amino acid portion) in or adjacent to the constriction region for signal measurement.

In some embodiments, the double-stranded regions are configured to position a portion of the modified amino acid comprising an amino acid in the constriction region of the nanopore. For instance, the primers may be designed to hybridize at specific positions along the nucleic acid molecule such that, when the modified amino acid is translocated through the nanopore, each double-stranded region arrests translocation at a defined position, thereby positioning the amino acid moiety in or near the constriction zone for optimal signal generation. The primers may be of any useful length, e.g., about 5 to about 50 nucleotides, about 8 to about 30 nucleotides, or about 10 to about 20 nucleotides. The melting temperature (Tm) of the primers may be designed to be in a range that allows for controlled, sequential dissociation under denaturing conditions (e.g., an applied electric force, toehold displacement, chemical or heat denaturation). In some embodiments, the primers may comprise modified nucleotides (e.g., locked nucleic acids, 2-O-methyl nucleotides, or phosphorothioate-modified nucleotides) to increase the thermal stability of the double-stranded regions.

Beneficially, the use of a plurality of primers to generate double-stranded regions along the nucleic acid molecule may obviate the need for a motor protein (e.g., a helicase or other molecular motor) to control translocation velocity. In conventional nanopore sequencing approaches, motor proteins such as helicases are used to ratchet a nucleic acid strand through the nanopore at a controlled rate. However, motor proteins may introduce errors, such as skipping or backstep events, and may have limited processivity. By instead using primer-generated double-stranded regions for translocation control, the methods described herein may achieve reproducible and accurate positioning of the modified amino acid in the nanopore constriction region without the variability associated with motor protein activity. In some embodiments, the translocating of the modified amino acid through the nanopore is performed in the absence of a motor protein. In some embodiments, the translocating is driven solely by the applied electric potential, and the step-wise positioning is controlled by the sequential dissociation of the double-stranded regions as the modified amino acid is pulled through the nanopore under the applied force. Such a primer-based translocation control mechanism may also simplify the reaction mixture and reduce the cost and complexity of the sequencing workflow.

Additional example methods and systems of nanopore sequencing of polymeric analytes are described in U.S. Pat. Nos. 12,259,393 and 12,259,394 and International Patent Pub. No. WO2025/166050, each of which is incorporated by reference herein in its entirety.

FIGS. 1A-1B schematically show example workflows for generating a modified monomer from a polymeric analyte (e.g., a modified amino acid from a peptide), which may occur entirely or partially on a substrate or without a substrate. FIG. 1A schematically shows a workflow diagram of the method. In process 106, a linker and optionally a polymerizable molecule, e.g., a nucleic acid molecule, are provided, and a monomer of the polymeric analyte is contacted with the linker, thereby generating a monomer-linker complex. In some instances, the linker is pre-tethered to the polymerizable molecule; alternatively, the linker and the polymerizable molecule may be provided separately and joined at any useful or convenient step. In process 112, the monomer-linker complex is coupled to a capture moiety. Coupling of the monomer-linker complex to the capture 105 may be mediated by the polymerizable molecule (e.g., via ligation, hybridization of nucleic acid molecules comprised by the polymerizable molecule and the capture moiety). Optionally, the monomer-linker complex and the capture moiety may be covalently linked together, e.g., using chemical (e.g., click chemistry) or enzymatic (e.g., a ligase, polymerase) approaches. In process 113, the monomer may be cleaved from the polymeric analyte, thereby generating a modified monomer that comprises the cleaved monomer, the linker, and the polymerizable molecule (if present). In process 125, any combination of processes 106, 112, and 113 are repeated to generate additional modified monomers in a cyclic fashion. Each additional modified monomer from a cycle may be coupled to that of the previous cycle, thereby generating a stacked plurality of modified monomers. In process 130, the modified monomer or a modified monomer of a stacked plurality of modified monomers is detected.

FIG. 1B schematically shows an example of the workflow outlined in FIG. 1A. For illustration purposes, the workflow operations are shown for a single polymeric analyte 103; however, it will be appreciated that the workflow operations of FIGS. 1A-1C may be performed on all or a subset of the polymeric analytes in parallel. In workflow 100a, a polymeric analyte 103 (e.g., a peptide) and a capture moiety 105 are provided, which optionally are coupled to a substrate 101, such as a bead, a flow cell, a particle, etc. The capture moiety 105 may comprise a first nucleic acid molecule (e.g., DNA), optionally comprise a barcode sequence. In some examples, the polymeric analyte 103 is coupled to the capture moiety 105, and the capture moiety 105 may subsequently be coupled to the substrate 101. Alternatively, the capture moiety 105 may be precoupled to the substrate 101 and then coupled to the polymeric analyte 103. In process 106, a linker 109 and a polymerizable molecule 111 (e.g., a nucleic acid molecule), are provided. In some instances, the linker 109 is pre-tethered to the polymerizable molecule 111; alternatively, the linker 109 and the polymerizable molecule 111 may be provided separately and joined at any useful or convenient step. In process 106, the linker 109 may couple to a monomer, e.g., an amino acid (e.g., NTAA) of the polymeric analyte 103 (e.g., a peptide) to generate a monomer-linker complex. In process 112, the monomer-linker complex may couple to the capture moiety 105, thereby generating a monomer-capture moiety complex. Coupling of the monomer-linker complex to the capture moiety 105 may be mediated by the polymerizable molecule 111, e.g., via ligation, polymerization, or hybridization. Optionally, the monomer-linker complex and the capture moiety 105 may be covalently linked together (e.g., the polymerizable molecule 111 may be covalently linked to the capture moiety 105), using chemical (e.g., click chemistry) or enzymatic (e.g., a ligase, polymerase) approaches. Alternatively, or in addition to, the polymerizable molecule may comprise a first sequence that is complementary and may hybridize to a second sequence of the capture moiety 105 (not shown), or the polymerizable molecule may be linked to the capture moiety 105 via a splint or bridge molecule, which may comprise sequences that are complementary to the first sequence of the polymerizable molecule and the second sequence of the capture moiety 105 (not shown).

In some instances, the polymeric analyte 103, the capture moiety 105, or both, may optionally be released or removed from the substrate 101, as shown in process 108. Removing the polymeric analyte 103 and the capture moiety 105 from the substrate may be advantageous to prevent intermolecular or crosstalk reactions among neighboring or adjacent polymeric analytes and the capture moieties associated therewith. Other benefits and advantages may include improving reaction kinetics, e.g., of process 112. The removal of the polymeric analyte 103 and capture moiety 105 from the substrate may occur at any useful or convenient step, e.g., prior to, during, or subsequent to processes 106, 112, or 113. In some instances, the removed polymeric analyte 103 and the capture moiety 105 may optionally be re-attached to the substrate 101, as shown in process 116, which may occur at any useful or convenient step, e.g., subsequent to process 112. Re-attachment of the polymeric analyte 103 and the capture moiety 105 to the substrate may be useful, for example, in facilitating iteration of one or more processes, for purification purposes, or for preventing loss of the polymeric analyte 103 in solution.

Processes 108 and 116 may be mediated by reversible attachment and release strategies. For example, the polymeric analyte 103 or the capture moiety 105 may comprise a restriction site that can be cleaved using a restriction enzyme in process 106 but can be rehybridized or ligated in process 116. Alternatively, the substrate, capture moieties, or polymerizable molecule 111 may comprise a releasable or labile moiety, as described elsewhere herein.

In process 113, the monomer may be cleaved from the polymeric analyte 103, thereby providing a modified monomer that comprises the cleaved monomer; the modified monomer may comprise the cleaved monomer coupled to the linker 109, the polymerizable molecule, the capture moiety 105, or a combination thereof (e.g., the modified monomer may comprise the cleaved monomer, the linker, and the polymerizable molecule). In process 125, any one or combination of processes 106, 108, 112, 116, and 113 may be repeated or iterated, thereby generating additional modified monomers.

Any of the operations, e.g., 106, 112, and 113, and 108 and 116 if applicable, may be iterated and repeated (as shown in process 125) any number of times (“rounds” or “cycles”) using additional linkers 109 and polymerizable molecules (e.g., nucleic acid molecules optionally comprising cycle/round information), and tethering the additional polymerizable molecules together (e.g., tethering an additional polymerizable molecule to the polymerizable molecule of the previous cycle). The polymerizable molecules may be coupled using any useful approach, e.g., chemical or enzymatic ligation, linkers, polymerization, or other approach. For instance, the polymerizable molecules may comprise nucleic acid molecules, which may be coupled together using a nucleic acid reaction, e.g., a ligation (e.g., chemical ligation using click chemistry, enzymatic ligation using a ligase), or extension reaction (e.g., using a polymerase). Multiple rounds may be performed until all or a subset of the monomers in the polymeric analyte 103 are cleaved and tethered together. In some instances, processes 106, 112, and 113 may be iterated to generate a stacked plurality of modified amino acids 123 comprising a set of cleaved monomers, e.g., a concatenated set of modified monomers that each comprise a cleaved monomer and a polymerizable molecule coupled thereto (e.g., via a linker 109). The stacked plurality of modified amino acids 123 may comprise a stacked set of polymerizable molecules (e.g., via coupling of the polymerizable molecule from a second round to the polymerizable molecule from a first round and coupling of the polymerizable molecule from the third round to that of the second round, and so on) from the individual modified monomers. The stacked plurality of modified amino acids 123 may comprise polymerizable molecules that are coupled or concatenated in a linear fashion (e.g., polymerizable molecules that form a linear polymerizable molecule “backbone”), or in a non-linear fashion (e.g., random or semi-random arrangement, branched coupling). The stacked plurality of modified amino acids 123 may be circularized; for example, the stacked plurality of modified amino acids 123 forming a linear polymerizable backbone may be joined at the ends to form a circularized product. In some instances, the stacked plurality of amino acids 123 may be cleaved from the substrate. For example, the capture moiety 105 or the polymerizable molecule (e.g., linking nucleic acid molecule 111) may comprise a restriction or cleavable site that can be cleaved upon addition of the proper cleaving reagent, e.g., a restriction enzyme, a reducing agent (for disulfide bonds), etc. Alternatively, or in addition to, the stacked set of polymerizable molecules may be subject to amplification to generate amplicons of the polymerizable molecules coupled to or comprised by the stacked plurality of modified amino acids. The stacked plurality of modified amino acids 123 may be subjected to analysis or characterization, e.g., by contacting the stacked plurality of modified amino acids 123 with a library of binding agents, which can bind to their respective monomer targets (e.g., an amino acid type), and detecting the binding agents, or by using a nanopore sequencer, as described elsewhere herein.

In some instances, the polymerizable molecule comprises temporal information on the cycle in which it is provided; as such, the temporal information may be used for reconstructing the sequence of the peptide or for quality control. For example, referring to FIG. 1B, if a particular cycle number is missing, then it can be inferred that an amino acid is missing or was not present in the peptide, that cleavage of the amino acid did not occur, the linker or polymerizable molecule did not successfully couple to the monomer, or other error. For reconstruction purposes, e.g., referring to FIG. 1B, the presence of a temporal (e.g., round or cycle number) barcode and a peptide-identifying barcode can be used to attribute a particular identified modified amino acid to the order or position (using the temporal barcode) in which it occurs in a specific peptide (using the peptide-identifying barcode). In some instances, temporal information may be provided separately. For example, prior to, during, or subsequent to coupling of a polymerizable molecule 111 to the capture moiety, a temporal barcode may be provided that can couple to the polymerizable molecule 111 or to the capture moiety 105, or a combination thereof. The temporal barcode may comprise any useful agent, including a nucleic acid molecule, a peptide, a lipid, a carbohydrate, an enzyme (e.g., a chromogenic or fluorogenic enzyme) or a ribozyme or DNAzyme, a fluorophore, a dye, an intercalating agent, a dideoxynucleotide, a fluorescent nucleic acid molecule or nucleotide, a radioisotope, a mass tag, or other detectable label that can indicate the time or cycle (or iteration) number in which it is provided. In some instances, the temporal barcode comprises a cycle-specific nucleic acid barcode molecule, which can couple to the polymerizable molecule 111 or to the capture moiety 105. The temporal barcode may comprise any additional useful functional sequences, e.g., primer sites, sequencing sites, restriction sites, abasic or cleavable sites, etc. In some instances, the temporal barcode may comprise an amplification site that allows for bridge amplification of the temporal barcode and optionally, the coupled polymerizable molecules, to other capture or polymerizable molecules.

In some embodiments, the disclosed methods may comprise sequencing both the stacked plurality of modified amino acids and the polymerizable molecule (e.g., an oligonucleotide). In some instances, as described elsewhere herein, the oligonucleotide may comprise one or more barcode sequences. In some instances, at least one of the one of more barcode sequences may comprise error correcting barcodes-barcode sequences that facilitate the identification and correction of sequencing errors, e.g., erroneous base calls. Non-limiting examples of error-correcting barcode designs include Hamming codes, Levenshtein codes, Reed-Solomon codes, etc. Hamming codes, for example, are a family of linear error-correcting codes. Linear codes are codes for which any linear combination of codewords (corresponding to specific nucleic acid barcodes in the context of the instant disclosure) in a codebook comprising the complete set of codewords is also a codeword that can be used to detect and correct for sequencing errors. A codeword of length n can be used to transmit information blocks containing n symbols. The [7,4,3] Hamming code, for example, can be used to represent 4-bit messages using 7-bit codewords. Any two distinct codewords of the complete set of [7,4,3] Hamming codewords differ in at least three bits (the minimum pairwise Hamming distance for this Hamming code) such that up to two errors per codeword can be detected, while a single error (corresponding to a single bit substitution) can be corrected. In general, for each integer r≥2, there is a Hamming code with block length n=2^{r}−1 and message length 1=2^{r}−r−1, where 1 is the number of bits of useful information. A set of equal length codewords comprising a minimum pairwise Hamming distance d=2k+1 (i.e., d is the number of positions at which corresponding bits in the two codewords differ) allows for the correction of up to k errors.

Alternatively, or in addition, Levenshtein or Reed-Solomon codes may be used. Levenshtein codes are similar to Hamming codes but allow for variable length codewords. The pairwise Levenshtein distance between two codewords is the number of single bit edits, e.g., insertions, deletions, or substitutions) required to transform one codeword into the other. Reed-Solomon codes are a group of error-correcting linear block codes that have the property of being a maximum distance separable (MDS) code. Reed-Solomon codes with parameters (n, k), where n is the block length and k is the message length, and a pairwise separation distance of d=n−k+1 can be used to detect—but not correct—up to t=n−k errors, and can be used to correct up to t/2 errors (see, e.g., I. Reed and G. Solomon (1960), “Polynomial Codes Over Certain Finite Fields”, J Soc. Indust. Appl. Math. 8 (2): 300-304, which is incorporated by reference herein in its entirety).

In the context of sequencing, errors can be detected when a detected barcode sequence read (corresponding to a codeword consisting of, e.g., a different 4-bit block for each of the four oligonucleotide bases) doesn't match one of the codewords corresponding to a known barcode sequence as a result of, e.g., a base calling error. The erroneous barcode sequence can then be corrected by replacing the detected barcode sequence with one corresponding to a known codeword that has the highest probability of having been the correct codeword (e.g., the known codeword that can be generated from the erroneous detected codeword using the fewest bit flips). Codebooks comprising sets of codewords corresponding to nucleic acid barcode sequences can be designed to provide error correction capability to correct for, e.g., 1, 2, 3, 4, or 5 errors (e.g., base-calling errors corresponding to substitution errors in the corresponding binary codewords). Barcode design and algorithms for generating and decoding barcodes to support error detection and correction in the context of nucleic acid sequencing applications are described in, e.g., International Patent Publication WO 2022/060889, which is incorporated herein by reference in its entirety.

It will be appreciated that the modified amino acid may comprise other additional modifications that are not depicted, such as posttranslational modifications or chemical modifications (e.g., protecting groups), described elsewhere herein. In some instances, the modified amino acid comprises a derivatized amino acid. For instance, the linker may comprise a PITC or ITC moiety as the amino acid reactive group, and upon conjugation of PITC or ITC to an amino acid (e.g., NTAA) of a peptide under mildly basic conditions, a phenylthiocarbamoyl (PTC) or thiocarbamoyl derivative of the amino acid is generated. The PTC-derivatized or thiocarbamoyl-derivatized amino acid may be treated with acid (e.g., TFA or a Lewis acid) to generate a cleaved cyclic 2-anilino-5(4)-thiazolinone (ATZ)-derivatized or thiazolinone-derivatized amino acid, leaving a new N-terminus on the remaining peptide. The ATZ-derivatized or thiazolinone-derivatized amino acid may be converted to a phenylthiohydantoin (PTH) or thiohydantoin derivative or PTC or thiocarbamoyl derivative.

It will also be appreciated that the polymerizable molecules and capture moieties described herein may comprise other molecule types, e.g., peptides, lipids, carbohydrates, polymers (both naturally occurring and synthetic), or a combination thereof. For instance, referring again to FIGS. 1A-1B, the capture moiety 105 and the polymerizable molecule 111 may each comprise a peptide, optionally comprising a peptide barcode sequence. In such examples, process 112 (coupling of the monomer-linker complex to the capture moiety) may be mediated using a peptide enzyme such as sortase A.

Iteration: In some instances, one or more of the operations described herein may be iterated or repeated. Iteration of the operations may allow for sequential processing, analysis, or identification of the individual monomers of the polymeric analyte, which can allow for reconstruction of the entire polymeric analyte. For example, referring to FIGS. 1A-1B, the operations of the workflow may be conducted to generate a modified amino acid sequentially for each terminal monomer (e.g., NTAA) of the polymeric analyte (e.g., peptide). In some instances, the individual modified amino acids generated from each cycle or round may be combined, e.g., via hybridization or ligation of the polymerizable molecules to generate the stacked plurality of modified amino acids. As such, the stacked plurality of modified amino acids may comprise a plurality of polymerizable molecules from multiple rounds or cycles. In some instances, the polymerizable molecule of the second (or third, fourth, fifth, . . . nth) cycle may be configured to only couple to the first (or second, third, fourth, . . . n-1th) polymerizable molecule. For example, the first cycle polymerizable molecule may comprise a unique binding sequence that is absent on the capture moiety of the substrate, and to which the second cycle polymerizable molecule can bind. Accordingly, the second cycle polymerizable molecule may only bind to the first cycle polymerizable molecule and not to any of the capture moieties. In the event that an amino acid is missed during a cycle (a “null” event, e.g., not cleaved, not coupled to the linker or polymerizable molecule, etc.), a bridging polymerizable molecule may be provided that encodes for a null event but comprises the unique binding sequence, such that subsequent rounds may continue, even if a null event occurs. Alternatively, the polymerizable molecules across cycles or rounds may comprise the same binding sequence (e.g., an adapter sequence).

In some instances, a plurality of click chemistry moieties may be used in the polymerizable molecules for different cycles. For instance, during a first cycle, the polymerizable molecule may comprise tetrazine, which may react with a linker provided in the first cycle that comprises TCO. During the second cycle, the polymerizable molecule and the linker may comprise moieties for an orthogonal click chemistry to that of the first cycle, e.g., sulfur fluoride exchange (SuFEx) click chemistry. Accordingly, temporal information may be provided by the different click chemistry moieties, in addition to or alternatively to using cycle barcodes.

Nanopore Sequencing of Stacked Plurality of Modified Amino Acids: In some instances, the stacked plurality of modified amino acids is subjected to nanopore sequencing using a polymerization-based approach. FIG. 1C schematically shows an example of such a polymerization-based nanopore sequencing approach. In workflow 100b, a stacked plurality of modified amino acids 123 is provided. The stacked plurality of modified amino acids 123 may comprise a blocking region (e.g., a hairpin structure, duplex or double-stranded region, a quadruplex (e.g., G quadruplex), a guanine tetrad, or other bulky moiety such as a protein or large cholesterol moiety) 134 at one or both ends that prevents the stacked plurality 123 from escaping a nanopore 131. In some embodiments, the blocking region 134 comprises a self-annealing region that may self-anneal under a set of conditions (e.g., after entering the aperture of the nanopore 131). For instance, the blocking region may comprise a hairpin loop sequence that comprises a self-annealing region; however, prior to translocation, the stacked plurality of modified amino acids may be contacted with a complementary sequence (not shown) that binds to the self-annealing region to form a double-stranded portion. Prior to or during translocation, the complementary sequence may be removed (e.g., by chemical or enzymatic denaturation or application of an electric potential sufficient to shear off the complementary sequence), such that the self-annealing region may self-hybridize, forming a hairpin moiety, once translocated to the trans compartment (not shown). The stacked plurality of modified amino acids 123 may additionally comprise a double-stranded region at one end, which may act as a priming or attachment region for a polymerizing enzyme. In some instances, the double-stranded region may comprise a primer annealed to the stacked plurality of modified amino acids (e.g., adjacent to the blocking region 134); in such instances, the primer may be provided in the reaction mixture. In process 135, a portion of the stacked plurality of modified amino acids 123 may enter (e.g., via application of an electric potential) an aperture of the nanopore 131 and be in an incorporate position (e.g., as shown in process 135). The nanopore 131 may be embedded in a membrane 132 (e.g., a lipid bilayer or solid-state membrane). A polymerizing enzyme 128 (e.g., a DNA or RNA polymerase) is provided (e.g., in the reaction mixture) adjacent to the nanopore 131 on one side of the membrane 132 (e.g., in the cis compartment). The polymerizing enzyme 128 may incorporate a monomer 126 (e.g., nucleotide) adjacent to the double-stranded priming region, thereby generating a partially polymerized stacked plurality of modified amino acids. In process 137, the partially polymerized stacked plurality of modified amino acids may translocate in a first direction (e.g., a cis-to-trans direction), e.g., by application of an electric potential, to a read position (e.g., as shown in process 137). The blocking region 134 or the double-stranded priming region (e.g., at or around the incorporation site) at the first end may prevent the partially polymerized stacked plurality of modified amino acids from fully translocating through the nanopore 131. For instance, in the read position, the stacked plurality of modified amino acids may be positioned such that the double stranded region and the newly incorporated monomer are positioned within the aperture of the nanopore but not able to pass through the narrowest constriction of the nanopore. Prior to, during, or subsequent to the translocation of the partially polymerized stacked plurality of modified amino acids in the first direction, a signal (e.g., current signal, current blockade, impedance, or other signal) is measured at or adjacent to the nanopore 131. In process 139, the partially polymerized stacked plurality of modified amino acids may translocate in a second direction (e.g., a trans-to-cis) direction, e.g., by reversing the applied electric potential. The partially polymerized stacked plurality of modified amino acids may be retained in the aperture of the nanopore by a blocking region at the second end. The incorporate-read cycle of monomer incorporation and bidirectional translocation is repeated N times (where N is an integer), and the signal is continuously or discretely measured at or adjacent to the nanopore 131, allowing for controlled, repeatable positioning of the stacked plurality of modified amino acids within the nanopore and repeat measurements of signals at a plurality of positions along the same stacked plurality of modified amino acids 123. The measured signals can be used to determine an amino acid type of individual modified amino acids comprised by the stacked plurality of modified amino acids.

In some instances, nanopore sequencing of modified amino acids may be performed without a motor protein. FIG. 1D schematically shows an example of a primer-based translocation control approach for nanopore sequencing of a modified amino acid or stacked plurality of modified amino acids. In workflow 100c, a modified amino acid (or stacked plurality of modified amino acids) 123 comprising a nucleic acid molecule (e.g., a single-stranded DNA molecule) is provided. The modified amino acid or stacked plurality of modified amino acids may additionally comprise a blocking region 134 at one or both ends to prevent the molecule from escaping the nanopore 131. For instance, the blocking region may comprise a hairpin loop sequence that comprises a self-annealing region; however, prior to translocation, the stacked plurality of modified amino acids may be contacted with a complementary sequence (not shown) that binds to the self-annealing region to form a double-stranded portion. Prior to or during translocation, the complementary sequence may be removed (e.g., by chemical or enzymatic denaturation or application of an electric potential sufficient to shear off the complementary sequence), such that the self-annealing region may self-hybridize, forming a hairpin moiety, once translocated to the trans compartment (not shown). In process 145, the modified amino acid with a double-stranded region 144 is threaded into the aperture of the nanopore 131 (e.g., embedded in a membrane 132) by application of an electric potential. The stacked plurality of modified amino acids 123 may be contacted with a plurality of primers 142 (e.g., short DNA oligonucleotides of about 2-20 nucleotides in length) that are configured to hybridize to complementary sequences along the nucleic acid molecule 140, thereby generating a plurality of double-stranded regions along the nucleic acid molecule. In process 147, the plurality of primers may hybridize to the nucleic acid molecule 140. In process 149, the modified amino acid translocates through the nanopore in a first direction (e.g., cis-to-trans). As each double-stranded region encounters the constriction of the nanopore, translocation is arrested as the double-stranded region cannot pass through the narrow constriction. The arrested positioning allows for measurement of a signal (e.g., ionic current) of the amino acid portion of the modified amino acid that is positioned in or adjacent to the constriction zone. In process 149, upon application of a sufficient electric force or other denaturant, the double-stranded region is sheared or denatured away, releasing the primer 142 and allowing the next segment of the modified amino acid to advance through the nanopore to the next double-stranded region. This process is repeated for each successive double-stranded region, achieving a stepwise ratcheting of the modified amino acid through the nanopore without the use of a motor protein (e.g., a helicase, polymerase). A signal (e.g., ionic current) is measured at or adjacent to the nanopore either continuously or during each arrested position, yielding a series of signal levels that correspond to the different portions of the modified amino acid.

In some instances, the measured signal is a current signal under an applied voltage. In some instances, the current signal can be measured from or adjacent to the nanopore under a plurality of applied voltages (e.g., a discrete set or gradient of applied voltages). In such instances, in the read position of an incorporate-read cycle, the stacked plurality of modified amino acids may be subject to a first applied voltage and the signal measured. Subsequently, a second applied voltage may be applied and the signal measured. Any useful number of applied voltages may be applied. In some instances, a gradient or variable (e.g., time-varying) voltage may be applied, which may increase calling accuracy, e.g., as described in M. Noakes et al. (2019) Nature Biotechnology. 37, 651-656, which is incorporated by reference herein. Beneficially, measuring signal of the stacked plurality of modified amino acids may allow for obtaining nonredundant information that can be used to further distinguish the individual monomers and modified amino acids.

In some embodiments, the accuracy of identification of an amino acid type is improved by measuring signals under a plurality of applied voltages. For example, measuring a plurality of signals under a plurality of applied voltages may provide a multi-dimensional feature space for amino acid type identification, wherein each applied voltage may elicit a different signal characteristic from the modified amino acid, thereby providing nonredundant information. In some instances, the accuracy of identification using signals from a plurality of applied voltages (e.g., 2, 3, 4, 5 or more voltages) is at least about 5%, at least about 10%, at least about 15%, at least about 20%, at least about 25%, at least about 30%, at least about 40%, or at least about 50% higher than the accuracy of identification using signals from a single applied voltage. In some embodiments, a current signal measured at a first applied voltage may be informative for distinguishing a first pair of amino acid types (e.g., leucine and isoleucine), while a current signal measured at a second applied voltage may be informative for distinguishing a second pair of amino acid types (e.g., glutamic acid and aspartic acid). As such, the combination of signals from multiple voltages may provide complementary information that improves overall amino acid type identification accuracy. In another example, measuring current signals at three different applied voltages (e.g., 40 mV, 60 mV, and 80 mV) may yield multi-dimensional signal data (e.g., mean current, current standard deviation, peak width, peak or maximum current, CV at each voltage) that provides a richer feature space for amino acid type discrimination than measuring at a single voltage alone.

In some instances, the plurality of applied voltages may be applied as a time-varying voltage, e.g., a stepped waveform, a ramp, a sinusoidal waveform, or a combination thereof. In some instances, the applied voltage alternates between a negative voltage (e.g., in the incorporate position) and a positive voltage (e.g., in the read position), as shown in FIG. 1C. In some embodiments, the time-varying voltage is configured to optimize the signal-to-noise ratio for different amino acid types.

In some embodiments, the applied voltage or plurality of applied voltages is in a range of about 10 millivolts (mV) to about 500 mV, about 20 mV to about 200 mV, about 20 mV to about 120 mV, about 30 mV to about 100 mV, or about 40 mV to about 80 mV. In some embodiments, the plurality of applied voltages comprises at least 2, at least 3, at least 4, at least 5, at least 6, at least 7, at least 8, at least 9, or at least 10 different voltage levels. For example, the plurality of applied voltages may comprise a first voltage of about 40 mV, a second voltage of about 60 mV, and a third voltage of about 80 mV.

In some instances, following any useful number of incorporate-read cycles, (e.g., after all or a portion of the stacked plurality of modified amino acids comprises a complementary strand), re-sequencing or consensus sequencing may be performed. Re-sequencing or consensus sequencing may comprise removing the polymerized monomers, and optionally, the double-stranded priming region (e.g., if a complementary primer is used) from the stacked plurality of modified amino acids, thereby restarting the workflow (e.g., 100b). The removal of the polymerized monomers may be performed using any useful technique, e.g., chemical denaturation, heat denaturation, or application of a greater applied voltage (e.g., to force translocation of the fully polymerized stacked plurality of modified amino acids in a direction through the nanopore, thereby shearing the polymerized monomers off against the constriction zone of the nanopore).

Beneficially, re-sequencing or consensus sequencing of the same modified amino acid or stacked plurality of modified amino acids may improve the accuracy of amino acid type identification. By performing multiple rounds of sequencing on the same molecule, signal noise can be averaged out and a consensus identity can be determined with higher confidence. In some embodiments, the re-sequencing is performed at least 2, at least 3, at least 4, at least 5, at least 6, at least 7, at least 8, at least 9, at least 10, at least 15, at least 20, at least 50, or at least 100 times. Each round of re-sequencing may generate a measured signal or a set of measured signals, and the measured signals from the multiple rounds may be aggregated (e.g., averaged, filtered, or weighted) to generate a consensus signal. In some embodiments, the consensus accuracy of re-sequencing for a given amino acid type exceeds about 50%, about 60%, about 70%, about 80%, about 85%, about 90%, about 95%, about 97%, about 99%, or about 99.5%. In some embodiments, the consensus accuracy exceeds 80% for at least 2, at least 5, at least 10, at least 15, or at least 20 distinct amino acid types. As used herein, consensus accuracy refers to the fraction of correct amino acid type identifications when using the aggregated signals from multiple re-sequencing rounds.

In some embodiments, re-sequencing may be performed to generate independent signal measurements of the same modified amino acid or stacked plurality of modified amino acids, and the independent measurements may be combined to improve the signal-to-noise ratio. For instance, a first round of sequencing may produce a first set of measured signals across a plurality of incorporate-read cycles, and subsequent to removal of the polymerized monomers, a second round of sequencing may produce a second set of measured signals. The first and second sets of measured signals may be aligned and combined, e.g., using statistical methods such as averaging, weighted averaging, or maximum likelihood estimation, to yield a consensus identification with higher confidence than either individual round. In some instances, re-sequencing may enable discrimination between amino acid types that are difficult to distinguish in a single sequencing round, e.g., amino acids with similar size or charge characteristics (e.g., leucine and isoleucine, or glutamic acid and aspartic acid).

In some instances, sequencing reads may be assembled using a de novo approach to identify the peptide or protein. For instance, fragmented peptides arising from a common parent protein may be labeled with a common barcode sequence, as described elsewhere herein. In addition, additional barcodes comprising, e.g., cycle number barcodes, unique molecular identifiers (UMIs), spatial barcodes, etc., may be incorporated into the polymerizable molecule during expansion of polymeric analytes (e.g., polypeptides), as described elsewhere herein. Putative peptide reads can thus be assembled based on the measured signals for the modified monomers (e.g., modified amino acids) of the polymeric analyte and the measured signals for the polymerizable molecules comprising barcoded information, e.g., common barcode sequences, spatial barcodes, cycle number barcodes, etc. Erroneous reads may be identified through probabilistic modeling of accuracy of reads, resulting in reconstructed, fragmentary, peptide sequences (contigs) with possible gaps for missed or unidentified rounds/amino acid.

In some instances, e.g., in cases where it is difficult to distinguish between the measurable signals for two monomers of the polymeric analyte (e.g., between two natural and/or non-natural amino acid residues of a polypeptide that are similar in molecular size, geometry, polarity, electrical charge, etc.), it may be advantageous to perform differential labeling using a set of orthogonal, amino acid residue-specific, labeling or modification reagents. For example, a peptide sample may be divided into 2, 3, 4, 5, or more than 5 aliquots, where a first aliquot is left untreated and the remaining sample aliquots are each reacted with a single labeling or modification reagent that is specific for a given amino acid type and which, upon reacting with the given amino acid type, produces a net change in molecular size, geometry, polarity electrical charge, etc., that results in a difference in the measurable signal(s) associated with the given amino acid type. After sequencing each sample aliquot using the methods described elsewhere herein (e.g., workflows 100a, 100b, and 100c), comparison of the sequences obtained for the differentially labeled sample aliquots to that obtained for the untreated control sample may help resolve the identity and/or confirm the identity of one or more amino acid residues in the original polypeptide. Examples of such amino acid-specific labeling or modification reactions for peptides and proteins include, but are not limited to, alkylation of cysteine residues (e.g., using 4-vinylpyridine, iodoacetamide, iodoacetate, chloroacetate), aminoethylation of cysteine residues (e.g., using 2-bromoethylamine), acetylation of serine or threonine residues to form an ester (e.g., using acetyl chloride or acetic anhydride), oxidation of cysteine residues to cysteic acid, reduction of disulfide bridges in cystine residues (e.g., using dithiothreitol, β-mercaptoethanol, or TCEP), etc. Additional non-limiting examples include: (i) reaction of lysine residues with activated esters, sulfonyl chlorides, isothiocyanates, 2-amino-2-methoxyethyl (IME), and squaric acids, as well as reductive alkylation using aldehydes in the presence of sodium borocyanohydride; (ii) reaction of tyrosine residue side-chains with diazobenzene compounds or a combination of aldehyde and aniline derivatives; (iii) reaction of tryptophan residues with an organic radical compound, 9-azabicyclo[3.3.1]nonane-3-one-N-oxy (ketoABNO) together with NaNO₂; and (iv) alkylation of methionine residues using alkyl halide reagents to introduce a broad range of functional groups; as described in S. Sakamoto and I. Hamachi (2019), “Recent Progress in Chemical Modification of Proteins”, Analytical Sciences 35:5-27, which is incorporated herein by reference in its entirety. Orthogonal labeling reactions and/or site-specific modifications may be performed prior to, during, or after performing the expansion reactions described elsewhere herein (provided that the required reaction conditions are compatible), and may be directed to labeling or modifying any of the monomers (e.g., amino acid residues) within a polymeric analyte (e.g., a polypeptide), including N-terminal amino acid residues, C-terminal amino acid residues, or internal amino acid residues.

It will be appreciated that although reference to single modified amino acids and stacked pluralities of modified amino acids are described herein, the methods and systems described herein may apply to both the singular and the plurality of modified amino acids. For example, any of the operations of workflow 100b may be performed on individual modified amino acids, rather than a stacked plurality of modified amino acids. Similarly, any operations performed on a modified amino acid may also be performed on a stacked plurality of modified amino acids.

It will also be appreciated that while reference to a first direction and a second direction are exemplified as cis-to-trans and trans-to-cis directions, respectively, that either direction is possible using the methods described herein. For instance, the incorporation position using the polymerizing enzyme may occur in the trans compartment, and the translocation in the first direction may occur in a trans-to-cis direction. Similarly, the modified amino acid may be detected (signal measured) while translocating in the trans-to-cis direction.

Detection Using Binding Agents: In some instances, detecting comprises using monomer-specific binding agents to recognize and bind the cleaved monomers. In some embodiments, the monomer-specific binding agents are used for direct or indirect detection; for example, the binding agents may comprise a detectable label (e.g., fluorophore, mass tag, radioisotope) that can be detected directly, or the binding agent may comprise a polymerizable molecule with encoded information, which may be transferred via coupling to or copying of the encoded information onto the capture moiety or an additional polymerizable molecule. In some instances, the additional polymerizable molecule or the capture moiety is located adjacent to the polymeric analyte. Alternatively, or in addition to, the binding agents may be used to sort the cleaved monomers, e.g., into separate partitions or compartments for downstream labeling, e.g., with identifying barcode molecules. The operations may be iterated or repeated any number of times to obtain information on all or a subset of the monomers of the polymeric analyte and optionally, the sequence of the monomers relative to the polymeric analyte. The information may be read out from the polymerizable molecules using, for example, conventional next-generation sequencing or nanopore sequencing approaches. Beneficially, by cleaving the monomer from the polymeric analyte, the monomers are removed from the adjacent monomers, and binding agents can specifically bind to individual monomers without the influence of the adjacent surrounding monomers. As such, the methods disclosed herein enable more accurate molecular identification and polymeric sequencing, which has applications in diagnosing disease, monitoring protein dynamics or protein interactions, single-cell proteomics, developing or characterizing therapeutics, and more.

Methods of the present disclosure for processing a polymeric analyte comprising a plurality of monomers may comprise cleaving of a monomer of the polymeric analyte and coupling the monomer to a capture moiety (e.g., coupled to a substrate, the polymeric analyte, or provided in solution) for subsequent processing or analysis. In one example, a method of the present disclosure may comprise: providing (i) a polymeric analyte comprising a plurality of monomers and (ii) a capture moiety; coupling a monomer of the plurality of monomers to the capture moiety to generate a monomer-capture moiety complex; cleaving the monomer; contacting the cleaved monomer-capture moiety complex with the binding agent; and coupling a first polymerizable molecule to a second polymerizable molecule or to the capture moiety. In some instances, the first polymerizable molecule is coupled to the binding agent and comprises information about the binding agent, such as the identity of the binding agent or its cognate molecule, which information may be transferred to the second polymerizable molecule or to the capture moiety. In some instances, the capture moiety may be a copy or identical molecule as the second polymerizable molecule. Alternatively, or in addition to, the binding agent may be used to sort a mixture of cleaved monomers by identity or type, and subsequent to sorting, an identifying label or barcode that identifies the monomer type may be coupled to the capture moiety. Example methods and systems of such processing approaches and systems are described in U.S. Pat. No. 11,499,979, International Pat. Pub. No. WO/2023/114732, International Pat. Pub. No. WO/2023/196642, and U.S. patent application Ser. No. 18/740,088, filed Jun. 11, 2024, and International Pat. Pub. No. 2024/178395, each of which is incorporated by reference herein in its entirety.

An example workflow of analyzing a polymeric analyte may include providing a polymeric analyte, sequentially disassembling the polymeric analyte into individual monomers via contacting with capture moieties and cleavage, and contacting the individual cleaved monomers complexed with the capture moieties with binding agents comprising polymerizable molecules that identify or encode for the binding agents. The polymerizable molecule of a binding agent is coupled to an additional polymerizable molecule, and the monomer is cleaved from the capture moiety or blocked to prevent downstream recognition from additional binding agents.

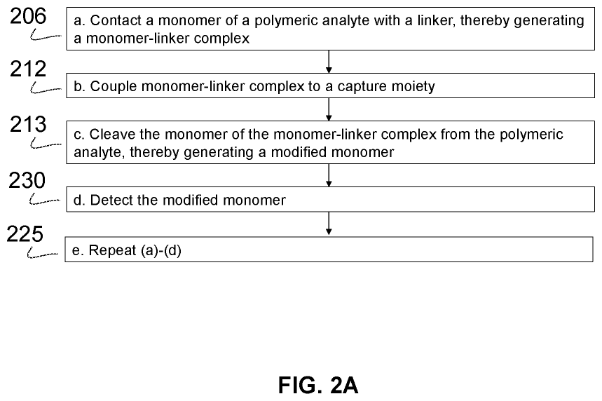

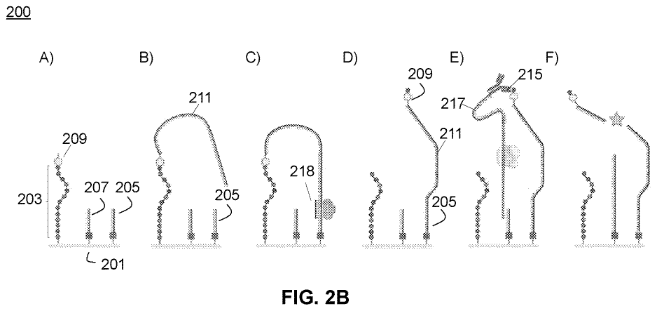

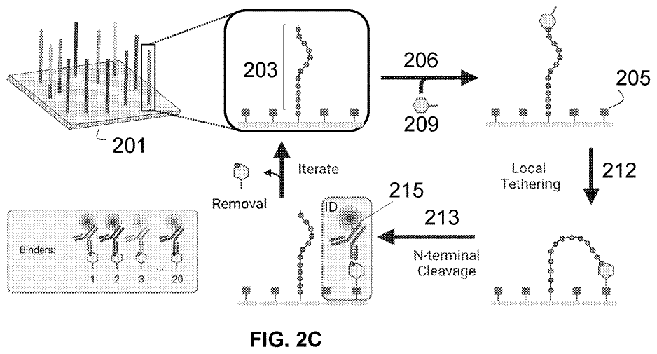

FIG. 2A shows an example workflow of processing and analyzing a polymeric analyte. In process 206, a monomer (e.g., a terminal monomer such as a terminal amino acid) of the polymeric analyte is contacted with a linker, thereby generating a monomer-linker complex. In process 212, the monomer-linker complex is coupled to a capture moiety. In process 213, the monomer is cleaved from the polymeric analyte, thereby generating a modified monomer. In process 230, the modified monomer is detected, e.g., using monomer-specific binding agents. In process 225, any one or combination of processes 206, 212, 213, and 230 is repeated, thereby sequencing the polymeric analyte. In some instances, process 230 is performed after process 225.

FIG. 2B schematically shows an example workflow of analyzing a polymeric analyte. In workflow 200, a polymeric analyte 203 is provided, sequentially disassembled into individual monomers via contacting with linkers, coupling to capture moieties, and cleavage of the monomers from the polymeric analyte, and the individual cleaved monomers are contacted with binding agents comprising polymerizable molecules that identify or encode for the binding agents. The polymerizable molecule of a binding agent is coupled to an additional capture moiety, e.g., an additional polymerizable molecule, and the monomer is cleaved from the capture moiety or blocked to prevent downstream recognition from additional binding agents. In FIG. 2B Panel A, a substrate 201 is coupled to a polymeric analyte 203 (e.g., a peptide to be sequenced), a capture moiety 205 (e.g., a nucleic acid molecule), and an additional capture moiety 207 (e.g., an additional nucleic acid molecule). In some instances, the capture moiety 205 and the additional capture moiety 207 are identical molecules (e.g., comprise the same sequence). The polymeric analyte may be contacted with a bifunctional linker 209, which comprises a terminal monomer-coupling group (e.g., an amino acid-reactive group) and a click chemistry moiety (e.g., azide). In some instances, the bifunctional linker 209 couples to a terminal monomer (e.g., terminal amino acid such as the N-terminal amino acid (NTAA)). In FIG. 2B Panel B, a polymerizable molecule 211 comprising a click chemistry moiety (e.g., alkyne) is reacted with the bifunctional linker 209 and covalently linked. In some instances, the polymerizable molecule 211 and the bifunctional linker 209 are provided pre-coupled (not shown). In FIG. 2B Panel C, the polymerizable molecule 211 is coupled to the capture moiety 205, thereby generating a monomer-capture moiety complex. The coupling may be mediated by hybridization of the polymerizable molecule 211 to the capture moiety 205 (hybridization not shown), or by using a splint oligonucleotide 218 comprising sequences complementary to a sequence of the polymerizable molecule 211 and the capture moiety 205. In some instances (not shown), the polymerizable molecule 211 comprises a self-splinting sequence, such that the polymerizable molecule 211 may couple to the capture moiety 205 in the absence of a separate splint molecule. A ligase may be used to covalently link the polymerizable molecule 211 to the capture moiety 205. Alternatively, the polymerizable molecule 211 may comprise a first reactive moiety (e.g., click chemistry moiety not shown) that can react with a second reactive moiety (not shown) of the capture moiety 205. In FIG. 2B Panel D, the system is subjected to conditions sufficient to cleave the terminal monomer (e.g., amino acid) from the polymeric analyte 203 (e.g., peptide), thereby generating a modified monomer that is complexed to the capture moiety. The conditions may include performing an Edman degradation reaction. The cleavage of the monomer from the polymeric analyte results in a modified monomer comprising the cleaved monomer, bifunctional linker 209, polymerizable molecule 211, and is coupled to the capture moiety 205. In FIG. 2B Panel E, a binding agent 215 (e.g., antibody) comprising another polymerizable molecule 217 (e.g., nucleic acid molecule) is provided. The binding agent 215 may be specific or partially specific to the monomer (e.g., to an amino acid type) or to the modified monomer (e.g., monomer-linker complex). The polymerizable molecule 217 of the binding agent may comprise information (e.g., a nucleic acid barcode molecule) on the identity of the binding agent or the specific monomer (e.g., single amino acid) to which the binding agent binds. The polymerizable molecule 217 of the binding agent may comprise additional sequences, such as a barcode sequence, UMI, restriction site, transposition site, a temporal barcode sequence to represent a cycle or iteration number, or other functional sequence. The polymerizable molecule 217 of the binding agent may couple to the additional capture moiety 207 that is coupled to the substrate 201. In some instances, an extension reaction may be performed (e.g., using a polymerase), to copy the sequence of the polymerizable molecule 217 of the binding agent to the additional capture moiety 207 that is coupled to the substrate 201, thereby encoding or barcoding the information from the polymerizable molecule 217 onto the additional capture moiety 207. Alternatively, the polymerizable molecule 217 of the binding agent may be ligated to the additional capture moiety 207, either chemically (e.g., via complementary click chemistry) or enzymatically (e.g., using a ligating enzyme, ribozyme or DNAzyme). Optionally, the polymerizable molecule 217 of the binding agent may be cleaved from the binding agent 215 (not shown), e.g., the polymerizable molecule 217 of the binding agent may comprise a releasable or cleavable moiety. In FIG. 2B Panel F, the monomer may be decoupled (e.g., removed or cleaved) from the capture moiety 205. For example, the monomer, bifunctional linker 209, and all or a portion of the linking nucleic acid molecule 211 may be cleaved (depicted as a star). The cleavage may be performed chemically, mechanically, or enzymatically. In an example of enzymatic cleavage, the linking nucleic acid molecule 211 may comprise a restriction site or other cleavage site (e.g., a uracil), and cleavage occurs by introduction of a restriction enzyme or cleaving enzyme (e.g., uracil DNA glycosylase) to cleave the restriction/cleavage site. Alternatively, the cleaved monomer-capture moiety complex may be blocked with a blocking agent (not shown). The workflow may then be iterated or repeated to process all or a portion of the polymeric analyte 203.

Any of the processes, e.g., as shown in 206, 212, 213, 230, 225 of FIG. 2A or FIG. 2B Panels A-F may be iterated and repeated any number of times (“rounds”) using additional linkers 209 and polymerizable molecules (optionally comprising cycle/round information), and tethering the additional polymerizable molecules together (e.g., tethering an additional polymerizable molecule to the polymerizable molecule of the monomer-capture moiety complex). Multiple rounds may be performed until all or a subset of the monomers in the polymeric analyte 203 are cleaved and tethered together. In some instances, processes 206, 212, and 213 may be iterated to generate a stacked plurality of modified monomers e.g., a concatenated set of monomer-linker-polymerizable molecule complexes, which are linked to one another by the polymerizable molecules from each cycle. Alternatively, or in addition to, one or more modified monomers may be attached to yet another capture moiety, and encoding of the polymerizable molecule of the binding agent may occur on another adjacent capture moiety.

Identification of polymerizable molecules: The polymerizable molecules or molecules comprising the polymerizable molecules (e.g., a stacked plurality of modified amino acids or a concatenated set of monomer-linker-polymerizable molecule complexes) described herein may be subjected to sequencing to determine the identity of the individual monomers (e.g., sequencing a stacked plurality of modified amino acids using a nanopore; sequencing polymerizable molecules comprising barcodes that encode for individual amino acids).

Alternatively, or in addition to, the polymerizable molecules may be subject to further processing, e.g., amplified (e.g., using nucleic acid amplification approaches such as polymerase chain reaction (PCR), isothermal amplification, ligation-mediated amplification, transcription-based amplification, etc.) to generate amplicons for sequencing. Amplification may be performed, for example, using the capture moieties or polymerizable molecules as primer binding sites.

Any number of useful preparation operations for sequencing may be performed, such as purification or enrichment, cleanup, nucleic acid reactions (e.g., ligation, extension, amplification, tagmentation, restriction enzyme cleavage), fragmenting, barcoding, addition of adapters, enzymatic treatment, etc. In some instances, the polymerizable molecules, or the substrates comprising the polymerizable molecules, may be filtered based on any useful characteristic or properties. Filtering based on a characteristic or property may achieve higher accuracy or less noise by removing poor quality molecules or enriching for higher quality polymerizable molecules prior to sequencing. For example, polymerizable molecules or substrates (e.g., beads or particles) containing the polymerizable molecules may be filtered by size or length, quantity, presence of particular sequences (e.g., primer sequences, sequences of interest), GC content, polarity, polarization, birefringence, fluorescence (or other optical property), anisotropy, charge, secondary structure (e.g., hairpins, guanine tetrads, G-quadruplexes), Raman spectroscopy, nuclear magnetic resonance (NMR), mass spectrometry, surface plasmon resonance (SPR), circular dichroism, ultraviolet-visible (UV-Vis) spectroscopy, infrared (IR) spectroscopy, electrochemical properties, thermal stability, or other useful metric, characteristic, or property or combinations thereof. Such filtration or enrichment may be performed using any suitable approach, e.g., affinity or hybridization approaches (e.g., bead based affinity sequences or hybridization assays, which can enrich particular sequences), chromatography, size-based filtration, electrophoresis, electrofocusing, optoelectronics, digital fluidics, magnetic activated sorting, fluorescence activated sorting, flow cytometry, or other suitable technique.

Sequencing may be performed using any suitable nanopore, e.g., Oxford Nanopore Technologies, Genia Technologies, NobleGen, or Quantum Biosystem, or other sequencing and next generation sequencing systems, e.g., Illumina, BGI, Qiagen, ThermoFisher, PacBio, and Roche, including formats such as parallel bead arrays, sequencing by synthesis, sequencing by ligation (e.g., SOLID), capillary electrophoresis, electronic microchips, “biochips,” microarrays, parallel microchips, single-molecule arrays, and Sanger sequencing, as is described elsewhere herein.

The nanopore sequencer may comprise an array of nanopore unit cells. In some embodiments, each nanopore unit cell of the array of nanopore unit cells is individually and operably coupled to a controller. The controller may be configured to control one or more sequencing operations in the array of nanopore unit cells, including, e.g., actuating a nanopore unit cell, signal detection from a nanopore unit cell, or accessing or controlling nanopore sensors in the array. In some embodiments, the nanopore sequencer may further comprise a computer that is operably connected to the nanopore sequencer. In some embodiments, the nanopore sequencer may further comprise a data storage and computing resource, such as a network or cloud, that may be operably connected to the nanopore sequencer and the computer. In some embodiments, the controller can be implemented in software and/or hardware. The controller can be implemented by electronic hardware, computer software, or combinations thereof, for example, using a processor configured with specific instructions, a digital signal processor (DSP), an application-specific integrated circuit (ASIC), a field programmable gate array (FPGA) or other programmable logic device, discrete gate or transistor logic, discrete hardware components, or any combination thereof.

In some embodiments, the system further comprises a voltage source configured to apply a voltage or a plurality of voltages across the nanopore. The voltage source may be configured to apply a direct current (DC) voltage, an alternating current (AC) voltage, or a combination thereof. The voltage source may be configured to apply a time-varying voltage waveform, e.g., a stepped voltage, a ramped voltage, a sinusoidal voltage, or a pulsed voltage. In some embodiments, the system further comprises a measurement circuit configured to measure an ionic current through the nanopore. The measurement circuit may comprise an amplifier (e.g., a transimpedance amplifier), an analog-to-digital converter, or a combination thereof. In some embodiments, a sampling rate of the measurement circuit is at least about 100 Hz, at least about 500 Hz, at least about 1 kHz, at least about 5 kHz, at least about 10 kHz, at least about 25 kHz, at least about 50 kHz, at least about 100 kHz, or at least about 1 MHz. In some embodiments, the sampling rate is in a range of about 1 kHz to about 100 kHz. Higher sampling rates may provide improved temporal resolution and improved signal quality. In some embodiments, the system further comprises a processor (e.g., a central processing unit (CPU), a graphics processing unit (GPU), a field-programmable gate array (FPGA), or an application-specific integrated circuit (ASIC)) configured to determine an amino acid type comprised by the modified amino acid based on the measured ionic current. The processor may execute one or more algorithms, e.g., a machine learning model, a hidden Markov model, a neural network, a k-mer decoding model, or other computational algorithm, to identify the amino acid type from the measured signal or plurality of measured signals.

In some embodiments, the recognition zone of a nanopore (such as an MspA nanopore or engineered variant thereof) may include a constriction region, which may be a sensitive region for amino acid discrimination since the constriction is where the largest voltage drop occurs as it presents the largest resistance between the cis and trans electrodes. In some embodiments, the nanopore recognition zone may span a length longer than the height of a single amino acid or a single nucleotide of a polymerizable molecule threading through the nanopore, and therefore a current signal that a nanopore generates may be dependent on more than one amino acid or nucleotide simultaneously occupying the recognition zone. For example, the current signal generated by the nanopore may depend on 2, 3, 4, 5, 6, 7, 8, 9, 10 or more nucleotides. These groups of nucleotides may be termed a ‘k-mer.’ A signal associated with the identity of one or more nucleotides and/or amino acids may be decoded from measured nanopore signals by reference to a look-up table or model that maps each k-mer instance to an associated current signal. Beneficially, some of the methods provided herein, including intramolecular expansion for generation of modified amino acids and stacked pluralities of modified amino acids, simplify k-mer deconvolution and amino acid identification, as the spacing between amino acids along the polymerizable molecule backbone is tunable (e.g., separated by a nucleic acid backbone comprising at least 1, at least 2, at least 3, at least 4, at least 5, at least 6, at least 7, at least 8, at least 9, at least 10, at least 20, at least 30, at least 40, or at least 50 nucleotides or greater) such that only a single amino acid is present in the constriction region of the nanopore at a given instant.

In some embodiments, each nanopore unit cell of the nanopore sequencer may include a membrane and a nanopore disposed in or through the membrane. The membrane may be formed from any suitable natural and/or synthetic material. The membrane may be a non-permeable or semi-permeable material. In an example, the membrane includes a bilipid layer or a block copolymer structure. In some embodiments, the nanopore is a hollow structure extending through the membrane. In one embodiment, a protein MspA may be inserted into a pre-formed membrane (e.g., formed of block copolymers or lipids). The membrane separates each nanopore unit cell into a cis compartment (or cis well) and a trans compartment (or trans well). The modified amino acid or stacked plurality of modified amino acids may translocate from the cis compartment, relative to the nanopore, to the trans compartment, or vice versa.

In some embodiments, a cis electrode is associated with the cis compartment and a trans electrode is associated with the trans compartment. The electrodes may be used to apply a voltage across the nanopore, thereby driving ionic current flows through the nanopore and exerting an electric force on the modified amino acid or stacked plurality of modified amino acids. In some embodiments, the cis electrode may be a silver/silver chloride (Ag/AgCl) electrode, a gold (Au) electrode, a platinum (Pt) electrode, a carbon (C) electrode, or other suitable electrode material. In some embodiments, the trans electrode may similarly be a silver/silver chloride (Ag/AgCl) electrode or other suitable electrode material. A current detector may be used to measure the ionic current through the nanopore, and the detected signal may be transmitted to the sequencer controller. An electrolyte may be filled in the cis well and the trans well. The electrolyte may be any electrolyte that is capable of dissociating into counter ions (a cation and an associated anion). In some embodiments, the electrolyte may be a potassium-containing electrolyte (e.g., potassium chloride (KCl)) or a sodium-containing electrolyte (e.g., sodium chloride (NaCl)).

Sequencing may output the identity of the polymerizable molecules or sequences of polymerizable molecules that are coupled together. For example, subsequent to one or more iterations of workflow 100a, the stacked plurality of modified amino acids may be subjected to sequencing. Similarly, subsequent to one or more iterations of workflow 200, the additional capture moiety 207 may comprise a nucleic acid sequence of the polymerizable molecule 217 of the binding agent (or complement thereof), or stacks of polymerizable molecules obtained from multiple rounds of binding agents binding to their target monomer or monomer-capture moiety complex (or complements thereof). Sequencing of the additional capture moiety 207 and any molecules coupled thereto may therefore yield sequencing reads that identify the nucleic acid sequence of the polymerizable molecule 217 of the binding agent and the information encoded therein, e.g., the cycle number and the identity of the binding agent or monomer (e.g., one of the 20 proteinogenic amino acids). In instances where the polymerizable molecule 217 of the binding agent comprises a nucleic acid molecule that encodes additional information (e.g., comprises barcode sequences, UMIs, cycle information, spatial information etc.), multiple types of information may be revealed from the nucleic acid sequencing of the additional capture moiety 207.

Sequencing reads may be assembled using a de novo approach to identify the peptide or protein. For instance, fragmented peptides arising from a common parent protein may be labeled with a common barcode sequence, as described elsewhere herein. Putative peptide reads can thus be assembled based on the common barcode sequence, amino acid identity, and if applicable, cycle number. Erroneous reads may be identified through probabilistic modeling of accuracy of reads, resulting in reconstructed, fragmentary, peptide sequences (contigs) with possible gaps for missed or unidentified rounds/amino acid. An alternative option for de novo read reconstruction may employ end-to-end, unsupervised machine learning based reconstruction of peptide reads. This option may employ a Machine Learning Algorithm, which refers to a deep-learning based model that takes as its input NGS sequencing reads associated with a parent protein/peptide barcode, and outputs the likely reconstruction of peptide reads (contigs). Training of the model can be conducted with protein sequencing runs using known protein/peptide standards. The de novo reconstruction may output reconstructed, fragmentary, peptide sequences (contigs) with a probability assigned to each amino acid as well as the assembled peptide sequence. In some instances, a k-mer or De Brujin approach may be used for peptide sequence reconstruction. For example, reads arising from each polymerizable molecule may be broken down into shorter k-mer sequences. The k-mer sequences from the pool of reads may be assembled into longer contig sequences. A De Brujin graph may be generated, e.g., to represent splice variants, post-translational modifications, or other proteoforms. The isoforms may be assembled and the expression level may be determined using a Bayesian approach. The assembled isoforms of proteins may be subjected to evaluation and error correction, e.g., by comparison with standard proteins that are spiked in samples, and assessing for missing segments of sequences, incorrect or redundant assembly, uniform coverage, etc.

Alternatively or in addition to, the binding agent may comprise a detectable label or moiety. For example, the binding agent may comprise a fluorophore, radioisotope, mass tag, chromogenic enzyme (e.g., horse radish peroxidase), etc., which may be detectable using the appropriate imaging technique. Different binding agents (e.g., binding agents that recognize different monomers or amino acids) may be labeled with distinct labels, e.g., different fluorophores, which can be used to identify the presence of the monomer or amino acid. In some examples, fluorophore-labelled binding agents can be detected using single molecule imaging (e.g., total internal reflection, confocal, wide-field, or super resolution microscopy (e.g., PALM, STORM, STED), fluorescence lifetime imaging microscopy (FLIM)).

FIG. 2C schematically shows another example workflow of sequencing a polymeric analyte using an imaging approach. A plurality of polymeric analytes (e.g., peptides) may be coupled to a substrate 201, along with a plurality of capture moieties. For illustration purposes, further sequencing workflow operations are shown for a single polymeric analyte 203; however, it will be appreciated that the workflow operations of FIGS. 2A-2C may be performed on all or a subset of the polymeric analytes in parallel. In process 210, a linker 209 is provided. The linker 209 is capable of coupling to (i) a monomer of the polymeric unit (e.g., a terminal amino acid, such as the N-terminal amino acid) and (ii) a capture moiety 205, which may be used to locally tether the monomer adjacent to the polymeric analyte. In an example, the linker 209 may comprise an amino acid-reactive group such as phenyl isothiocyanate (PITC), which enables the linker to couple to an N-terminal amino acid. The linker may additionally comprise a substrate-tethering moiety that can couple to the capture moiety. For example, the substate-tethering moiety may comprise a click chemistry moiety that can couple to a click chemistry capture moiety, e.g., through an azide-alkyne or azide-cycloalkyne reaction. In other examples, the substrate-tethering moiety may comprise a nucleic acid molecule that can couple, e.g., via hybridization, ligation, or both, to a nucleic acid capture moiety (e.g., as shown in FIG. 2B). In process 212, the substrate-tethering moiety of the linker may couple to the capture moiety. In process 213, the monomer may be cleaved from the polymeric analyte. In some embodiments, cleavage of the monomer may be mediated by a stimulus, e.g., chemical reaction or pH change (such as addition of acid). Subsequent to cleavage, a binding agent 215 may be provided. The binding agent, e.g., an antibody or antibody fragment, may be specific to a particular monomer of a plurality of monomers, e.g., specific to a particular amino acid type or derivative thereof. The binding agent may comprise a detectable label (e.g., fluorophore, radioisotope, mass tag, etc.). The detectable label may be detected (e.g., using microscopy or imaging). To determine the identity of the cleaved terminal monomer (e.g., amino acid). Subsequently, the linker may be removed or cleaved, and the process may be repeated or reiterated to sequence the remaining monomers of the polymeric analyte.

Iteration: One or more of the operations or processes described herein may be iterated or repeated. Iteration of the operations may allow for sequential processing, analysis, or identification of the individual monomers of the polymeric analyte, which can allow for reconstruction of the entire polymeric analyte or a portion thereof. For example, referring to FIG. 2B, the operations of the workflow may be conducted to encode the identity (e.g., via the polymerizable molecule 217) of a terminal amino acid (e.g., NTAA) onto the additional capture moiety 207. The operations of workflow 200 may then be repeated to encode the identities of the n-1 terminal amino acid, the n-2 terminal amino acid, the n-3 terminal amino acid, etc., until the entire or portion of the peptide is processed. The encoding may occur on the same (additional) polymerizable molecule 207, e.g., to generate a stacked polymerizable molecule comprising multiple polymerizable molecules from multiple binding agents, or the encoding may occur on additional polymerizable molecules (not shown) present on the substrate. In the former situation, in some instances, the polymerizable molecule of the second (or third, fourth, fifth, . . . nth) cycle may be configured to only couple to the first (or second, third, fourth, . . . n-1th) polymerizable molecule. For example, the first cycle binding agent polymerizable molecule may comprise a unique binding sequence that is absent on the additional polymerizable (or capture) molecules of the substrate, and to which the second cycle binding agent polymerizable molecule can bind. Accordingly, the second cycle binding agent polymerizable molecule can only bind to the first cycle binding agent polymerizable molecule and not to any of the additional polymerizable (or capture) molecules of the substrate. In the event that no binding occurs (a “null” event), a bridging polymerizable molecule may be provided that encodes for a null binding event but comprises the unique binding sequence, such that subsequent rounds may continue, even if a binding agent does not bind the cleaved monomer.

Similarly, any of the operations depicted in FIGS. 2A-2C may be iterated to sequentially analyze all or a subset of the monomers of the polymeric analyte. For instance, referring to FIG. 2B, the operations of the workflow may be conducted to encode the identity (e.g., via the polymerizable molecule 217) of a terminal monomer (e.g., NTAA) onto an additional capture moiety (not shown) or onto the barcoded capture moiety. The operations may be repeated to encode the identities of the n-1 terminal amino acid, the n-2 terminal amino acid, the n-3 terminal amino acid, etc. until the entire or portion of the peptide is processed. The encoding may occur on the same capture moiety 205, e.g., to generate a stacked polymerizable molecule comprising multiple barcode polymerizable molecules 217, or the encoding may occur on additional capture moieties (not shown). In the former situation, in some instances, the polymerizable molecule of the second (or third, fourth, fifth, . . . nth) cycle may be configured to only couple to the first (or second, third, fourth, . . . n-1th) polymerizable molecule, as described above.

In instances where one or multiple additional polymerizable molecules are used, the polymerizable molecules 217 (e.g., coupled to the binding agents or provided separately after sorting) may additionally encode temporal information, e.g., the cycle or iteration number, such that the order of the individual monomers may be determined. For example, for a given peptide, the terminal amino acid may be coupled to a capture moiety and cleaved, then contacted with a binding agent comprising a barcode sequence that identifies (i) the identity of the amino acid (e.g., any one of twenty proteinogenic amino acids) and (ii) the cycle number (e.g., cycle 1) (not shown). The information encoded by the barcode sequence may be coupled to an adjacent (additional) polymerizable molecule (not shown) or to the capture moiety 205 or barcoded capture moiety. Following cleavage of the monomer from the capture moiety (e.g., as shown in process 221 of FIG. 2B), the workflow may be repeated for the n-1 terminal amino acid, which may again be coupled to a capture moiety, cleaved, and contacted with a binding agent, which may comprise an additional barcode sequence that identifies (i) the identity of the amino acid (e.g., any one of twenty proteinogenic amino acids) and (ii) the cycle number (e.g., cycle 2). The information encoded by the additional barcode sequence may be transferred to the same capture moiety 205 or barcoded capture moiety, or to an additional polymerizable molecule (not shown). In the former situation, the polymerizable molecule may then comprise information on the (i) the identity of the terminal amino acid, (ii) the cycle number of the terminal amino acid (cycle 1), (iii) the identity of the n-1 terminal amino acid, and (iv) the cycle number of the n-1 terminal amino acid (cycle 2), and so forth. Alternatively, or in addition to, the temporal information may be provided on another molecule, such as the capture moiety 205, the linking nucleic acid molecule 211, additional polymerizable molecule 207, etc.

Alternatively, or in addition to, the binding agent may comprise a sorting tag, and a barcode sequence may be provided subsequent to sorting. For example, the binding agents may be used to sort different cleaved amino acid-linker complexes (as shown in process 219), and a polymerizable molecule 217 comprising the identity of the amino acid type may be provided for each sorted, cleaved amino acid-linker complex. The polymerizable molecule 217 may also comprise temporal information, e.g., the round or cycle in which it is provided.

In some instances, temporal information may be provided separately. For example, prior to, during, or subsequent to coupling of a polymerizable molecule 217 comprising barcode information to the capture moiety (FIG. 2B), a temporal barcode may be provided that can couple to the polymerizable molecule 217 of the binding agent or to the capture moiety 205, or a combination thereof. The temporal barcode may comprise any useful agent, including a nucleic acid molecule, a peptide, a lipid, a carbohydrate, an enzyme (e.g., a chromogenic or fluorogenic enzyme) or a ribozyme or DNAzyme, a fluorophore, a dye, an intercalating agent, a dideoxynucleotide, a fluorescent nucleic acid molecule or nucleotide, a radioisotope, a mass tag, or other detectable label that can indicate the time or cycle (or iteration) number in which it is provided. In some instances, the temporal barcode comprises a cycle-specific nucleic acid barcode molecule, which can couple to the polymerizable molecule 217 (comprising the identity of the monomer) or to a terminal polymerizable molecule of a stacked polymerizable molecule comprising polymerizable molecules from multiple rounds or iterations. The temporal barcode may comprise any additional useful functional sequences, e.g., primer sites, sequencing sites, restriction sites, abasic or cleavable sites, etc. In some instances, the temporal barcode may comprise an amplification site that allows for bridge amplification of the temporal barcode and optionally, the coupled polymerizable molecules, to other capture or polymerizable molecules.

Polymeric Analytes: The polymeric analyte described herein may be a biomolecule, macromolecule, or synthetic molecule. The polymeric analyte may be a biomolecule or other biological molecule that comprises one or more monomers. Non-limiting examples of polymeric biomolecules include nucleic acid molecules (e.g., DNA molecule, RNA molecule, DNA: RNA hybrids, aptamers), peptides and proteins, polysaccharides, lipid polymers (e.g., diglycerides, triglycerides and other fatty acids). The polymeric analyte may be a synthetic molecule, e.g., a peptoid or synthetic polymer, or a peptidomimetic (e.g., a peptoid, a beta-peptide, a D-peptide peptidomimetic). Non-limiting examples of synthetic polymers include acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride), or a combination thereof. The polymeric analytes may comprise a single polymer type (e.g., a homopolymer) or more than one polymer type (e.g., a copolymer) and may comprise random or arranged monomers. The polymeric analytes may be a block polymer, alternating copolymer, periodic copolymer, statistical copolymer, stereoblock copolymer, gradient copolymer, branched copolymer, graft copolymer, etc.

The polymeric analytes may be any size or comprise a range of sizes. The polymeric analyte may be about 1 nanometer (nm), about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (μm), about 10 μm, about 100 μm, about 1 millimeter mm in size or greater. A plurality of polymeric analytes may comprise polymeric analytes of similar size or within a range of sizes, e.g., between about 10 nm to about 100 nm, between about 50 nm to about 1 μm. Similarly, the polymeric analytes may have any molecular weight or range of molecular weights. The polymeric analytes may be about 10 daltons (Da), 100 Da, 500 Da, 1 kilodalton (kDa), 10 kDa, 100 kDa, 1,000 kDa, 10,000 kDa, 100,000 kDa, or greater. The polymeric analytes may comprise polymeric analytes of similar molecular weight or within a range of molecular weights.

The monomers of the polymeric analytes may comprise any size or range of sizes that is less than that of the entire polymeric analyte. A monomer may be about 0.1 nanometer (nm), about 0.5 nm, 1 about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (μm), about 10 μm, about 100 μm, about 1 millimeter mm in size or greater. The monomers may have any molecular weight or range of molecular weights. The monomers may be about 1 dalton (Da), 10 Da, 100 Da, 500 Da, 1 kilodalton (kDa), 10 kDa, 100 kDa, 1,000 kDa, 10,000 kDa, 100,000 kDa, or greater. The monomers or polymeric analytes may range in size of molecular weight; for example, a polymeric analyte may comprise a peptide comprising amino acid monomers, which may vary in molecular weight from 75 Da (glycine) to 204 Da (tryptophan).

The polymeric analytes may comprise any number of monomers. The polymeric analytes may comprise about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000 or more monomers. The polymeric analytes may comprise at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 500, at least about 1,000, at least about 5,000, at least about 10,000, at least about 50,000 at least about 100,000 or greater monomers. Alternatively, the polymeric analytes may comprise at most about 100,000, at most about 50,000, at most about 10,000, at most about 5,000, at most about 1,000, at most about 500, at most about 100, at most about 50, at most about 10, at most about 5, or fewer monomers. The polymeric analytes may comprise a range of monomers; for example, a polymeric analyte may comprise about 5 monomers whereas another polymeric analyte may comprise about 500 monomers.

In some instances, the polymeric analyte comprises a peptide comprising amino acid monomers. The peptide may be naturally occurring or synthetic. The peptide may comprise any number of amino acids. The amino acids may be one of 20 proteinogenic amino acids and may comprise any number of post-translational modifications. The peptides or any of the constituent amino acids may be processed, e.g., contacted with protecting groups, alkylated, beta-elimination of phosphate groups, etc., as is described elsewhere herein. In some instances, the peptides are derived from larger peptides or proteins and are fragmented.

Substrates: One or more operations described herein may be performed using a substrate. For example, one or more molecules described herein (e.g., polymeric analyte such as a peptide, capture moiety, polymerizable molecule) may be coupled to a substrate. In some instances, the polymeric analyte, capture moiety, and one or more polymerizable molecules (e.g., the first or second polymerizable molecule), or a combination thereof may be provided coupled to one or more substrates. In one example, the polymeric analyte and a capture moiety are coupled to a substrate. The substrate may comprise one or more anchor molecules that may couple to the polymeric analyte or the capture moiety. In some instances, more than one substrate may be used. In such cases, the substrates may comprise the same material or different material.

The substrate may be made from any suitable material, e.g., glass, silicon, gel (e.g., a hydrogel including reversible hydrogels), polymer, etc., as is described elsewhere herein. In some instances, the substrate may be a bead or a gel bead (e.g., polyacrylamide, agarose, or TentaGel® bead). The substrate may comprise a flow cell, a microfluidic device, or one or more surfaces disposed thereon. The substrate may be functionalized. One or more molecules, e.g., a capture moiety and the polymeric analyte (e.g., a peptide) may be coupled to the substrate via a covalent or non-covalent interaction. The capture moiety and polymeric analyte (e.g., peptide) can be coupled to the substrate using any suitable chemistry, e.g., click chemistry moieties (e.g., alkyne-azide coupling), photoreactive groups (e.g., benzophenone), 1-ethyl-3-(3-dimethylaminopropyl) carbodiimide hydrochloride (EDC) (e.g., to couple amino-oligos or peptides), N-hydroxysulfosuccinimide (NHS), Sulfo-NHS, or NHS-esters (e.g., to couple sulfhydryl oligos), maleimides, hydrazines, hydroxyl amines, thiols, biotin-streptavidin interactions, cystamine, glutaraldehyde, formaldehyde, succinimidyl 4-(N-maleimidomethyl)cyclohexame-1-carboxylate (SMCC), Sulfo-SMCC, 4-(4,6-Dimethoxy-1,3,5-triazin-2-yl)-4-methylmorpholinium chloride (DMTMM), silane (e.g., amino silanes), combinations thereof, etc. In some instances, the substrate may be functionalized to comprise a coupling chemistry to couple the polymeric analyte or the capture moiety. In one non-limiting example, a substrate (e.g., bead or surface) may comprise an alkyne such as dibenzocyclooctyne (DBCO), which may be configured to react to an amine (e.g., DBCO-alcohol, DBCO-Boc, DBCO-NHS), a carboyxl or carbonyl (e.g., DBCO, DBCO-silane), a sulfhydryl, etc. An azide-functionalized nucleic acid or protein may react with DBCO to link the nucleic acid or protein to the DBCO substrate. In other examples, linkers such as bifunctional linkers may be used to attach a molecule to a substrate; such bifunctional linkers may comprise the same reactive moiety on both ends or a different moiety at each end (e.g., heterobifunctional linker). Additional examples of linkers are described elsewhere herein.

In some instances, a molecule (e.g., polymeric analytes such as peptides, capture moieties, polymerizable molecules) may be coupled to the substrate or an anchor molecule of the substrate using an enzymatic approach, e.g., as described elsewhere herein. For example, a chemical linker or moiety such as a click chemistry moiety may be attached to a polymeric analyte (e.g., peptide) using an enzyme. The chemical linker or moiety may be able to react with another chemical linker or moiety (e.g., click chemistry moiety) of a substrate, capture moiety, or polymerizable molecule.

The substrates may be coupled to any useful number of molecules (e.g., polymeric analytes, modified monomers, stacked plurality of modified monomers, capture moieties, polymerizable molecules). For example, the substrate may be coupled to 1, 2, 3, 4, 5, 6, 7, 8, 9, 10, 20, 30, 40, 50, 60, 70, 80, 90, 100, 500, 1000, 5000, 10000, 100000, 500000, 1000000, 10000000 or more molecules. In some instances, a substrate may comprise a plurality of polymeric analytes (e.g., peptides) and a plurality of anchor molecules, which may be provided at any useful ratio or density. For example, the ratio of polymeric analytes, modified monomers, or stacked plurality of modified monomers to anchor molecules may be about 1:1, 1:5, 1:10, 1:20, 1:100, 1:1000, 1:10,000, 1:100,000, 1:1,000,000 or lower. In some instances, the ratio of polymeric analytes to anchor molecules may be at most about 1:1, at most about 1:5, at most about 1:10, at most about 1:20, at most about 1:100, at most about 1:1000, at most about 1:10,000, at most about 1:100,000, at most about 1:1,000,000 or lower.

Similarly, the molecules (e.g., polymeric analytes, modified monomers, stacked plurality of modified monomers, capture moieties, anchor molecules, or polymerizable molecules) may be coupled to the substrate at any useful density, for example about 1 molecule/square micron (μm²), about 10 molecules/μm², about 100 molecules/μm², about 1,000 molecules/μm², about 10,000 molecules/μm², about 100,000 molecules/μm², about 1,000,000 molecules/μm², about 10,000,000 molecules/μm², about 100,000,000 molecules/μm², about 1,000,000,000 molecules/μm², about 10,000,000,000 molecules/μm², about 100,000,000,000 molecules/μm², or greater. The polymeric analytes, capture moieties, anchor molecules, and/or polymerizable molecules may be coupled to the substrate at a range of densities, e.g., from about 100 to about 10,000 molecules/μm², or from about 10 to about 1,000 molecules/μm². The density of the polymeric analytes, capture moieties, anchor molecules, and/or polymerizable molecules may be the same or different. For example, the density of the polymerizable molecules may be 1-fold, 2-fold, 3-fold, 4-fold, 5-fold, 6-fold, 7-fold, 8-fold, 9-fold, 10-fold, 100-fold, 1000-fold, 10,000-fold, 100,000-fold, 1,000,000-fold or greater-fold lower than that of the polymeric analyte.

In some instances, the molecules coupled to the substrate may be spaced apart at a designated or controlled distance. For example, the average spacing or distance between the anchor molecules, the capture moieties, or the processed polymeric analyte, e.g., the stacked plurality of modified monomers, may be spaced at a pitch of about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 μm, about 5 μm, about 10 μm or greater. In some instances, the spacing between or pitch of the molecules (e.g., anchor molecules, capture moieties, polymeric analyte, or processed polymeric analyte such as the stacked plurality of modified monomers) may be at most about 10 μm, at most about 5 μm, at most about 1 μm, at most about 500 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 5 nm, or less. Similarly, the spacing or distance between a polymeric analyte and a polymerizable molecule or capture moiety may be about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 μm or greater. In some instances, the average spacing between the capture moiety and the polymeric analyte coupled to the substrate may be at most about 1 μm, at most about 500 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 5 nm, or less. A range of average distances between the polymerizable molecules from one another or from the polymeric analytes may be used, e.g., from about 1 nm to about 40 nm, from about 2 nm to about 10 nm, etc. In some instances, the substrate may be coupled to only a single molecule. In some instances, the molecules may be spaced or distributed unevenly across the substrate (e.g., with varying distances between the molecules).

The concentration or density of the molecules attached to the substrate may be modulated using one or more suitable approaches, including patterning or random deposition approaches. Examples of methods to control the concentration or density of the molecules attached to the substrate include limited dilution, addition of chaotropes (e.g., guanidine, formamide, urea), using metal organic compounds, etc. The molecules may be attached to the substrate in a patterned fashion, e.g., using self-assembling monolayers, photopatterning, lithography, etching, or a combination thereof, or the molecules may be randomly arranged.

The substrate may comprise any useful size or dimension (e.g., length, width, height, diameter, radius), surface area, volume, or ratio or combination thereof. The substrate may comprise a bead or particle that may comprise a diameter of about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1 millimeter (mm) or greater. The substrate may comprise a surface area of about 1 square nanometer (nm²), about 10 nm², about 100 nm², about 1,000 nm², about 10,000 nm², about 100,000 nm², about 1 μm², about 10 μm², about 100 μm², about 1,000 μm², about 10,000 μm², about 100,000 μm², about 1 mm², about 10 mm², about 100 mm², about 1,000 mm², about 10,000 mm², about 100,000 mm², about 1,000,000 mm² or greater.

The molecules may be coupled to the substrate in an ordered, semi-ordered, or random arrangement. In ordered arrangements, the molecules may be patterned using any conventional approach such as lithography (e.g., soft lithography, photolithography), etching (e.g., ion etching, photo etching), or other patterning approach. In some instances, a linker (e.g., bifunctional linker) may be used to facilitate the coupling of the molecules (e.g., polymeric analytes, polymerizable molecules, capture moieties) to the substrate; such linkers may be patterned using any useful technique such as self-assembling monolayers, photopatterning, lithography, etching. In some instances, the molecules may be coupled to the substrate in a random arrangement. For example, the molecules may be provided at a stoichiometric ratio or controlled concentration to couple the molecules at any useful ratio or density. In some instances, the substrate may comprise topographical or patterned features which may facilitate attachment of linkers to the patterned features.

In some instances, the methods provided herein may comprise using a plurality of substrates. For instance, the preparation of the modified monomers or stacked plurality of modified monomers may be performed using a substrate. The modified monomers or stacked plurality of modified monomers may then be removed from the substrate and contacted with an additional substrate for coupling and detection. In one such example, a modified monomer or stacked plurality of modified monomers may be contacted with a flow cell comprising one or more attachment or anchor molecules (e.g., anchor nucleic acid molecule). The modified monomer or stacked plurality of modified monomers may be coupled to the flow cell via one of the anchor molecules, linearized (e.g., using flow or electrophoretic force), and attached at another point to another anchor molecule. The linearized molecule may then be detected, e.g., using fluorescently labeled binders.

Polymerizable Molecules: The polymerizable molecules described herein may be any useful type of polymerizable molecule. The polymerizable molecules may by naturally occurring, such as biological polymers (e.g., nucleic acid molecules, peptides, polysaccharides, fatty acids), or other naturally occurring polymers, e.g., rubber, cellulose, starches, polyhydroxyalkanoates, chitosan, dextran, structural proteins (e.g., collagen, hyaluronic acid, glycosaminoglycans), agarose, carrageenan, isphagula, acacia, agar, gelatin, shellac, xanthan gum, guar gum, alginate, etc. The polymerizable molecules may be synthetic, e.g., acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and combinations thereof. The polymerizable molecules may comprise one or more reactive moieties (e.g., radical groups) to initiate polymerization or may be polymerized via contacting of an initiating agent (e.g., ammonium persulfate, peroxide, or other radicalizing agent). The polymerizable molecules may be polymerizable via contacting of an enzyme (e.g., polymerizing enzyme such as polymerases), ribozyme or DNAzyme. Alternatively or in addition to, the polymerizable molecules may be polymerizable via self-assembly. The polymerizable molecules may comprise a single polymer type (e.g., a homopolymer) or more than one polymer type (e.g., a copolymer) and may comprise random or arranged monomers. The polymerizable molecules may be a block polymer, alternating copolymer, periodic copolymer, statistical copolymer, stereoblock copolymer, gradient copolymer, branched copolymer, graft copolymer, etc.

The same or different types of polymerizable molecules may be used in the methods described herein. For example, a first polymerizable molecule comprised by or coupled to the binding agent may be a nucleic acid molecule, and a second polymerizable molecule may be a peptide. In another example, both the first polymerizable molecule and the second polymerizable molecule are nucleic acid molecules. In such an example, the first polymerizable molecule may be coupled to the second polymerizable molecule via ligation or hybridization. For instance, the first polymerizable molecule may comprise a first nucleic acid sequence and the second polymerizable molecule may comprise a second nucleic acid sequence. The first nucleic acid sequence may be complementary or partially complementary to the second nucleic acid sequence, and the coupling may comprise hybridizing the first nucleic acid sequence or portion thereof to the second nucleic acid sequence or portion thereof. Alternatively, the first nucleic acid sequence and the nucleic acid sequence may be complementary to two sequences of a splint or bridge oligonucleotide, and coupling may be mediated via hybridization to the splint oligo. The first nucleic acid sequence may be ligated to the second nucleic acid sequence, either chemically (e.g., via click chemistry approaches in which the first polymerizable molecule and the second polymerizable molecule comprise one member of a click chemistry pair) or enzymatically (e.g., using a ligase). In another example, both the first polymerizable molecule and the second polymerizable molecule may be peptides, and the peptides may be coupled to one another, e.g., via enzymatic or chemical ligation.

The polymerizable molecules may comprise functional portions. For example, the polymerizable molecules may comprise a nucleic acid molecule comprising a functional sequence, such as a primer sequence (e.g., universal priming site), a sequencing sequence, a read sequence, a spacer, a unique molecular identifier (UMI), a barcode sequence, a cleavage sequence (e.g., a restriction site, a Cas-binding sequence, an abasic site), a transposition sequence (e.g., a mosaic end sequence), or a combination thereof. In some embodiments, the polymerizable molecule may comprise one or more repeat sequences (e.g., a repeat of an N-mer, where N is an integer greater than 1) or a homopolymer sequence (e.g., a sequence of all A, all T, all C, all G). Such repeat or homopolymer sequences may be useful in, for example, further distinguishing the identity of an amino acid type comprised by a stacked plurality of modified amino acids during nanopore sequence (e.g., to establish a current “background” signal that is then punctuated by amino acids adjacent to or entering the constriction zone).

The polymerizable molecules may be any useful size. The polymerizable molecules may be about 1 angstrom, about 2 angstrom, about 3 angstrom, about 4 angstrom, about 5 angstrom, about 6 angstrom, about 7 angstrom, about 8 angstrom, about 9 angstrom, about 10 angstrom, about 20 angstrom, about 30 angstrom, about 40 angstrom, about 50 angstrom, about 60 angstrom, about 70 angstrom, about 80 angstrom, about 90 angstrom, about 100 angstrom, about 200 angstrom, about 300 angstrom, about 400 angstrom, bout 500 angstrom, about 600 angstrom, about 700 angstrom, about 800 angstrom, about 900 angstrom, about 1000 angstrom, about 10,000 angstrom, about 100,000 angstrom or greater in size, length, or another dimension. In some instances, the polymerizable molecule (e.g., the first polymerizable molecule or the second polymerizable molecule) comprises a nucleic acid molecule comprising one or more nucleotide bases. The polymerizable molecule may comprise any useful number of nucleotide bases, e.g., about 1 base, about 2 bases, about 3 bases, about 4 bases, about 5 bases, about 6 bases, about 7 bases, about 8 bases, about 9 bases, about 10 bases, about 20 bases, about 30 bases, about 40 bases, about 50 bases, about 60 bases, about 70 bases, about 80 bases, about 90 bases, about 100 bases, about 200 bases, about 300 bases, about 400 bases, about 500 bases, about 600 bases, about 700 bases, about 800 bases, about 900 bases, about 1000 bases, or a greater number of bases.

The polymerizable molecules may comprise a nucleic acid molecule. The nucleic acid molecule can be single stranded, double stranded, or partially double-stranded. The nucleic acid molecule may comprise a modified nucleotide or non-canonical base. For instance, the polymerizable molecules may comprise a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some instances, a polymerizable molecule may comprise a hexitol nucleic acid (HNA) or a cyclohexyl nucleic acid (CeNA), which may be useful in rendering the polymerizable molecule more resistant to acid degradation (e.g., as used in conventional Edman degradation). Alternatively or in addition to, a polymerizable molecule may comprise naturally occurring bases that are more resistant to acid degradation, e.g., be composed of primarily thymine or cytosine. For example, a nucleic acid molecule may comprise at least 50%, at least 60%, at least 70%, at least 80%, at least 90%, or 100% thymines or cytosines, which can render the nucleic acid molecule more acid resistant as compared to a nucleic acid molecule comprising adenines or guanines.

Linkers: One or more operations of the methods provided herein may be mediated using a linker. In some instances, the coupling of the monomer to the capture moiety to generate a monomer-capture moiety complex is mediated using a linker. The coupling of the linker to the monomer or capture moiety may be covalent or noncovalent. In an example, a linker may comprise a first reactive group that is able to couple to a monomer of the polymeric analyte (e.g., an amino acid of a peptide) and optionally, cleave the amino acid from a peptide. For example, the first reactive group may be an amino-acid reactive group, e.g., an isothiocyanate (ITC) such as phenyl isothiocyanate (PITC), 3-pyridyl isothiocyanate (PYITC), 2-piperidinoethyl isothiocyanate (PEITC), 3-(4-morpholino) propyl isothiocyanate (MPITC), 3-(diethylamino) propyl isothiocyanate (DEPTIC) or naphthylisothiocyanate (NITC), fluorescein isothiocyanate (FITC), ammonium thiocyanate, potassium thiocyanate, trimethylsilyl isothiocyanate (TMS-ITC), phenyl phosphoroisothiocyanatidate, acetyl isothiocyanate (AITC), adamantyl isocyanate (ADIC), or an aldehyde group, e.g., ortho-phthalaldehyde (OPA), 2,3-naphthalenedicarboxyaldehyde (NDA), 2-pyridinecarboxyaldehyde, which can react with an N-terminal amino acid (NTAA). The linker may additionally comprise a second reactive group that is capable of coupling, either directly or indirectly, to the capture moiety. In an example of direct coupling, the capture moiety may comprise a click chemistry moiety (e.g., alkyne), and the second reactive group of the linker may comprise an additional click chemistry moiety (e.g., azide) that can react with the click chemistry moiety of the capture moiety. Alternatively, the linker may be coupled indirectly to the capture moiety, e.g., via noncovalent interaction or via an intermediate linking molecule. In some instances, the intermediate linking molecule may comprise an additional polymerizable molecule (e.g., a polymer or nucleic acid molecule) that can couple the linker to the capture moiety. In one such example, the additional polymerizable molecule may comprise (i) a third reactive group that is capable of coupling to the second reactive group (e.g., via alkyne-azide click chemistry) of the linker and (ii) a moiety that can couple to the capture moiety (e.g., another orthogonal click chemistry reaction, avidin-biotin interaction, nucleic acid coupling or hybridization). In some instances, the third polymerizable molecule comprises a nucleic acid molecule that comprises (i) a click chemistry moiety (e.g., alkyne) that can conjugate to the first reactive group (e.g., azide) of the linker and (ii) a nucleic acid sequence that can couple to the capture moiety, e.g., via ligation, splint ligation, or hybridization. In some instances, the linker comprises a linking nucleic acid molecule that comprises a self-splinting moiety. In some instances, the linker may be incorporated into or be pre-coupled to the linking nucleic acid molecule.

When applicable, the click chemistry moieties of the linker and capture moiety or intermediate linking molecule may comprise any suitable bioorthogonal moieties, as described elsewhere herein, e.g., alkenes, alkynes, azides, epoxides, amines, thiols, nitrones, isonitriles, isocyanides, aziridines, activated esters, and tetrazines, and combinations, variations, or derivatives thereof. The linker may be subjected to conditions sufficient to react the first click chemistry moiety to the second click chemistry moiety, e.g., provision of metal catalysts, appropriate solvents, pH, temperature, ionic concentration, or light/energy for any useful duration of time.

The first reactive group of the linker may be an amino acid-reactive moiety. The amino acid-reactive moiety of the linker may be any useful moiety that enables the reactive moiety to conjugate to and optionally cleave an amino acid. In some examples, the first reactive moiety can react with a terminal amino acid (e.g., NTAA or CTAA). In such examples, the first reactive moiety may comprise any primary amine or carboxylic group reactive group, including but not limited to isocyanates, acyl azides, NHS esters, sulfonyl chlorides, aldehydes, glyoxals, epoxides, oxiranes, carbonates, aryl halides, imidoesters, carbodiimides, anhydrides, phenyl esters, isothiocyanates (e.g., phenyl isothiocyanate, sodium isothiocyanate, ammonium isothiocyanates (e.g., tetrabutylammonium isothiocyanate, tetrabutylammonium isothiocyanate), diphenylphosphoryl isothiocyanate), acetyl chloride, cyanogen bromide, carboxypeptidases, azide, alkyne, DBCO, maleimide, succinimide, thiol-thiol disulfide bonds, tetrazine, TCO, vinyl, methylcyclopropene, acryloyl, allyl, among others. Additional examples of amino acid reactive groups and linkers are provided in U.S. Pat. No. 11,499,979; International Pat. Pub. No. WO2024/107755; International Pat. Pub. No. WO2024/159162; U.S. patent application Ser. No. 18/953,510; International Pat. App. No. PCT/US2025/039826, filed Jul. 30, 2025; International Pat. Pub. No. WO2025/166050A1; and International Pat. App. No. PCT/US2026/013143, filed Jan. 29, 2026, each of which is incorporated by reference herein in its entirety.

The linker may comprise any additional useful moieties. For example, the linker may comprise a releasable or cleavable moiety, which may facilitate removal of the monomer from the polymeric analyte, or portion thereof, or from the substrate. Such a releasable or cleavable moiety may comprise, for example, a disulfide bond, which may be releasable by contacting with a reducing agent (e.g., DTT, TCEP). In some examples, the linker may couple to the third polymerizable molecule via the releasable or cleavable moiety, alternatively or in addition to the coupling via click chemistry moieties. As such, the coupling between the polymerizable molecule and the linker may be reversible. The linker may additionally or alternatively comprise any number of spacing moieties, e.g., polymers (e.g., PEG, PVA, polyacrylamide), aminohexanoic acid, nucleic acids, alkyl chains, etc. Such spacing moieties may increase the distance between any other moieties of the linker, e.g., the amino acid-reactive group and the polymerizable molecule-reactive group. The linker may comprise or be coupled to a detectable moiety, e.g., a fluorophore, radioisotope, mass tag, nucleic acid molecule (which can also act as a releasable or cleavable moiety), or other detectable moiety. In some examples, the linker comprises a fluorophore, which can enable localization visualization of the linker using single-molecule imaging or fluorescence lifetime imaging. In another example, the monomer may be labeled with a first fluorophore and the linker may comprise a second fluorophore to enable localization visualization of the linker and the monomer (e.g., using two-channel imaging or FRET).

Use of a linker comprising two reactive groups may allow for coupling of the linker to (i) the monomer of the polymeric analyte and (ii) the intermediate linking molecule (e.g., third polymerizable molecule) or (iii) the capture moiety. In some instances, when an intermediate linking molecule is used, the linker may be pre-coupled to the intermediate linking molecule. For example, a precursor linker may comprise a monomer-binding group (e.g., PITC) and a click chemistry moiety (e.g., azide), which may be reacted with a polymerizable molecule (e.g., oligonucleotide) comprising complementary click chemistry moiety (e.g., alkyne) to generate a linker that is capable of coupling to the monomer and the capture moiety (e.g., another oligonucleotide). In some instances, the linker may be provided pre-coupled to the intermediate linking molecule.

In some instances where the polymeric analyte comprises a peptide that comprises amino acid monomers, the coupling of the linker to an amino acid (e.g., NTAA or CTAA) changes the chemical structure of the amino acid. For example, if using a linker comprising an isothiocyanate moiety, the amino acid may be derivatized to a thiocarbamyl group (e.g., under mildly alkaline conditions) during or subsequent to contact with the isothiocyanate moiety. One or more further derivatizations may be performed. For instance, the amino acid or amino acid derivative (e.g., thiocarbamyl-derivatized amino acid) may be further derivatized to a thiazolone group (e.g., under acid conditions), a thiohydantoin group, or other chemical moiety. Similarly, a thiazolone group or thiohydantoin group may be further derivatized to a thiocarbamyl group.

Capture Moieties: A capture moiety may couple to the amino acid, the linker, or the polymerizable molecule. The coupling of the amino acid, the linker, or the polymerizable molecule to the capture moiety may comprise a covalent interaction or a noncovalent interaction. The coupling may occur by interaction of binding pairs, e.g., biotin and avidin (or streptavidin), antigen or epitope and antibody or antibody fragment, cyclodextrins and small hydrophobic molecules (e.g., alkanes, benzene, polycyclics), cucurbiturils and adamantaneammonium or trimethylammoniomethyl ferrocene, cyclophane (e.g., calixarenes, cavitands, pillararenes, tetralactams), etc. In some embodiments, the coupling of the amino acid, the linker, or the polymerizable molecule to the capture moiety occurs through coupling of nucleic acid molecules (e.g., hybridization to one another or to a splint molecule or a nucleic acid extension).

In some instances, the capture moiety comprises an additional polymerizable molecule (e.g., a nucleic acid molecule or peptide). In one such example, both the polymerizable molecule of the modified amino acid and the capture moiety may comprise nucleic acid molecules. The nucleic acid molecules may be coupled to one another, e.g., via complementary base pairing directly or via a splint molecule and optional ligation (e.g., enzymatic or chemical ligation). Alternatively, or in addition to, the nucleic acid molecules may be coupled via a nucleic acid extension or amplification reaction. In some instances, the capture moiety and the polymerizable molecule comprise click chemistry moieties or reactive moieties which can allow for chemical ligation of the capture moiety to the polymerizable molecule. Non-exhaustive examples of chemical attachment of oligonucleotides can be found in M. Greenberg. Current Protocols in Nucleic Acid Chemistry. (2000). 1.4.1-4.5.19, which is incorporated by reference in its entirety. In another example, both the polymerizable molecule of the modified amino acid and the capture moiety may comprise peptides. The peptides may be coupled to one another, e.g., via enzymatic or chemical ligation.

The nucleic acid molecule of the capture moiety or the polymerizable molecule, or, if applicable, a splint molecule, can comprise any naturally occurring, non-naturally occurring or engineered nucleotide base. For example, the nucleic acid molecule may comprise a pseudo-complementary base, a bridged nucleic acid, a xenonucleic acid, a locked nucleic acid, a peptide nucleic acid (PNA), a gamma-PNA, a morpholino, etc., as is described elsewhere herein. The capture moiety may comprise one or more functional sequences, including, but not limited to a priming sequence, sequencing sequence (e.g., P5 or P7 sequence), sequencing read sequence (e.g., R1 or R2 sequence), a mosaic end sequence, a transposase recognition sequence, a cleavage site (e.g., restriction site), a UMI, a blocking group, a spacer sequence, a barcode sequence, or other functional sequence. In some instances, the capture moiety comprises a cleavable or releasable moiety, e.g., a restriction enzyme recognition site, an abasic site, a uracil which can be cleaved using USER® or uracil DNA glycosylase, a disulfide bond that can be releasable upon addition of a reducing agent, etc. In some instances, the capture moiety comprises a partial restriction site; e.g., the capture moiety may comprise a first partial restriction site and the polymerizable molecule may comprise a second partial restriction site; upon coupling or ligation of the polymerizable molecule to the capture moiety, the two partial restriction sites may generate a complete restriction site, such that the individual molecules (capture moiety and polymerizable molecule) are not cleavable by restriction digest individually but the ligated or coupled product is. In some instances, the capture moiety comprises a barcode sequence that comprises any useful information, e.g., the identity of the peptide that is to be analyzed, temporal information, spatial information, the origin of the peptide (e.g., from a sample, partition, protein) etc.

In some instances, the capture moiety is provided coupled to a substrate. In one example, the substrate comprises one or more identical capture nucleic acid molecules; these identical capture nucleic acid molecules may act as a capture moiety for coupling to a polymerizable molecule of a modified amino acid, e.g., for a modified amino acid comprising a terminal amino acid, an n-1 amino acid, etc. In some instances, commercially available substrates, e.g., beads (e.g., DNA beads or barcoded beads), flow cells, or chips, e.g., Illumina® HiSeq, iSeq, MiniSeq, NextSeq, NovaSeq, etc. may be used as the substrates described herein. In some instances, the capture moieties may comprise additional useful sequences, e.g., primer sequences (e.g., P5 or P7 sequences) or read sequences (e.g., R1 or R2).

Alternatively, or in addition to, the capture moiety may be configured to couple to a substrate. The substrate may comprise one or more anchor molecules to which the capture moiety binds. Non-limiting examples of capture-anchor molecule binding pairs include biotin-streptavidin, nucleic acid coupling, and click chemistry pairs (e.g., azide-alkyne, TCO-tetrazine, etc.).

The capture moiety may be coupled to a substrate using any useful approach. In some instances, the capture moiety comprises a substrate-tethering group or linker or additional functional group. In some examples, the capture moiety comprises a nucleic acid molecule that comprises a substrate-tethering group, e.g., biotin, a click chemistry moiety such as an azide, that can couple to a substrate, e.g., a substrate comprising streptavidin or a complementary click chemistry moiety that can react with that of the substrate-tethering group. Alternatively, or in addition to, the capture moiety may comprise a nucleic acid molecule, which may be coupled to a substrate-tethered nucleic acid molecule (e.g., an anchor nucleic acid molecule). The capture moiety may additionally comprise a binding sequence, to which another nucleic acid molecule (e.g., a polymerizable molecule that is part of or coupled to the modified amino acid). In some instances, the capture moiety comprises a single-stranded oligonucleotide or a single-stranded region in which a complementary oligonucleotide can hybridize. The complementary oligonucleotide may comprise a detectable label (e.g., fluorophore) that allows for detection of the capture moiety.

Alternatively, the capture moiety may not be coupled to a substrate. For instance, the capture moiety may be directly coupled to the polymeric analyte (e.g., peptide) that is to be analyzed or is undergoing intramolecular expansion. In such examples, the capture moiety may additionally comprise a nucleic acid barcode molecule that encodes the identity of the peptide or the originating sample or partition from which the peptide originated. The capture moiety may be coupled to any useful segment of the peptide, e.g., at a terminus (e.g., C-terminus) or at an internal residue. Similarly, the peptide may be coupled to the capture moiety at any useful segment of the capture moiety (e.g., internally or at an end). In some instances, the capture moiety comprises a nucleic acid molecule and the peptide may be coupled to the capture moiety along the backbone, a nucleobase, a sugar or other location of the capture moiety. In some instances, the peptide is coupled to a 5′ or 3′ end of the capture moiety. Alternatively, or in addition to, the capture moiety may be provided in a solution and may not be coupled to the substrate or the peptide. In some instances, the capture moiety is coupled to both the polymeric analyte and to a substrate.

The capture moiety may be coupled to the polymeric analyte using any useful attachment approach, e.g., click chemistry moieties (e.g., alkyne-azide coupling), photoreactive groups (e.g., benzophenone), 1-ethyl-3-(3-dimethylaminopropyl) carbodiimide hydrochloride (EDC) (e.g., to couple amino-oligos or peptides), N-hydroxysulfosuccinimide (NHS), Sulfo-NHS, or NHS-esters (e.g., to couple sulfhydryl oligos), maleimides, hydrazines, hydroxyl amines, thiols, biotin-streptavidin interactions, cystamine, glutaraldehyde, formaldehyde, succinimidyl 4-(N-maleimidomethyl) cyclohexame-1-carboxylate (SMCC), Sulfo-SMCC, 4-(4,6-Dimethoxy-1,3,5-triazin-2-yl)-4-methylmorpholinium chloride (DMTMM), silane (e.g., amino silanes), combinations thereof, etc. In some instances, the peptide or the capture moiety may be functionalized to comprise a coupling chemistry to couple the polymeric analyte to the capture moiety. In one non-limiting example, a polymeric analyte may comprise an alkyne such as dibenzocyclooctyne (DBCO), which may be configured to react to an amine (e.g., DBCO-alcohol, DBCO-Boc, DBCO-NHS), a carboyxl or carbonyl (e.g., DBCO, DBCO-silane), a sulfhydryl, etc. An azide-functionalized capture moiety, e.g., a capture nucleic acid molecule may react with DBCO to link the polymeric analyte and the capture moiety. In other examples, linkers such as bifunctional linkers may be used to attach a polymeric analyte to a capture moiety; such bifunctional linkers may comprise the same reactive moiety on both ends or a different moiety at each end (e.g., heterobifunctional linker). Additional examples of linkers are described elsewhere herein.

In some examples, the capture moiety and the polymeric analyte (e.g., peptide) are coupled using a linker. In an example, terminal amino acid residue attachment can be achieved by reacting the peptide with a linker comprising (i) an amine-reactive group (e.g., isothiocyanates such as PITC, guanidinylating agents, dithioesters, xanthates, NHS esters, etc.) and (ii) a reactive group (e.g., click chemistry group). The linker can be, for example, PITC-conjugated click chemistry moieties. The linker reacts with and “blocks” the primary amines (e.g., modifies lysines), including the N-terminus. Subsequent cleavage of the N-terminal amino acid (e.g., using an Edman reagent, such as acid), can be performed, and one of the remaining modified lysines may be attached to the capture moiety (e.g., using the click chemistry moiety coupled to the amine-reactive group). Optionally, the peptide may be treated with a protease, e.g., LysC, which cleaves peptides such that a remaining peptide has a C-terminal lysine and such that the remaining peptide comprises a primary amine only at the C-terminal lysine residue and the N-terminus. In some instances, the capture moiety may comprise the amine-reactive group, which can then couple to the N-terminal amine or to the amine of a lysine side chain.

Cleaving: In some instances, generating the modified amino acid further comprises cleaving the amino acid or the amino acid-linker complex from the peptide. The cleaving of the amino acid or amino acid-linker complex may be achieved using any suitable mechanism, such as via application of a stimulus. The stimulus can be, for example, a chemical stimulus, a biological stimulus, a thermal stimulus (e.g., application of heat), a photo-stimulus, a physical or mechanical stimulus, or other type of stimulus or a combination of stimuli. In some instances, the stimulus comprises a chemical stimulus, e.g., a change in pH, application of an acid (e.g., trifluoroacetic acid) or base, addition of a lytic agent, initiating agent, radical-generating agent, reducing agent, etc. In some instances, the chemical stimulus comprises application of a Lewis acid (e.g., boron triflate, boron trifluoride etherate, boron trichloride, boron tribromide, boron triiodide, or scandium triflate). In some instances, the stimulus comprises a biological stimulus, e.g., enzyme (e.g., Edmanase, protease, endonuclease) or ribozyme or DNAzyme that can cleave or catalyze cleavage of the amino acid or amino acid-linker complex.

In some examples, the methods provided herein may comprise using a linker comprising an amino acid reactive group (e.g., PITC, a xanthate, a guanidinylating agent, a dithioester or thiocarbamoyl) and coupling the amino acid reactive group of the linker with the amino acid and cleaving the amino acid from the peptide using a stimulus (e.g., change in pH, temperature). In an example, the linker may comprise a PITC moiety that couples to an NTAA under mildly alkaline conditions to generate a phenylthiocarbamoyl (PTC) derivative of the NTAA, and cleavage of the NTAA from the peptide may be achieved using an Edman degradation reaction (e.g., application of an acid such as trifluoroacetic acid or boron triflate, optionally with heat), to generate a thiazolinone (ATZ) derivative or a phenylthiohydantoin (PTH) derivative. As described elsewhere herein, the linker may comprise a moiety or molecule (e.g., polymerizable molecule such as a nucleic acid molecule) that can also couple to the capture moiety such that the amino acid-linker complex may be coupled to the capture moiety, thereby generating an amino acid-linker-capture moiety complex.

Given the harsh reaction conditions of standard Edman degradation, the polymerizable molecules described herein (e.g., nucleic acid molecules, peptides, lipids) etc. may comprise alterations or modifications to render them more resistant to the reaction conditions. For example, nucleic acid molecules may comprise predominantly pyrimidines (e.g., thymines, cytosines, uracils) which are more resistant to acid degradation and heat as compared to purines (e.g., adenine and guanine). For example, a nucleic acid molecule may comprise at least 50%, at least 60%, at least 70%, at least 80%, at least 90%, or 100% thymines or cytosines. Alternatively, or in addition to, canonical nucleotides may be substituted or may comprise acid-resistant nucleotide analogs, e.g., hexitol nucleic acids, peptide nucleic acids, or other nucleotide analog.

Alternative degradation chemistries may also be employed. Milder degradation under basic conditions for N-terminal amino acid removal can include the use of triethylamine acetate in acetonitrile or other solvent such as water, N, N-dimethylformamide (DMF), or a mixture of solvents. Alternatively, degradation may be achieved using a thioacylation approach, the use of milder acid reagents, e.g., trichloroacetic acid (pKa of 0.66) or dichloroacetic acid (pKa of 1.35), or alternative basic reaction conditions, e.g., using acid-base pairs such as N, N-Diisopropylethylamine (DIPEA), pyridine, acetic acid derivatives, etc. In some instances, use of a base (alkaline conditions) may be sufficient for cleavage. For instance, for linkers comprising guanidinylating agents, use of a mild base can be used to cleave amino acid-linker complex.

C-terminal degradation strategies are also provided herein. C-terminal degradation may comprise Edman-like degradation approaches. C-terminal degradation may employ the use of activating reagents that react with the C-terminal carboxyl group of a peptide, and a derivatizing agent (e.g., a thiocyanate to generate a peptide-thiocyanate or peptide-thiohydantoin). Non-limiting examples of activating reagents include acetyl chloride and acetic anhydride. Alternatively, or in addition to, single-step C-terminal derivatization of a peptide to a peptidyl-thiohydantoin may be performed, e.g., using Schlack-Kumpf approach, in which a peptide is reacted with thiocyanic acid (e.g., in acetone) to generate a peptidyl-thiohydantoin. The peptide-thiohydantoin may be cleaved, e.g., using basic conditions, to generate an amino acid thiohydantoin and remaining peptide.

Cleavage of amino acids may also be achieved using enzymatic or enzyme-analog (e.g., ribozyme or DNAzyme) approaches. Example enzymatic cleavage may include the use of Edmanases (e.g., modified cruzain), aminopeptidases (e.g., Pfu aminopeptidase I, PhTET aminopeptidases, P. horikoshii aminopeptidases), metalloenzymatic aminopeptidases, acylpeptide hydrolases, tRNA synthetases, endopeptidases, carboxypeptidases, and the like. The enzymes or ribozymes or DNAzymes may be modified or engineered to recognize a modified amino acid, e.g., an amino acid that has a chemical moiety attached thereto (e.g., PITC, NITC, dansyl chloride, dabsyl chloride, SNFB, DNP, SNP, guanidinyl group, biotin, streptavidin, nucleic acid molecules, lipids, carbohydrates, acetyl groups, acyl groups, guandinylation agents, etc.).

One or more reactions may be accelerated by application of energy or radiation, e.g., electromagnetic radiation. For example, degradation or cleavage of the terminal amino acid of a peptide may be facilitated by applying microwave energy to accelerate the reaction kinetics. For example, hydrolysis of proteins may be facilitated by application of microwave energy, e.g., as described in Margolis et al., 1991, Journal of Automatic Chemistry. Vol 13, No. 3, pp 93-95, which is incorporated by reference herein.

Additional processing of the cleaved amino acids may also be performed. For instance, in some instances, cleavage of the amino acid may result in generation of stereoisomers. The cleaved products may be treated to remove the stereocenter during the cleavage reaction. In some instances, stereoisomers may be enzymatically converted to a single isomer, e.g., using an isomerase.

In some instances, more than one amino acid may be cleaved from the peptide per cleavage event. The cleaving may comprise cleaving 2 amino acids, 3 amino acids, 4 amino acids, 5 amino acids, 6 amino acids, 7 amino acids, 8 amino acids, 9 amino acids, 10 amino acids, or more. For example, the polymeric analyte may comprise a peptide comprising a plurality of amino acids, and single amino acids, di-peptides, tri-peptides, quadri-peptides, or larger may be cleaved in the methods described herein. In some instances, at most about 10 amino acids, at most about 9 amino acids, at most about 8 amino acids, at most about 7 amino acids, at most about 6 amino acids, at most about 5 amino acids, at most about 4 amino acids, at most about 3 amino acids, or fewer amino acids may be cleaved in a given cleavage event. In some instances, cleavage of greater than one amino acid may be mediated using an enzyme (e.g., Edmanase, protease) or ribozyme or DNAzyme that is capable of recognizing or cleaving more than a single amino acid.

Cleavage of the amino acid from the peptide may be conducted using a biological stimulus, such as an enzyme or ribozyme or DNAzyme. The enzyme can be any useful cleaving enzyme, e.g., a protease, such as an Edmanase, cruzain, a cleaving protein (e.g., ClpS, ClpX), Proteinase K, exopeptidase, aminopeptidase, diaminopeptidase, serine protease, cysteine protease, threonine protease, aspartic protease, aspartic protease, glutamic protease, metalloprotease, asparagine peptide lyase, pepsin, trypsin, pancreatin, Lys-C, Lys-N, Arg-C, Glu-C, Asp-N, chymotrypsin, legumain, humicolin, granzyme M, papaya proteinase 4, proalanase, neutrophil elastase, carboxypeptidase (e.g., carboxypeptidase A, carboxypeptidase B, carboxypeptidase Y), SUMO protease, elastase, papain, endoproteinase, proteinase, TrypZean®, bromelain, collagenase, hyaluronase, thermolysin, ficin, keratinase, tryptase, fibroblast activation, enterokinase, chymotrypsinogen, chymase, clostripain, calpain, alpha-lytic protease, proline specific endopeptidase, furin, thrombin, subtilisin, genenase, PCSK9, cathepsin, prolidase, methionine aminopeptidase, cathepsin C, 1-cyclohexen-1-yl-boronic acid pinacol ester, pyroglutamate aminopeptidase, renin, kininogen, kallikrein, DPPIV/CD26, thimet oligopeptidase, prolyl oligopeptidase, leucine aminopeptidase, dipeptidylpeptidase, or other enzyme or protease, or a combination or variation (e.g., engineered mutant or variant) thereof. In some instances, the cleaving enzyme or ribozyme or DNAzyme may be configured or engineered to cleave a terminal amino acid or plurality of amino acids; alternatively, the cleaving enzyme or ribozyme or DNAzyme may be configured or engineered to cleave off-site at a non-terminal location of the peptide, e.g., at an internal amino acid at an n-1, n-2, n-3, n-4, n-5, n-6, n-7, n-8, n-9, n-10, etc. position, where n is the number of amino acids in the peptide.

In the instances of enzymatic cleavage, additional reagents may be provided to catalyze or induce the cleavage. For instance, metalloproteases, aminopeptidases, or exopeptidases may facilitate cleavage of an amino acid or plurality of amino acids in the presence of a catalyst, e.g., metal or metal ion (e.g., cobalt). Accordingly, a catalyst may be provided in order to facilitate the binding of the enzyme to an amino acid or the subsequent cleavage of the amino acid from the peptide. In some examples, cleavage may be mediated by an apo-enzyme, which is inactive in the absence of a metal catalyst of cofactor, and cleavage may be controlled by addition of metal or metal ions.

Other examples of cleaving stimuli include: a photo stimulus (e.g., application of UV, X-rays, gamma rays, or other wavelength of light), mechanical stimulus (e.g., sonication, high pressure, electromagnetic energy), thermal stimulus (e.g., application of heat), or chemical stimulus. In some instances, the peptide may comprise or be altered to comprise a cleavable or labile bond that can be cleaved upon application of the appropriate stimulus, e.g., disulfide bonds (e.g., cleavable upon application of a chemical stimulus such as a reducing agent), ester linkages (e.g., cleavable with a change of pH), a vicinal-diol linkage (e.g., cleavable with sodium periodate), a Diels-Alder linkage (e.g., cleavable upon application of heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNase)).

In some instances, the capture moiety may be cleaved from the peptide or the substrate. The cleaving may occur at any useful or convenient step, e.g., after generation of the modified amino acid or stacked plurality of modified amino acids. In some instances, cleavage of the capture moiety may occur subsequent to the formation of a stacked plurality of modified amino acids, and the cleaved product may be sequenced, e.g., using nanopore sequencing or imaging approaches described elsewhere herein.

Modifications of Monomers: In some instances, one or more monomers of the polymeric analyte may be modified. Modifications may be naturally-occurring (e.g., post translational modifications) or non-naturally occurring, such as by labeling or tagging, e.g., with an amino acid- or amine-reactive agent such as an isothiocyanate (e.g., PITC, NITC), 1-fluoro-2,-4-dinitrobenzene (DNFB), dansyl chloride, dabsyl chloride, 4-sulfonyl-2-nitrobfluorobenzene (SNFB), an acetylating agent, an acylating agent, an alkylating agent, a guanidination agent, a thioacetylation agent, a thioacylation agent, a thiobenzoylation agent, or a derivative or combination thereof. Alternatively, or in addition to, the one or more monomers may be modified to comprise any useful moiety such as an adduct (e.g., a polymer such as PEG, a polymerizable molecule such as a nucleic acid molecule, a nanoparticle or nanotube, a peptide or protein), a lipid, a carbohydrate, a metabolite, a fluorophore, a hapten, a quencher, a tag (e.g., a fluorescent tag, a magnetic tag, a radioactive tag), a barcode, or other moiety. In some instances, a monomer of the polymeric analyte may be modified to facilitate recruitment of an enzyme to recognize or cleave a terminal monomer (e.g., a NTAA or CTAA of a peptide, the 5′ or 3′ nucleotide of a nucleic acid molecule, or the first or last monomer of a polymer) or set of monomers. For example, a terminal amino acid of a peptide analyte may be modified with a saccharide in order to recruit a lectin or lectin-bound protease. In another example, one or more monomers of a polymeric analyte may comprise or be coupled to a nucleic acid molecule having a first sequence that is complementary to a second sequence comprised by an oligo-bound protease. Hybridization of the first sequence to the second sequence may facilitate local recruitment of the protease to the monomer to be cleaved. In yet another example, a peptide analyte may be modified with PITC, which may allow for recruitment and cleavage by an Edmanase. In some examples, modifications to monomers of a polymeric analyte may include epitope tags, which can facilitate binding of a binding agent (e.g., subsequent to cleavage of the monomer from the polymeric analyte). Examples of such epitope tags include fluorophores, nucleic acid molecules, peptides, haptens, polymers, chemical moieties, or other adduct molecule. Additional examples of modifications to polymeric analytes are described elsewhere herein.

The polymeric analyte may comprise one or more modified monomers. The modification of the monomers may be naturally occurring, or synthetic. Synthetic modifications may be performed prior to, during, or subsequent to cleavage of a monomer from the polymeric analyte and may be advantageous in preserving the identity of the monomer. For instance, during standard Edman degradation reactions to cleave a terminal amino acid (monomer) from the peptide, some amino acid residues may be altered or rendered undetectable by the reaction conditions. In an example, the conditions of Edman degradation may cause oxidation of cysteine residues, dehydration or destruction of the phenylthiohydantoin (PTH) forms of serine or threonine, react with and modify lysine residues, or render some post-translational modifications undetectable. As such, it modifying a peptide prior to analysis, e.g., to protect some of the amino acid residues or post-translational modifications may be useful in more accurately identifying each of the amino acid residues. In an example of a modification that can be performed prior to cleavage, a peptide or portion thereof may be alkylated, e.g., to alkylate the cysteine residues (e.g., using 4-vinylpyridine, iodoacetamide, iodoacetate, chloroacetate or aminoethylated, e.g., using 2-bromoethylamine); acetylated, e.g., to react serine or threonine residues form an ester (e.g., using acetyl chloride) or using acetic anhydride; oxidized, e.g., to convert cysteine residues to cysteic acid; reduced (e.g., using a reducing agent such as dithiothreitol, β-mercaptoethanol, or TCEP); subjected to native chemical ligation; contacted with a protecting group, e.g., phosphorylated residues may be protected (e.g., using a β-elimination of a phosphate group, with an optional Michael addition of a thiol group, e.g., as described in Knight, et al. Nat. Biotechnology. 21, 1047-1054 (2003), which is incorporated by reference herein in its entirety), etc. The polymeric analyte or monomer may be modified with a protecting group or moiety, such as a methyl, formyl, ethyl, acetyl, t-butyl, anisyl, benzyl, tifluoroacetyl, N-hydroxysuccinimide, t-butyloxycarbonyl (Boc), benzoyl, 4-methyl benzyl, thioanizyl, thiocresyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl (FMOC), triphenylmethyl, or 2,2,5,7,8-pentamethyl-chroman-6-sulphonyl group. The polymeric analyte or monomer may be treated with a protecting agent, e.g., carboxyethyl methanethiosulfonate (CEMTS), thiazolidine, mercaptophenyl acetic acid, cyanobenzothiazole (e.g., for lipidation of N-terminal cysteines), acetamidomethyl, 2-methylsulfonylethyl-oxycarbonyl, etc. In some instances, the lysine residues may be blocked (e.g., the primary amines of lysine residues may be reacted) using an isothiocyanate (e.g., PITC), and optionally carrying out a single round of Edman degradation to generate a new N-terminal exposed end.

In some instances, a monomer of the polymeric analyte may be modified to facilitate cleavage of the monomer from the polymeric analyte. For example, an amino acid monomer of a peptide polymeric analytic may be modified such that it is recognized by an enzyme, e.g., acetylation of an amino acid, which can facilitate acyl peptide hydrolase cleavage of the acetylated amino acid. Additional or alternative modifications to the monomers, such as those described herein, may also facilitate recognition by or interaction with an engineered cleaving enzyme.

In some instances, a monomer comprising a naturally-occurring modification may be treated to remove or alter the naturally-occurring modification to render the polymeric analyte or monomer more amenable to the processing operations disclosed herein. For example, acetylation, formylation, methylation, and pyrrolidone carboxylic acid post-translational modifications may be removed prior to sequencing. Acetylation modifications may be removed with acyl peptide hydrolase or acid treatment (e.g., using IN HCl). Methylation may be removed using aminopeptidases. Formylation modifications may be removed, for example, using acid treatment (e.g., 0.6M HCl treatment). Pyrrolidone carboxylic acid (PCA) may be removed with pyroglutamate aminopeptidase. Exemplary C-terminal modifications may include amidation and methylation, both of which may be removed using carboxypeptidases.

Binding agents: The binding agent may be contacted with the monomer (e.g., subsequent to cleavage and monomer-capture moiety coupling). The binding agent may be any useful molecule that can couple to the monomer or monomer-capture moiety complex. For example, a binding agent may be or comprise a protein or peptide (e.g., an antibody, antibody fragment, single chain variant fragment (scFv), nanobody, anticalin, tRNA synthetase or tRNA-acyl synthetase, a fibronectin domain), a peptide mimetic, a peptidomimetic (e.g., a peptoid, a beta-peptide, a D-peptide peptidomimetic), a polysaccharide, a nucleic acid molecule (e.g., aptamer), a somamer, a polymer, an inorganic compound, an organic compound, a small molecule, or derivatives (e.g., engineered variants) or combinations thereof. In instances where the polymeric analyte comprises a peptide, the binding agent may be able to bind to a modified amino acid (e.g., an amino acid coupled to a linker) or portion thereof. The binding agent may comprise a recognition site that specifically recognizes an amino acid, modified amino acid (e.g., an amino acid bound to a linker comprising a PITC moiety), or a derivatized (and optionally modified) amino acid. For example, the binding agent may be configured to recognize or have binding specificity to a moiety of a modified amino acid, such as a specific amino acid residue, the residue-linker complex, or derivatized amino acid (e.g., a thiocarbomyl-derivatized residue, a thiazolone-derivatized residue, a thiohydantoin-derivatized residue, etc.), or a portion of a modified amino acid. In some instances, the binding agent may be derived or engineered from a naturally-occurring enzyme or protein, e.g., an aminopeptidase, exopeptidase, metalloprotease, antibody, anticalin, N-recognin protein, Clp protease, endoprotease (e.g. trypsin), or tRNA synthetase. In some examples, a binding agent may be a cleaving enzyme (e.g., trypsin, endoprotease) that has been modified to remove the peptidase activity. The binding agent may also recognize a terminal amino acid that is attached to a substrate; for example, after all but the final monomer of a polymeric analyte has been coupled to the capture moiety or capture moieties and cleaved, the final monomer may remain coupled to a substrate. Accordingly, the binding agent may recognize and bind the surface-coupled monomer.

The binding agents may be contacted with and specifically bind to cleaved monomers, monomer-linker complex, monomer-linker-capture moiety, or monomer-capture moiety complexes (altogether referred to herein as “monomeric analytes”). For example, a monomeric analyte may fall in any size or range of sizes that is less than that of the entire polymeric analyte. A monomeric analyte complex may be about 0.1 nanometer (nm), about 0.5 nm, 1 about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (μm), about 10 μm, about 100 μm, about 1 millimeter mm in size or greater. The monomeric analyte may have any molecular weight or range of molecular weights. The monomeric analyte may be about 1 dalton (Da), 10 Da, 100 Da, 500 Da, 1 kilodalton (kDa), 10 kDa, 100 kDa, 1,000 kDa, 10,000 kDa, 100,000 kDa, or greater. The monomeric analyte may vary in molecular weight or length, e.g., depending on the amino acid residue.

The binding agent may comprise or be coupled, directly or indirectly, to a polymerizable molecule. The polymerizable molecule may be the same type of molecule as the binding agent (e.g., both peptides, both nucleic acid molecules, etc.), or they may be different. In some instances, the binding agent comprises a peptide (e.g., antibody or antibody fragment) and the polymerizable molecule comprises a nucleic acid molecule. The polymerizable molecule may be conjugated to the binding agent via a chemical conjugation approach, e.g., using linkers such as SMCC, (N-e-maleimidocaproyloxy) succinimide ester (EMCS), succinimidyl-4-(p-maleimidophenyl)butryate (SMPB), succinimidyl-(N-maleimidopropionamido-ethyleneglycol) ester (SMPEG), Succinimidyl (NHS) esters, succinimidyl-4-formylbenzamide (S-4FB), succinidmidyl-6-hydrazino-nicotinamide (S-HyNic), 4-Phenyl-3H-1,2,4-triazoline-3.5(4H)diones (PTAD) or other diazonium, 1-ethyl-3-3-dimethylaminoproyl carbodiimide hydrochloride (EDC), etc. Synthesis of the peptide-nucleic acid molecule conjugate may also be carried out using solid-phase synthesis, fragment conjugation (e.g., using heterobifunctional crosslinkers such as those comprising an aliphatic chain and a maleimide group on one end and NHS on the other), click chemistry (e.g., strain-promoted azide alkyne cycloaddition, inverse-electron-demand Diels-Alder reactions), or combinations of approaches or chemistries. In some instances, the polymerizable molecule may be conjugated to the binding agent using an enzymatic approach. For example, a DNA-protein conjugate may be generated using a truncated a nuclease (e.g., Cas protein such as Cas9), a relaxase (e.g., VirD2), or other enzyme, ribozyme, or DNAzyme. In some instances, the polymerizable molecule may be conjugated to the binding agent using a SpyTag and SpyCatcher interaction, a biotin-avidin interaction, a SNAP-tag, or other interaction. Optional purification may be performed, e.g., using ion-exchange chromatography, HPLC, affinity chromatography, or other purification technique.

The binding agent may be coupled to the polymerizable molecule via a noncovalent interaction. For instance, the binding agent may comprise an avidin or streptavidin tag, to which biotin-conjugated polymerizable molecules can bind. Alternatively, the binding agent may comprise a biotin tag to which an avidin or streptavidin-conjugated polymerizable molecule can bind.

The polymerizable molecule may comprise identifying information of the binding agent. For example, the polymerizable molecule may comprise a nucleic acid barcode molecule comprising a barcode sequence. The barcode sequence may encode for the identity of the binding agent or the binding partner. For example, a monomer (e.g., amino acid) of a polymeric analyte (e.g. peptide comprising a plurality of amino acids) may be cleaved and coupled to the capture moiety (e.g., on a substrate) and may be contacted with a binding agent (e.g., antibody, antibody fragment, nanobody). The binding agent may specifically recognize the amino acid residue or derivative thereof (e.g., a PTH, PTC, ATZ derivatized form) over other amino acid residues or derivatives thereof. The nucleic acid barcode molecule may comprise information that identifies the binding agent, which, due to the specificity of the binding agent to its target, may also identify the particular amino acid residue (or derivative).

The polymerizable molecule of the binding agent may comprise additional multiplexed information. For example, the polymerizable molecule, e.g., a nucleic acid molecule, may comprise sequences that encode cycle or other temporal information or spatial information. In one such example, an array of peptides and capture moieties may be provided on a substrate. The array may comprise a plurality of individually addressable units, in which each (or a subset of) individually addressable units of the array comprises a peptide to be analyzed and a capture moiety. The binding agents, and the polymerizable molecules comprised therein or coupled thereto, may comprise spatial information (e.g., spatial barcode sequences) which uniquely identify the individually addressable units and thus the location of the array. The polymerizable molecules may additionally comprise temporal information (e.g., a cycle barcode that indicates the round or iteration in which the binding agent or polymerizable molecule is provided). Subsequent sequencing of the polymerizable molecule may be used to reveal the spatial information (e.g., the originating location in the array of a peptide or amino acid). In some instances, the polymerizable molecule may comprise a unique molecular identifier (UMI), which may be used to determine the quantity of a given binding agent or monomer (e.g., amino acid) for a given peptide, substrate, array, or sample.

Alternatively, the binding agent that recognizes the monomer-capture moiety complex may not comprise or be coupled to a polymerizable molecule. In such cases, subsequent to the binding of the binding agent to the monomer-capture moiety complex, an additional molecule (e.g., a secondary binding agent) comprising a detectable label, e.g., fluorophore, radioisotope, mass tag, or an identifying polymerizable molecule (e.g., nucleic acid barcode molecule) may be contacted with and bind to the binding agent that is bound to the monomer-capture moiety complex. In some examples, the additional molecule comprises an identifying polymerizable molecule, and the identifying polymerizable molecule may be coupled to or transferred to the additional polymerizable molecule. In one non-limiting example, the binding agent comprises a primary antibody or antibody fragment that recognizes the monomer-capture moiety complex (e.g., terminal amino acid-linker-capture moiety complex) or portion thereof (e.g., the terminal amino acid, or the terminal amino acid-linker complex); subsequent to binding of the primary antibody or antibody fragment to the monomer-capture moiety complex or portion thereof, a secondary antibody or antibody fragment comprising or coupled to a polymerizable molecule (e.g., nucleic acid barcode molecule) is coupled to the primary antibody. The polymerizable molecule of the secondary antibody or antibody fragment may comprise information on the secondary antibody or antibody fragment, the primary antibody or antibody fragment, or other information. Transfer or coupling of the polymerizable molecule of the secondary antibody or antibody fragment to the additional polymerizable molecule can be mediated by any suitable technique, e.g., hybridization of nucleic acid molecules optionally mediated by a splint molecule, click chemistry, or association of high affinity molecules (e.g., streptavidin and biotin).

In some instances, the method may comprise contacting the monomer-capture moiety complex with a library of binding agents. The library of binding agents may comprise a plurality of binding agents that have specificity to different analytes. For example, the library of binding agents may comprise a plurality of binding agents that recognize different amino acids or derivatives thereof (e.g., derivatized amino acids such as the PTH, PTC, or ATZ forms), clusters of amino acids (e.g., dipeptides, tripeptides, etc.), or combinations of amino acids (e.g., amino acids with similar side chain groups). In one such example, a given binding agent may recognize and bind to more than one amino acid, optionally with different affinities or binding kinetics. A given binding agent may recognize and bind to a single amino acid, two different amino acids, three different amino acids, four different amino acids, etc. For instance, a given binding agent may bind to amino acids with similar residues, e.g., amino acids with positively-charged side chains (e.g., arginine, histidine, lysine), negatively-charged side chains (aspartic acid, glutamic acid), amino acids with polar uncharged side chains (e.g., serine, threonine, asparagine, glutamine), amino acids with hydrophobic side chains (e.g., alanine, valine, isoleucine, leucine, methionine, phenylalanine, tyrosine, trytophan), or a combination thereof. Altogether, the library of binding agents may specifically recognize or bind to any number of different amino acids; for example, the library of binding agents may be configured to specifically bind to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different proteinogenic amino acids or derivatives thereof.

The library of binding agents may comprise any useful number of binding agents, each of which can have different binding specificities. For example, a first binding agent may recognize and one amino acid, and second binding agent may recognize two amino acids, and a third binding agent may recognize three amino acids. In another example, a first binding agent may recognize one amino acid, a second binding agent may recognize a different amino acid, and a third binding agent may recognize a plurality of amino acids. It will be appreciated that any number of binding agents may be used and that each binding agent may have specificity to one or more amino acids. Altogether, the library of binding agents may bind to all 20 proteinogenic amino acids or derivatives thereof, or a subset (e.g., 10 or more, 15 or more) of the amino acids.

A binding agent may be passivated prior to or during contact with the cleaved monomer. Passivation may be achieved using a blocking agent or solution, such as milk proteins (e.g., lactoglobulin, lactalbumin, lactoferrin, casein, whey, immunoglobulin, insulin, growth factors, osteopontin), albumin (e.g., bovine serum albumin), Tween 20, commercially available blocking solutions, or a combination thereof. Alternatively, or in addition to, passivation of the binding agent may be performed using a polymer (e.g., polyethylene glycol), organic compound (e.g., oil, lipids), sugar, nanoparticle, inorganic compound, ion, etc.

Additional examples of binding agents that can be used to detect monomers or monomer-linker complexes are provided in International Pat. Pub. No. WO2025/064836, which is incorporated by reference herein in its entirety.

Coupling of Polymerizable Molecules: The polymerizable molecules may be coupled to one another using any useful approach. Such coupling may comprise a covalent interaction or a noncovalent interaction (e.g., ionic interaction, hydrophobic interaction, van der Waals forces, etc.). In some instances, the first polymerizable molecule and the second polymerizable molecule comprise nucleic acid molecules and may be coupled via hybridization, ligation, or both. For instance, the first polymerizable molecule may comprise a first sequence that is complementary to a second sequence of the second polymerizable molecule, and the coupling may occur via hybridization of the first sequence to the second sequence. Alternatively, the first sequence and the second sequence may not be complementary to one another, but may be complementary to a third sequence and a fourth sequence, respectively, of a splint or bridge oligonucleotide. Accordingly, coupling of the first polymerizable molecule to the second polymerizable molecule may be mediated by hybridization of the first and second sequences to the third and fourth sequences, respectively, of the splint or bridge oligonucleotide.

In some instances, a nucleic acid reaction may be performed as part of or in addition to the coupling of the first polymerizable molecule to the second polymerizable molecule. For example, the first sequence of the first polymerizable molecule may hybridize to the second sequence of the second polymerizable molecule, and a nucleic acid extension reaction (e.g., using a polymerase) may be performed. Such an extension reaction may allow for transfer of the encoded information of one of the polymerizable molecules (e.g., the first polymerizable molecule) to another polymerizable molecule (e.g., the second polymerizable molecule). In another example, the first sequence of the first polymerizable molecule may be ligated to the second sequence of the second polymerizable molecule to provide a first polymerizable molecule covalently coupled to the second polymerizable molecule.

In some instances, the first polymerizable molecule and the second polymerizable molecule comprise peptides and are coupled via formation of new peptide (amide) bonds between the C-terminal carboxy and N-terminal amino group of peptides. In some instances, new peptide (amide) bonds are ligated by chemical ligation methods such as native chemical, α-Ketoacid-Hydroxylamine, Staudinger, or Serine threonine ligation. In some instances, new peptide (amide) bonds are formed by enzymes such as transpeptidases, sortases, asparaginyl endoproteases, trypsin related enzymes, or subtilisin-derived variants. Additional examples of chemical and enzymatic ligations of new peptide (amide) bonds are described in Nuijens et al. 2019. Front. Chem. 7:829. doi: 10.3389/fchem.2019.00829, and Kulkarni et al. 2018. Nat Rev Chem 2, 0122 doi: 10.1038/s41570-018-0122, which are both incorporated by reference in their entireties.

The polymerizable molecules may be coupled chemically, either covalently or noncovalently. In some instances, the first polymerizable molecule may be chemically linked to the second polymerizable molecule. For example, the first polymerizable molecule may comprise a first reactive moiety, and the second polymerizable molecule may comprise a second reactive moiety that is capable of reacting with the first reactive moiety. The first reactive moiety may be contacted with the second reactive moiety and be subjected to conditions sufficient to link the first reactive moiety to the second reactive moiety, e.g., via click chemistry. In other instances, the first polymerizable molecule may be coupled to the second polymerizable molecule via a noncovalent or indirect interaction, e.g., biotin-streptavidin.

In some instances, the polymerizable molecule of the binding agent can be coupled to additional polymerizable molecules. For example, a substrate may comprise the polymeric analyte and capture moiety coupled thereto, along with a plurality of additional polymerizable molecules. After coupling of the monomer to the capture moiety and cleavage of the monomer from the polymeric analyte, the monomer may be contacted with the same or different binding agents any number of times. The polymerizable molecule of a single binding agent may contact and be coupled to any number of the additional polymerizable molecules iteratively for repeated interrogation; for instance, the polymerizable molecule of the binding agent may couple to a first additional polymerizable molecule, as described herein, and then subsequently cleaved or removed (e.g., via dehybridization) and contacted and coupled to a second additional polymerizable molecule. Such an approach may be advantageous in transferring several copies of the polymerizable molecule of the binding agent to the substrate.

Fingerprinting: The methods described herein may be useful in complete de novo protein or peptide sequencing (e.g., the identification of each amino acid in a peptide), or for fingerprinting a protein (e.g., identifying only a subset of amino acid types in a peptide and inferring, using a reference database, the identity of the peptide). For fingerprinting, a subset of amino acids may be identified, e.g., using the approaches described herein, without the need of identifying all 20 proteinogenic amino acids. For example, identification of or discrimination between 1, 2, 3, 4, 5, 6, 7, 8, 9, 10, 11, 12, 13, 14, 15, 16, 17, 18, or 19 different amino acids may be sufficient to determine the identity of a protein or peptide. For known proteome databases, reference-based reconstruction may be performed by simulating NGS reads that would be generated from the set of possible peptides in the workflow. For each possible peptide, a simulation can produce NGS reads mimicking the output of this protein sequence system. Next, the real (experimental) NGS reads from a run can be matched to simulated reads from candidate peptides from a database based on likelihood. This results in reconstructed, fragmentary, peptide sequences (contigs) with probability assigned to the assembled peptide sequence.

High Throughput Sequencing/Parallelization: The methods described herein may be conducted in a parallelized, high-throughput format. Such parallelization may be achieved by having substrates comprising multiple polymeric analytes coupled thereto and performing the operations across the substrate.

In some instances, it may be useful to barcode the polymeric analytes prior to processing. Barcode sequences may be attached to the polymeric analytes at a single location (e.g., at a terminus), multiple locations, adjacent to the polymeric analyte (e.g., on a substrate), etc. as is described elsewhere herein. For example, a peptide may be labeled at the N-terminus, C-terminus, or an internal amino acid with a nucleic acid barcode molecule. The nucleic acid barcode sequence may comprise information or be unique to a partition or compartment, sample, peptide, etc. such that each unique barcode sequence can be traced back (e.g., subsequent to nucleic acid sequencing or other detection method) to the originating partition or compartment, sample, peptide, etc.

Alternatively, or in addition to, the capture moieties or polymerizable molecules may comprise a barcode sequence. The barcode sequence may be specific to a particular partition, sample, or spatial location. For example, a substrate may comprise a plurality of individually or by introduction of chaotropic agents (e.g., guanidine, formamide, urea) units. The polymerizable units or capture moieties of each individually addressable unit may comprise a unique barcode specific to the individually addressable unit (e.g., a spatial barcode). The polymeric analytes may be coupled to the substrate such that each individually addressable unit comprises, on average, no more than one polymeric analyte. Such a distribution of polymeric analytes may be obtained, for example, using a limited dilution approach (e.g., diluting the polymeric analytes to reduce the number of polymeric analytes that may attach to a given individually addressable unit) or by introduction of chaotropic agents (e.g., guanidine, formamide, urea). The polymeric analytes may be distributed across the individually addressable units according to a Poisson distribution. Thus, for a given substrate, about 6%, 10%, 18%, 20%, 30%, 36%, 40%, or 50% of the individually addressable units may comprise one or fewer polymeric analytes.

Modifications of Polymeric Analytes and Polymerizable Molecules: The present disclosure also provides for methods of modifying polymeric analytes or monomers of the polymeric analytes (e.g., amino acids of a peptide), as well as polymerizable molecules described herein. Such modifications may useful, for example, in rendering a monomer more resistant to certain reaction conditions (e.g., Edman degradation), to increase or decrease binding affinity of a binding agent to the modified monomer, to assist in docking or interfacing of the modified monomer to an enzyme (e.g., a protease, cleaving enzyme or enzyme analog such as a ribozyme or DNAzyme, binding agent), or other purpose.

A polymeric analyte such as a peptide may be modified in order to render the peptide or a constituent amino acid more resistant to the reaction conditions for cleaving the amino acid from the peptide. For example, the peptide may be subjected to alkylation, e.g., using 4-vinylpyridine, iodoacetamide, which may be useful in preventing oxidation of cysteine residues. The peptide may be subjected to acetylation, e.g., O-acetylation to form an ester such as acetyl chloride, which may be useful in preventing dehydration, racemization, or destruction of a derivatized (e.g., PTH form) of serine or threonine. The peptide may be subjected to β-elimination of a phosphate, followed by a Michael addition of a thiol group, (e.g., as described in Knight et al. 2003. Nature Biotechnology 21, 1047-1054, which is incorporated by reference herein) to detect phosphorylation events. The peptide may be contacted with phenyl isothiocyanate, acetic anhydride, or other amine-reactive group to protect lysine residues. Additional examples of peptide processing for Edman degradation can be found in Tarr, Methods of Protein Microcharacterization. pp 155-194, which is incorporated by reference herein.

A polymeric analyte or monomer may be modified to influence the interaction of a binding agent with the polymeric analyte or monomer, e.g., by derivatizing the cleaved monomer, adding of chemical groups to the cleaved monomer, or other chemical processing (e.g., addition or removal of groups) from the cleaved monomer. Blocking of the binding agent may be achieved by appending a blocking agent, e.g., a chemical group or adduct, to the monomer-capture moiety complex; for example, conjugation a synthetic polymer (e.g., PEG), nucleic acid molecule, fluorophores, quenchers, nanotube, nanoparticle, small molecules, polypeptide or protein, fatty acid chain, or other large, sterically-hindering molecules. The blocking agents may be appended to the monomer using a chemical approach (e.g., reacting with an amino acid, e.g., via a photo-reaction) or enzymatically, e.g., using methyltransferases, tRNA synthetases, acetyltransferases, etc.

Additional examples of modifications to monomers, cleaved monomers, and binding agents can be found in International Patent Publication No. WO2023/196642, which is incorporated by reference herein in its entirety.

Order of Operations: It will be appreciated that the operations presented in the methods described herein may be performed in any useful or convenient order and that some operations, in some instances, may be optional. For example, in some instances, the coupling of the monomer to the capture moiety may occur prior to, during, or subsequent to the cleaving of the monomer from the polymeric analyte. Similarly, the coupling of the sequencing reagent to the capture moiety may occur prior to, during, or subsequent to the coupling of the sequencing reagent to the monomer. In another example, the substrate may be provided with the cleaved monomers coupled thereto, such that cleavage of the monomer from the polymeric analyte is obviated. In yet another example, in instances where a linker is used to couple to the monomer (e.g., amino acid) and the capture moiety or substrate, the linker may comprise a monomer-coupling group and subsequently be reacted with a substrate-binding group (e.g., an oligonucleotide); alternatively, the linker may be provided with the substrate-binding group as part of the linker (e.g., pre-conjugated to the substrate-binding group).

Additional operations may be performed at any useful or convenient step, e.g., prior to provision of the polymeric analyte (e.g., peptide) or subsequent to one or more of the processing operations (e.g., subsequent to coupling of the linker, polymerizable molecules, contacting with binding agents, etc.). For instance, it may be useful to purify or enrich or purify a population of polymerizable molecules. Such enrichment or purification can be performed using any useful technique, e.g., bead-based enrichment, immunoprecipitation, chromatography, electrophoresis, DNA purification, etc. In one such example, purification of a nucleic acid molecule may be performed using a bead comprising or coupled to a complementary sequence of the nucleic acid molecule and optionally, subsequent to capture, eluting the nucleic acid molecule. Similarly, a protein may be purified using a bead comprising an antibody that recognizes the protein or a portion of the protein.

Additional methods, systems, compositions, and kits are contemplated herein. The systems, compositions, and kits may include any useful combination of reagents, analytes, buffer compositions, temperatures, and system components. For example, a system of the present disclosure may comprise a nanopore, a membrane, a sensor, a power source, or any component of a nanopore sequencer. A system may comprise a nanopore and an analyte (e.g., a polymeric analyte, or a modified monomer or stacked plurality of modified monomers derived therefrom), and a reaction mixture. The mixture reaction may comprise one or more monomers, a polymerizing enzyme, and any reagents necessary to perform a polymerization reaction (e.g., cofactors, enzyme substrates, etc.). Additional examples of such methods, systems, compositions, and used for processing polymeric analytes (e.g., peptides) are provided in U.S. Pat. No. 11,971,417; U.S. Pat. Pub. No. 2024/0409995; U.S. Pat. Pub. No. 2025/0155447; U.S. Pat. No. 12,259,393; U.S. Pat. Pub. No. US2023/0104998; U.S. patent application Ser. No. 18/740,088, filed Jun. 11, 2024; International Pat. Pub. No. WO2025/147587; International Pat. Pub. No. WO2025166050; U.S. Prov. Pat. App. No. 63/813,800, filed May 29, 2025; U.S. Prov. Pat. App. No. 63/867,869, filed Aug. 21, 2025; and U.S. Prov. Pat. App. No. 63/825,388, filed Jun. 17, 2025; each of which is incorporated by reference herein in its entirety.

Substrate Conjugation. The present disclosure provides methods for coupling molecules (e.g., biomolecules such as nucleic acid molecules, peptides, lipids, carbohydrates, etc.) to a substrate. The substrate may be functionalized to allow for covalent or noncovalent coupling of the molecules to a substrate. The substrate may comprise any useful functional moiety, e.g., a reactive moiety. In a non-limiting example, a reactive moiety may comprise a click chemistry moieties, such as azide, alkyne, nitrone, alkene (e.g., a strained alkene), tetrazine, methyltetrazine, triazole, tetrazole, phosphite, phosphine, etc. A click chemistry moiety may be reactive in copper-catalyzed Huisgen cycloaddition or the 1,3-dipolar cycloaddition between an azide and a terminal alkyne, Ruthenium-catalyzed azide-alkyne cycloaddition, a Diels-Alder reaction (e.g., a cycloaddition between a diene and a dienophile), or a nucleophilic substitution reactions in which one of the reactive species is an epoxy or aziridine. A molecule that is to be coupled to a substrate may comprise a complementary click chemistry moiety to that of the substrate; for example, the substrate may comprise an alkyne moiety and the molecule to be coupled may comprise an azide moiety, which can react with the alkyne moiety of the substrate to generate a covalent linkage. In one such example, the substate may comprise dibenzocyclooctyne (DBCO) moieties to which azide-comprising molecules (e.g., azide-DNA, azide-polymers, azide-peptides) can react and conjugate.

The reactive moiety may comprise a photoreactive moiety that may be activated when exposed to a photostimulus (e.g., light such as UV or visible light). Examples of photoreactive moieties include aryl (phenyl) azides (e.g., phenyl azide, ortho-hydroxyphenyl azide, meta-hydroxyphenyl azide, tetrafluorophenyl azide, ortho-nitrophenyl azide, meta-nitrophenyl azide), diazirines, azido-methyl-coumarins, benzophenones, anthraquinones, diazo compounds, diazirines, psoralen, and analogs or derivatives thereof.

The reactive moiety may comprise a carboxyl-reactive crosslinker group, such as diazomethane, diazoacetyl, carbonyldiimidazole, carbodiimides (e.g., 1-ethyl-3-(3-dimethylaminopropyl) carbodiimide hydrochloride (EDC)), dicyclohexylcarbodiimide (DCC)), or an amine-reactive group (e.g., N-hydroxysulfosuccinimide (NHS), Sulfo-NHS, or NHS-esters). The reactive group may comprise a crosslinking agent, which may comprise an NHS group, an EDC group, a maleimide, a thiol, a cystamine, an aldehyde, a succinimidyl group, an expoxide, an acrylate. Examples of crosslinking agents include, for example, NHS (N-hydroxysuccinimide); sulfo-NHS (N-hydroxysulfosuccinimide); EDC (1-Ethyl-3-[3-dimethylaminopropyl]); carbodiimide hydrochloride; SMCC (succinimidyl 4-(N-maleimidomethyl)cyclohexane-1-carboxylate); DSS (disuccinimidyl suberate); DSG (disuccinimidyl glutarate); DFDNB (1,5-difluoro-2,4-dinitrobenzene); BS3 (bis(sulfosuccinimidyl)suberate); TSAT (tris-(succinimidyl)aminotriacetate); BS(PEG)5 (PEGylated bis(sulfosuccinimidyl)suberate); BS(PEG)9 (PEGylated bis(sulfosuccinimidyl)suberate); DSP(dithiobis(succinimidyl propionate)); DTSSP (3,3′-dithiobis(sulfosuccinimidyl propionate)); DST(disuccinimidyl tartrate); BSOCOES (bis(2-(succinimidooxycarbonyloxy)ethyl) sulfone); EGS (ethylene glycol bis(succinimidyl succinate)); DMA (dimethyl adipimidate); DMP (dimethyl pimelimidate); DMS (dimethyl suberimidate); DTBP (Wang and Richard's Reagent); BM (PEG)2 (1,8-bismaleimido-diethyleneglycol); BM (PEG)3 (1,11-bismaleimido-triethyleneglycol); BMB (1,4-bismaleimidobutane); DTME (dithiobismaleimidoethane); BMH (bismaleimidohexane); BMOE (bismaleimidoethane); TMEA (tris(2-maleimidoethyl)amine); SPDP (succinimidyl 3-(2-pyridyldithio)propionate); SMCC (Succinimidyl trans-4-(maleimidylmethyl)cyclohexane-1-Carboxylate); SIA (succinimidyl iodoacetate); SBAP (succinimidyl 3-(bromoacetamido)propionate); STAB (succinimidyl (4-iodoacetyl)aminobenzoate); Sulfo-SIAB (sulfosuccinimidyl (4-iodoacetyl) aminobenzoate); AMAS (N-α-maleimidoacet-oxysuccinimide ester); BMPS (N-β-maleimidopropyl-oxysuccinimide ester); GMBS (N-γ-maleimidobutyryl-oxysuccinimide ester); Sulfo-GMBS (N-γ-maleimidobutyryl-oxysulfosuccinimide ester); MBS (m-maleimidobenzoyl-N-hydroxysuccinimide ester); Sulfo-MBS (m-maleimidobenzoyl-N-hydroxysulfosuccinimide ester); SMCC (succinimidyl 4-(N-maleimidomethyl)cyclohexane-1-carboxylate); Sulfo-SMCC (sulfosuccinimidyl 4-(N-maleimidomethyl)cyclohexane-1-carboxylate); EMCS (N-8-malemidocaproyl-oxysuccinimide ester); Sulfo-EMCS (N-α-maleimidocaproyl-oxysulfosuccinimide ester); SMPB (succinimidyl 4-(p-maleimidophenyl)butyrate); Sulfo-SMPB (sulfosuccinimidyl 4-(N-maleimidophenyl)butyrate); SMPH (Succinimidyl 6-((beta-maleimidopropionamido)hexanoate)); LC-SMCC (succinimidyl 4-(N-maleimidomethyl)cyclohexane-1-carboxy-(6-amidocaproate)); Sulfo-KMUS (N-κ-maleimidoundecanoyl-oxysulfosuccinimide ester); SPDP (succinimidyl 3-(2-pyridyldithio)propionate); LC-SPDP (succinimidyl 6-(3 (2-pyridyldithio)propionamido)hexanoate); LC-SPDP (succinimidyl 6-(3 (2-pyridyldithio)propionamido)hexanoate); Sulfo-LC-SPDP (sulfosuccinimidyl 6-(3′-(2-pyridyldithio)propionamido)hexanoate); SMPT (4-succinimidyloxycarbonyl-alpha-methyl-α(2-pyridyldithio) toluene); PEG4-SPDP (PEGylated, long-chain SPDP crosslinker); PEG12-SPDP (PEGylated, long-chain SPDP crosslinker); SM(PEG)2 (PEGylated SMCC crosslinker); SM(PEG)4 (PEGylated SMCC crosslinker); SM(PEG)6 (PEGylated, long-chain SMCC crosslinker); SM(PEG)8 (PEGylated, long-chain SMCC crosslinker); SM(PEG)12 (PEGylated, long-chain SMCC crosslinker); SM(PEG)24 (PEGylated, long-chain SMCC crosslinker); BMPH (N-β-maleimidopropionic acid hydrazide); EMCH (N-ε-maleimidocaproic acid hydrazide); MPBH (4-(4-N-maleimidophenyl) butyric acid hydrazide); KMUH (N-κ-maleimidoundecanoic acid hydrazide); PDPH (3-(2-pyridyldithio) propionyl hydrazide); ATFB-SE (4-Azido-2,3,5,6-Tetrafluorobenzoic Acid, Succinimidyl Ester); ANB-NOS (N-5-azido-2-nitrobenzoyloxysuccinimide); SDA (NHS-Diazirine) (succinimidyl 4,4′-azipentanoate); LC-SDA (NHS-LC-Diazirine) (succinimidyl 6-(4,4′-azipentanamido)hexanoate); SDAD (NHS-SS-Diazirine) (succinimidyl 2-((4,4′-azipentanamido)ethyl)-1,3′-dithiopropionate); Sulfo-SDA (Sulfo-NHS-Diazirine) (sulfosuccinimidyl 4,4′-azipentanoate); Sulfo-LC-SDA (Sulfo-NHS-LC-Diazirine) (sulfosuccinimidyl 6-(4,4′-azipentanamido)hexanoate); Sulfo-SDAD (Sulfo-NHS-SS-Diazirine) (sulfosuccinimidyl 2-((4,4′-azipentanamido)ethyl)-1,3′-dithiopropionate); SPB (succinimidyl-[4-(psoralen-8-yloxy)]-butyrate); Sulfo-SANPAH (sulfosuccinimidyl 6-(4′-azido-2′-nitrophenylamino)hexanoate); DCC (dicyclohexylcarbodiimide); EDC (1-ethyl-3-(3-dimethylaminopropyl) carbodiimide hydrochloride); gluteraldehyde; formaldehyde; and combinations or derivatives thereof.

Molecules may also be attached to substrates using linkers. The linkers can have any useful number of functional groups or reactive groups and may be uni-functional (having one functional group), bi-functional, tri-functional, quadri-functional, or comprise a greater number of functional groups. In some instances, a molecule (e.g., nucleic acid molecule, peptide, or polymer) may be attached to a substrate using a heterobifunctional linker. The heterobifunctional linker may comprise any useful functional group, as described herein. Non-limiting examples of heterobifunctional linkers include: p-Azidobenzoyl hydrazide (ABH), N-5-Azido-2-nitrobenzoyloxysuccinimide (ANB-NOS), N-[4-(p-Azidosalicylamido)butyl]-3′-(2′-pyridyldithio) propionamide (APDP), p-Azidophenyl Glyoxal monohydrate (APG), Bis [B-(4-azidosalicylamido)ethyl]disulfide (BASED), Bis [2-(Succinimidooxycarbonyloxy)ethyl] Sulfone (BSOCOES), BMPS, 1,4-Di [3′-(2′-pyridyldithio)propionamido] Butane (DPDPB), Dithiobis(succinimidyl Propionate) (DSP), Disuccinimidyl Suberate (DSS), Discuccinimidyl Tartrate (DST), 3,3′-Dithiobis(sulfosuccinimidyl Propionate (DTSSP), EDC, Ethylene Glycol bis (succinimidyl succinate) (EGS), N-(E-maleimidocaproic acid hydrazide (EMCH), N-(E-maleimidocaproyloxy)-succinimide ester (EMCS), N-Maleimidobutyryloxysuccinimide ester (GMBS), Hydroxylamine-HCl, MAL-PEG-SCM, m-Maleimidobenzoyl-N-hydroxysuccinimide Ester (MBS), N-Hydroxysuccinimidyl-4-azidosalicylic acid (NHS-ASA), PDPH, N-Succinimidyl bromoacetate (SBA), SIA, Sulfo-SIA, Succinimidyl-4-(N-maleimidomethyl)cyclohexane-1-carboxylate (SMCC), Succinimidyl 4-(p-maleimidophenyl) Butyrate (SMPB), Succinimidyl-6-[β-maleimidopropionamido]hexanoate (SMPH), N-Succinimidyl 3-[2-pyridyldithio]-propionate (SPDP), Sulfo-LC-SPDP, N-(p-Maleimidophenyl isocyanate (PMPI), N-Succinimidyl (4-iodoacetyl) Aminobenzoate (SIAB), Sulfo-MBS, Sulfo-SANPAH, Sulfo-SMCC, Sulfo-DST, Sulfo-EMCS, Sulfo-GMBS, N-Hydroxysulfosuccinimidyl-4-azidobenzoate (Sulfo-HSAB), Sulfosuccinimidyl (4-azidophenyl)-1,3 dithio propionate (Sulfo-SADP), Sulfosuccinimidyl 2-(m-azido-o-nitrobenzamido)-ethyl-1,3′-dithio propionate (Sulfo-SAND), Sulfosuccinimidyl-2-(p-azidosalicylamido)ethyl-1,3-dithiopropionate (Sulfo SASD), Sulfo-SIAB, Sulfo-SMCC, Sulfo-SMPB, and the like.

Additional examples of conjugation reactions that may be used to attach molecules to one another or to substrates include Castro-Stevens coupling, Larock indole synthesis, Miyaura borylation, Sonagashira cross-coupling, a Grubbs reaction, a Diels-Alder reaction, Staudinger ligation, Oxime ligation, Hydrazone formation, Thiol-ene reaction, Thiol-yne reaction, Thiol-maleimide reaction, Thiol-bromomaleimide reaction, Thiol-haloacetyl reaction, Disulfide formation, Thioether formation, Suzuki coupling, Sonogashira coupling, Heck reaction, Buchwald-Hartwig amination, Chan-Lam coupling, Negishi coupling, Kumada coupling, Stille coupling, Hiyama coupling, Ullmann coupling, Cadiot-Chodkiewicz coupling, Glaser coupling, Wurtz coupling, Williamson ether synthesis, Mitsunobu reaction, Baylis-Hillman reaction, Passerini reaction, Ugi reaction, Biginelli reaction, Hantzsch pyridine synthesis, Knoevenagel condensation, Aldol condensation, Claisen condensation, Horner-Wadsworth-Emmons reaction, Wittig reaction, Julia olefination, Peterson olefination, Robinson annulation, Paal-Knorr synthesis, Pictet-Spengler reaction, Bischler-Napieralski reaction, Vilsmeier-Haack reaction, Reductive amination, Mannich reaction, Michael addition, Friedel-Crafts acylation, Friedel-Crafts alkylation, Grignard reaction, Barbier reaction, Nozaki-Hiyama-Kishi reaction, Petasis reaction, Ritter reaction, Sharpless asymmetric epoxidation, Sharpless asymmetric dihydroxylation, Jacobsen epoxidation, Shi epoxidation, Proline-catalyzed aldol reaction, Barton-McCombie deoxygenation, Birch reduction, Wolff-Kishner reduction, Clemmensen reduction, Mozingo reduction, or Rosenmund reduction.

A molecule can be attached to the substrate via a releasable or labile linker. The molecule may be releasable from the substrate upon the application of a stimulus. In some cases, the stimulus may be a photo-stimulus, e.g., through cleavage of a photo-labile linkage that releases the molecule. In other cases, a thermal stimulus may be used, where elevation of the temperature results in cleavage or reversibility of a linkage. For example, the thermally labile bond may include a nucleic acid hybridization attachment, such that thermal melting of the hybrid releases one oligonucleotide from a substrate-bound oligonucleotide. In other embodiments, a chemical stimulus is used that cleaves a linkage of the molecule to the substrate, or otherwise results in release of the molecule from the substrate. For example, the substrate and molecule may be attached via a disulfide bond, and exposure to a reducing agent, such as DTT or TCEP results in release of the molecule from the substrate. In some embodiments, the molecule may be releasable from the substrate using a biological stimulus. For example, a nucleic acid molecule comprising a uracil or restriction site can be cleaved by application of the appropriate enzyme (e.g., nickase, UDG, USER, restriction enzyme, etc.). Other non-limiting examples of labile bonds include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

More than one type of molecule may be coupled to the substrate. For example, a substrate may be coupled to nucleic acid molecules and peptides. Alternatively, a substrate may be coupled to only one type of molecule (e.g., only nucleic acid molecules, only peptides, only lipids, only carbohydrates, etc.). A substrate may be coupled to any useful combination of molecules, linkers, reactive moieties or functional groups, which may be coupled at any useful density, as described elsewhere herein. For example, a multifunctional linker may be used to attach both a nucleic acid barcode molecule and a peptide to the substrate. Alternatively, a substrate may comprise a linker and reactive sites; the linker may be used to attach one type of molecule (e.g., peptides or nucleic acid molecules), whereas the reactive sites may be used to attach another type of molecule (e.g., nucleic acid molecules or peptides).

The proximity of a molecule coupled to a substrate to its nearest neighbor (e.g., another molecule) may be controlled using a variety of approaches, e.g., self-assembling monolayers, patterning approaches, linking moieties, etc. In some instances, it may be advantageous to have two molecules in close proximity (e.g., two polymerizable molecules, such as a peptide and a nucleic acid molecule, or two nucleic acid molecules). For instance, with respect to the sequencing approaches described herein, capture moieties may be used to couple a monomer of a polymeric analyte, and subsequent to monomer cleavage, additional polymerizable molecules may be required to be in proximity to the capture moiety to allow for transfer of information encoded by polymerizable molecules of binding agents. The proximity of the molecules (e.g., capture moiety and polymerizable molecules) may be mediated using tethering molecules, such as nucleic acid molecule “staples” or multi-functional linkers.

The distance between a molecule coupled to a substrate from a surface of the substrate may be modulated, e.g., via a linker. In some instances, the distance of an end of a molecule to a surface of a substrate may be about 0.1 nanometer (nm), about 0.5 nm, about 1 nm, about 2 nm, about 3 nm, about 4 nm, about 5 nm, about 6 nm, about 7 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, or about 1000 nm. The distance of an end of a molecule to a surface of a substrate or the distances of a plurality of ends of molecules to one or more surfaces of one or more substrates may fall in a range of values, e.g., from about 5 nm to about 100 nm, from about 1 nm to about 1 micrometer, etc.

Nucleic acid molecules may be coupled to a substrate by direct coupling. In such instances, the substrate or the nucleic acid molecules may comprise functional moieties that can interact. For example, the substrate and nucleic acid molecules may comprise a complementary click chemistry pair, e.g., alkyne and azide. In one such example, a substrate may comprise alkyne moieties (e.g., DBCO), which can be reacted with azide-functionalized nucleic acid molecules. The nucleic acid molecules may be reacted with the alkyne moieties in a click chemistry reaction to covalently link the substrate to the nucleic acid molecules. In another example, the substrate may comprise avidin or streptavidin moieties, to which biotinylated nucleic acid molecules may interact and bind noncovalently. Alternatively, or in addition to, the substrate may comprise a nucleic acid molecule to which additional nucleic acid molecules (e.g., nucleic acid analytes, nucleic acid linkers) are conjugated using hybridization, ligation, click chemistry, crosslinking (e.g., photocrosslinking such as CNVK).

Alternatively, or in addition to, the nucleic acid molecules may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized DNA primers (e.g., DBCO-DNA primers) may be attached using a linker such as azidoacetic acid NHS ester. In another example, amine-functionalized substrates may be coupled to azide-functionalized DNA primers using a DBCO-NHS ester or DBCO-PEG-NHS ester linker.

Similarly, peptides may be coupled to a substrate by direct coupling or by using a linker. A peptide may be coupled to a substrate at a terminus of the peptide (e.g., C terminus or N terminus), at an internal residue or amino acid of the peptide, or at multiple locations along the peptide. In examples of direct coupling, a peptide may be functionalized with a moiety that can interact with a moiety of the substrate (e.g., click chemistry pair, avidin-biotin). For example, the substrate and peptides may comprise a complementary click chemistry pair, e.g., alkyne and azide, or binding partners such as avidin and biotin. In one example of a click chemistry pair, a substrate may comprise alkyne moieties (e.g., DBCO), which can be reacted with azide-functionalized peptides. The peptides may be reacted with the alkyne moieties in a click chemistry reaction to covalently link the substrate to the peptides. In another example, the substrate may comprise avidin or streptavidin moieties, to which biotinylated peptides may interact and bind non-covalently.

Peptides may be coupled to a substrate via indirect coupling. For instance, a peptide may be coupled to a polymerizable molecule (e.g., an additional peptide or protein, a nucleic acid molecule, a polymer) that can couple to a capture moiety on a substrate (e.g., a binding protein, an additional nucleic acid molecule). In one such example, a peptide may comprise or be coupled to a nucleic acid molecule, and a substrate comprising a complementary or additional nucleic acid molecule may be provided; attachment of the peptide to the substrate can occur via ligation, hybridization (directly or via a splint oligonucleotide), or a nucleic acid extension reaction.

Alternatively, or in addition to, the peptides may be coupled to a substrate using a linker, e.g., as described elsewhere herein. The linker may comprise at least two functional groups (e.g., a heterobifunctional linker) that can couple to both the substrate and the peptides. In an example, the substrate may comprise an amine group, and alkyne-functionalized peptides may be attached using a linker such as azidoacetic acid NHS ester. In another example, amine-functionalized substrates may be coupled to azide-functionalized peptides using a DBCO-NHS ester or DBCO-PEG-NHS ester linker. In yet another example, substrates comprising an amine group may be coupled to an azide-functionalized peptide using EDC and Sulfo-NHS.

A peptide may be functionalized with a functional moiety to enable attachment or coupling of the peptide to the substrate. The functional moiety may comprise a silane, e.g., aminosilane (e.g., APTES), amino-PEG-silane, polymerizable molecule (e.g., nucleic acid molecule) click chemistry moiety or other linking moiety and can be attached to the peptide at a peptide terminus (N-terminus or C-terminus), at an internal amino acid, or at multiple locations (e.g., multiple internal amino acids, one or both termini, etc.). Chemical approaches to functionalize peptides can include C-terminal-specific conjugation (e.g., via C-terminal decarboxylative alkylation) using photoredox catalysis, e.g., as described by U.S. patent application Ser. No. 18/282,288, filed Mar. 25, 2022; Bloom et al, Nature Chemistry 10, 205-211. 2018; and Zhang et al, ACS Chem. Biol. 2021, 16, 11, 2595-2603, each of which is incorporated by reference herein in its entirety, or amide coupling to an amine-functionalized surface. N-terminal attachment may comprise amide coupling of the N-terminus amine group to a carboxylic group functionalized surface or using 2-pyridinecarboxaldehyde variants. Alternatively, or in addition to, functionalization of terminal ends of peptides may be achieved enzymatically or using enzyme analogs such as ribozymes or DNAzymes. In an example of enzymatic functionalization and attachment, carboxypeptidases or amidases are used for C-terminal functionalization (e.g., as described in Xu et al, ACS Chem Biol. 2011 Oct. 21; 6 (10): 1015-1020; Zhu et al, Chinese Chemical Letters. 2018, Vol 29 Issue 7, Pages 1116-1118; and Zhu et al, ACS Catal. 2022, 12, 13, 8019-8026, each of which is incorporated by reference herein in its entirety), which can allow for the addition of a click chemistry moiety to the peptide. The click chemistry-functionalized peptides may then be directly attached to the substrate via another clickable group (e.g., BCN-azide or DBCO-azide coupling), or, in other instances, may be reacted with another linker or polymerizable molecule (e.g., a bait nucleic acid molecule with a clickable group) that can then link to the substrate directly or indirectly (e.g., using a capture nucleic acid molecule and hybridizing, ligating, or extending the bait nucleic acid molecule). Additional examples of enzymes that can be used for functionalization or attachment include Sortase A, subtiligase, Butelase I, or trypsiligase. In some examples, ubiquitin ligase can be used to attach ubiquitin proteins with linker moieties to substrates. These linker moieties can then be used to chemically attach proteins to ubiquitin-coupled substrates. In some examples, glycosylating enzymes may be used to conjugate functionalized sugar groups (e.g., click chemistry functionalized sugars, polymer-conjugated sugars, biotinylated sugars) to amino acid residues, which can allow for attachment to a substrate (e.g., via click chemistry, polymer crosslinking or nucleic acid hybridization, avidin-biotin interactions), etc. Internal amino acid residues or post-translationally modified residues may be coupled to substrates using, for example, thiol labeling, amide coupling using EDC/NHS chemistry or DMT-MM to glutamate or aspartate residues, esterifying glutamate or aspartate residues, alkylation or disulfide bridge labeling of cysteines, or amide coupling to lysine residues.

A peptide may be treated prior to, during, or subsequent to coupling of the peptide to a substrate. In some instances, a peptide is conjugated with a tag that enables attachment to the substrate, e.g., using His tags, SNAP-tags, CLIP-tags, SpyCatcher, SpyTag, nucleic acid tags (e.g., bait oligos which can attach to capture oligos of the substrate). In some examples, it may be advantageous to block or protect primary amines or carboxyl groups and optionally, de-block or de-protect the N-terminus primary amine or C-terminus carboxy group in order to facilitate attachment of the N-terminus or C-terminus to a substrate. In an example, single-point (e.g., C-terminal) selective attachment of peptides can be achieved by reacting the peptide with a linker comprising an amine-reactive group (e.g., isothiocyanates such as PITC) and a reactive group (e.g., click chemistry group). The linker can be, for example, PITC-conjugated click chemistry moieties such as PITC-azide, PITC-alkyne, optionally with spacer moieties in between, e.g., PITC-alkyl-azide, PITC-PEG-azide, PITC-alkyl-alkyne, PITC-PEG-azide). The linker reacts with and “blocks” the primary amines (e.g., modifies lysines), including the N-terminus. Subsequent cleavage of the N-terminal amino acid (e.g., using an Edman reaction, such as application of acid), can be performed, and one of the remaining modified lysines may be attached to a substrate (e.g., using the click chemistry moiety coupled to the amine-reactive group, which can react with a click-functionalized substrate or a click-functionalized polymerizable molecule (e.g., nucleic acid molecule) which can then couple to a capture moiety of a substrate). Optionally, the peptide may be treated with a protease, e.g., LysC, which cleaves peptides such that a remaining peptide has a C-terminal lysine and such that the remaining peptide comprises a primary amine only at the C-terminal lysine residue and the N-terminus; such a cleavage/digestion may be performed prior to reacting the amine-reactive group, e.g., as shown by Xie et al. Langmuir 2022, 38, 30, 9119-9128, which is incorporated by reference herein in its entirety, or the cleavage/digestion may be performed concurrently or subsequent to reacting the amine-reactive group. Attachment of the peptide to the substrate may be performed at any useful or convenient step, e.g., prior to, during, or subsequent to N-terminal cleavage.

In another example, the peptide may be treated with ArgC, which cleaves peptides such that a remaining peptide has a C-terminal arginine. The C-terminal arginine may then be functionalized (e.g., with a click chemistry moiety) using an arginine-reactive group, e.g., a dicarbonyl compound. In yet another example, the peptide may be treated with AspN, which cleaves peptides such that a remaining peptide has an N-terminal aspartic acid residue. The N-terminal aspartic acid may then be functionalized (e.g., with a click chemistry moiety) using an carboxy-specific chemistry. Additional examples of chemical functionalization chemistries for a plurality of amino acid residues are reviewed in N. Kjærsgaard et al. 2022. Chem Bio Chem. 23, e202200245, which is incorporated by reference herein in its entirety.

In some embodiments, attachment of a peptide to a substrate may be facilitated using one or more nucleic acid molecules. For instance, a linker comprising an amine-reactive group (e.g., isothiocyanates such as PITC) and a reactive group (e.g., click chemistry group) may be coupled to the peptide (e.g., at an N-terminus, a lysine side chain); and an oligonucleotide comprising an additional reactive group (e.g., click chemistry group) can react with that of the linker, thereby coupling the oligonucleotide to the peptide via the linker. The oligonucleotide may then couple to a substrate (e.g., bead, flow cell) which comprises an anchor oligonucleotide, e.g., via ligation, extension, hybridization, or other nucleic acid reaction.

Similarly, carboxylic groups can be reacted in a way to enable C-terminal or internal residue attachment. In an example of C-terminal conjugation, carboxyl groups may be labeled with a C-terminal sequencing reagent, such as isothiocyanate, when treated with an activating reagent (e.g., acetic anhydride) to generate a peptide-thiohydantoin (at the C-terminus) and “blocked” carboxyl groups on the aspartic acid and glutamic acid residues. The thiohydantoin may then be reacted to couple to a substrate. Alternatively, cleavage of the C-terminal amino acid via a single round of C-terminal sequencing degradation, or via a protease, exposes only a single reactive carboxylic group at the C-terminal amino acid. The single reactive C-terminal carboxylic group can then be used as a reactive moiety for a single attachment site.

In another approach, a peptide or protein can be attached via the N-terminus using the specific reactivities of the N-terminus amine group. Amine-based reactions, such as amide coupling, can be carried out at low pH where only the N-terminal amine group is active. In addition, 2-pyridinecarboxyaldehyde and variants can be used to react to the N-terminal amine group.

In some instances, a peptide may be conjugated to a substrate using a polymerization reaction, e.g., a free radical polymerization, such as using PEGylated peptides, methacrylamide-modified peptides, Michael-type addition of maleimide-terminated oligo-NIPAAM-conjugated peptides; photocrosslinking of azophenyl-conjugated peptides, or other polymerization reactions with monomer-conjugated peptides, e.g., as described by Krishna et al. Biopolymers. 2010; 94 (1): 32-48, which is incorporated by reference herein in its entirety.

Multiple types of molecules may be attached to a substrate. The substrate may comprise, coupled thereto, any combination of molecules, including but not limited to: peptides, proteins (e.g., enzymes, antibodies, nanobodies, antibody fragments), nucleic acid molecules, lipids, carbohydrates or sugars, metabolites, small molecules, polymers, metals, viral particles, biotin, avidin, streptavidin, neutravidin, etc. The multiple types of molecules may be attached simultaneously to the substrate or in a sequential manner. For example, a substrate may be treated to conjugate nucleic acid molecules and subsequently treated to conjugate peptides, or alternatively, the substrate may be treated to conjugate peptides prior to the nucleic acid molecules. Any number of conjugation or attachment chemistries may be used. For example, in instances where multiple types of molecules are attached to the substrate, any number of conjugation chemistries may be used for each type of molecule.

A substrate, or portion thereof, may be subjected to conditions sufficient to passivate the substrate or portion thereof. Passivation of a substrate may be useful for a variety of purposes, such as preventing nonspecific binding of binding agents, altering the surface density of a molecule (e.g., increasing the density of nucleic acid molecules or peptides), blocking reactive sites (e.g., blocking available click chemistry moieties subsequent to conjugation of the molecules on the substrate), etc. Passivation may be achieved using chemical approaches, e.g., deposition of blocking agents such as proteins (e.g., albumin), Tween-20, polymers, metals or metal oxides, or biochemical approaches, e.g., using metal microbes. Substrates comprising reactive moieties may also be passivated following molecule conjugation (e.g., coupling of nucleic acid molecules, peptides, etc.) by reacting any unreacted sites with an appropriate molecule. For example, a substrate comprising click chemistry moieties, e.g., DBCO beads, may be coupled to molecules of interest (e.g., polymerizable molecules, such as nucleic acid molecules, peptides) at a useful density using click chemistry (e.g., azide-nucleic acid molecules, azide-peptides). Unreacted sites may be passivated by providing and reacting complementary click-chemistry molecules, e.g., azide-polymers (e.g., PEG-azide), which may reduce downstream nonspecific interactions.

Substrate passivation may occur at any useful time or step. For instance, passivation to block unreacted DBCO sites may be performed prior to, during, or subsequent to conjugation of analytes or other molecules of interest (e.g., peptides and nucleic acid molecules). The passivation may be controlled by stoichiometry or densities of the passivating agent relative to the molecules of interest, or by physical approaches, e.g., photopatterning, self assembling monolayers, etc.

Sample Processing. The present disclosure also provides systems, compositions, devices, and methods for processing samples. One or more methods for processing samples may comprise preparation of biological samples for analysis, which, in some instances, includes partitioning of cells for conducting single-cell analysis. A method for processing a biological sample may comprise extraction or isolation of one or more peptides or proteins from the biological sample for further processing and analysis, as is described elsewhere herein.

Preparation of Cell Suspensions for Single-Cell Analysis: The methods described herein may involve preparation of single cell suspensions from a biological sample. Single cell suspensions may be prepared from biological samples by dissociating cells and optionally, culturing them in a liquid medium. In some instances, biological samples comprise a liquid sample. For example, a biological sample may comprise a bacterial liquid culture, a mammalian liquid culture, a blood, plasma, or serum sample. Processing of such liquid samples may include centrifugation (e.g., to isolate cells), resuspension of cells in a suitable medium, such as Dulbecco's Phosphate Buffered Saline (DPBS), and optional culturing of the isolated cells.

A biological sample may comprise cultured cells, e.g., cell cultured in suspension, or cells adhered to a solid surface, such as petri dishes or tissue culture dishes. Cultured adherent cells samples may be treated to generate a cell suspension, e.g., via a protease such as trypsin, to detach the cells from the surface. A biological sample may comprise a tissue or biopsy sample. A tissue or biopsy sample may be processed mechanically or enzymatically to generate a cell suspension. Such processing may include sonication (mechanical treatment) or enzymatic treatment, such as the use of pronase, collagenase, hyaluronidase, metalloproteinases, trypsin, or other enzymes that digest extracellular matrix components. The dissociated cells can then be stored in a suitable buffer, such as DPBS.

Cell Sorting: A biological sample or a cell suspension may be subjected to sorting to isolate a cell of interest. Sorting may be performed to select or isolate a cell based on a quality or characteristic of the cell, e.g., expression of a protein target, size, deformability, fluorescence or other optical property, or other physical property of the cell. Sorting may accomplished using any number of approaches, e.g., using immunosorting (e.g., fluorescence activated cell sorting (FACS) or magnetic activated cell sorting (MACS)), electrophoretic approaches, chromatography, microfluidic approaches (e.g., using inertial focusing, cell traps, electrophoresis, isoelectric focusing), acoustic sorting, optical sorting (e.g., optoelectronic tweezers), mechanical cell picking (e.g., using manual or robotic pipettes) or passive approaches (e.g., gravitational settling).

Partitioning: Cells of a biological sample or cell suspension may be partitioned into individual partitions such that at least a subset of the individual partitions comprises a single cell. The individual partitions may comprise a barcode molecule (e.g., fluorophore or set of fluorophores, nucleic acid barcode molecules, etc.). Barcode molecules may be unique to the partition, such that each individual partition comprises a different barcode sequence than other partitions. The barcode molecules may be loaded into the individual partitions at any useful ratio of barcode molecules to sample species (e.g., cells, proteins, nucleic acid molecules). The barcode molecules may be loaded into partitions such that about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded into partitions such that more than about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded in the partitions so that less than about 0.0001, 0.001, 0.1, 1, 2, 3, 4, 5, 6, 7, 8, 9, 10, 20, 50, 100, 500, 1000, 5000, 10000, or 200000 barcodes are loaded per sample species.

A partition may assume any useful geometry such as a droplet, a microwell, a solid substrate, a gel (e.g., a cell encapsulated in a gel bead), a bead, a flask, a tube, a spot, a capsule, a channel, a chamber, or other compartment or vessel. A partition may be part of an array of partitions, e.g., a droplet in a microfluidic device, a microwell of a microwell plate, a spot on a multi-spot array, etc.

Lysis, Permeabilization, and Analyte Extraction: Single cells (e.g., in partitions) may be processed to obtain one or more analytes contained therein. A method for processing a single cell may comprise lysing the cell to release the contents into the individual compartment or partition. Lysis may be performed using a detergent (e.g., Triton-X 100, sodium dodecyl sulfate, sodium deoxycholate, CHAPS), RIPA buffer, a change in temperature (e.g., elevated or lower temperature, freezing, freeze-thawing), enzymes, mechanical lysis (e.g., sonication, application of mechanical force), electrical lysis, or a combination thereof. Lysis may be performed in the presence of protease inhibitors to prevent degradation or digestion of the proteins from the cell. The contents may optionally be further processed, e.g., subjected to purification or extraction, denaturation of proteins or peptides, enzyme or chemical digestion, etc. In some instances, the contents may be subjected to enzymatic digestion to remove nucleic acid molecules, e.g. using nucleases such as DNAse or RNAse. Alternatively or in addition to, a cell may be fixed (e.g., using a fixative) and/or permeabilized. Examples of fixatives include aldehydes (e.g., glutaraldehyde, formaldehyde, paraformaldehyde), alcohols (e.g., methanol, ethanol), acetone, acids (e.g., acetic acid, Davidson's AFA), oxidizing agents (e.g., osmium tetroxide, potassium dichromate, chromic acid, permanganate salts), Zenker's fixative, picrates, Hepes-glutamic acid buffer-mediated organic solvent protection effect (HOPE), or Karnovsky fixative. Cell permeabilization may be achieved mechanically (e.g., using sonication, electroporation, shearing) or chemically (e.g., using an organic solvent such as methanol or acetone or detergents such as saponin, Tween-20, Triton X-100).

Protein Processing: The biological sample (or single cell suspensions or partitioned cells) may be further processed to enable proteomic analysis. For example, de-aggregation of proteins in the sample may be performed, e.g., using chemical or mechanical approaches. Chemical de-aggregation methods can include but are not limited to: sodium dodecyl (SDS), Triton-X 100, 3-((3-cholamidopropyl)dimethylamminio)-1-proppanesulfonate (CHAPS), ethylene carbonate, or formamide. Mechanical de-aggregation methods can include but are not limited to: sonication or high temperature treatment. The biological sample (or single cell suspensions or partitioned cells) may be subjected to conditions sufficient to denature one or more proteins. Denaturation may be achieved using heat, chemicals (e.g., SDS, urea, guanidine), reducing agents (e.g., dithiothreitol (DTT), beta mercaptoethanol, TCEP), urea, enzymes (e.g., ClpX, ClpS, unfoldases), ribozymes or DNAzymes. Similarly, the peptides or proteins may be subjected to conditions to solubilize the peptides or proteins in a solution, e.g., using detergents, organic solvents, spermidine, or tagging the peptides or proteins with polyionic tags (e.g., DNA, PEG, or other polymers). Alternatively, or in addition, the peptides or proteins may be enriched or purified; in an example, the peptides or proteins of interest may be precipitated using trichloroacetic acid, chloroform, trizole, or other chemical reagent. Other biological or chemical agents may be included during the protein processing, e.g., lysozymes, papain, cruzain, trypsin, protease inhibitors, nucleases or nuclease-containing proteins (e.g., DNAse, RNAse, DNA glycosylases, restriction endonucleases, transposases, micrococcal nucleases, Cas proteins).

Proteins and peptides may be processed such as to minimize disruption of protein-nucleic acid interactions. For instance, peptides or proteins may be crosslinked or fixed to DNA (e.g., using a chemical fixative such as PFA, glutaraldehyde, etc.). Crosslinking may occur within a cell or without a cell, e.g., in solution, in a partition such as a tube, microwell, etc. Accordingly, nucleic acid molecule-protein complexes (e.g., DNA or RNA bound to DNA-binding or RNA-binding proteins such as helicases, transcription factors, histones, polymerases, etc.) may be subjected to further processing and/or analysis, e.g., using the sequencing methods described herein. In some instances, DNA, RNA, protein/peptide sequence, or a combination thereof can be obtained concomitantly.

Peptides or proteins may be fragmented prior to analysis. Fragmenting proteins may be useful in reducing the size of the proteins and allow for efficient processing of peptides, as is described elsewhere herein. Fragmentation may be performed using proteases, e.g., trypsin, chymotrypsin, pepsin, Lys-C, Glu-C, Proteinase K, furin, thrombin, endopeptidase, papain, subtilisin, elastase, enterokinase, genenanse, endoproteinase, metalloproteases, or with chemical treatment, e.g., cyanogen bromide, hydrazine, hydroxylamine, formic acid, BNPS-skatole, iodosobenzoic acid, 2-nitro-5-thiocyanobenzoic acid, etc. Alternatively or in addition to, fragmentation may be performed using mechanical methods, such as sonication, vortexing, mechanical stirring, using temperature changes (e.g., freeze/thaw, heating), or other fragmentation approach.

Enrichment of proteins or peptides in a biological sample may be performed, e.g., for separating proteins and peptides from cellular debris or other types of analytes (e.g., nucleic acids, lipids, carbohydrates, metabolites). Such enrichment may include, for example, the use of affinity columns (e.g., ion exchange), size exclusion columns, affinity precipitation (e.g., immunoprecipitation), chemical precipitation (e.g., using trichloroacetic acid, chloroform, trizole), chromatography (e.g., HPLC), or electrophoresis. In instances where cells are partitioned prior to enrichment, the enrichment may be performed using microbeads, affinity microcolumns, affinity beads, etc. In some instances, fractionation may be performed on the proteins or peptides, which may be used to separate the proteins by size, hydrophobicity, charge, affinity, size, mass, density, etc.

Screening of a population of proteins or peptides in a biological sample may be performed. A population of peptides or proteins may be obtained or enriched from the biological sample (e.g., using affinity precipitation) based on a desired physical or chemical property. The enriched proteins may then be subjected to screening, e.g., to determine binding characteristics (e.g., affinity, selectivity, etc.) to a library of molecules. For example, the enriched proteins may be combined with a library of antigens or peptides to which a subpopulation of the enriched proteins may bind. The bound population or the unbound fraction may then be collected for further analysis and sequencing, using the methods described herein. Such a screening may be useful in diagnostic purposes, e.g., to determine if a biological sample (e.g., arising from a patient or subject) has been exposed to a particular antigen.

Peptides may be barcoded, in bulk or in partitions. Peptides may be barcoded with any useful type of barcode molecule, e.g., spectral or fluorescent barcodes, mass tags, nucleic acid barcode molecules, etc. The barcode molecules may allow for identification of an originating peptide, a partition, a sample, a cell, or cell compartment. For example, a cell sample may be partitioned such that a partition comprises at most one cell; the partition may comprise a unique barcode molecule (e.g., nucleic acid barcode molecule) that identifies the partition and thus the cell. Subsequent labeling of the peptides within the partition (e.g., by permeabilizing or lysing the cell) with the barcode molecules may be useful in identifying the peptides as arising or originating from the same cell or partition. In other examples, a substrate may comprise nucleic acid molecules comprising a unique barcode sequence that differs from barcode sequences of other substrates. As such, the barcode sequence may be used to identify the substrate. In some instances, barcoded substrates may be partitioned with cell samples, such that at least a subset of the partitions comprise a single cell and a single barcoded substrate. As such, the peptides arising from the single cell and transferred to the barcoded substrate may all be identifiable as originating from the single cell. Barcode molecules may comprise additional useful functional sequences, e.g., UMIs, primer sites, restriction sites, cleavage sites, transposition sites, sequencing sites, read sites, etc. Alternatively, or in addition to, a peptide or a plurality of peptides may be partitioned and barcoded in the partitions.

Attachment of barcode molecules to peptides may be achieved using any suitable chemistry. For example, C-terminal conjugation of nucleic acid barcode molecules may be achieved by amide coupling of amine-conjugated DNA barcode molecules to peptides or by thiol alkylation, e.g., reacting a thiolated peptide with an alkylated (e.g., iodoacetamide) DNA barcode molecule. N-terminal conjugation can be achieved, for instance, using 2-pyridinecarboxyaldehyde labeling of a DNA barcode and reacting with the N-terminus of a peptide. Internal residues, e.g., glutamate, can also be labeled with amine-conjugated DNA barcode molecules or carboxylated DNA barcodes (e.g., to react with primary amines in lysine).

Individual peptides may be barcoded at multiple locations for a given peptide. A peptide may be labeled at multiple sites with the same or different barcode sequences. For example, a peptide may be partitioned into a partition comprising a plurality of identical barcode molecules that comprise a barcode sequence that is unique to the partition. The peptide may be labeled at a single or multiple sites with the unique partition barcode sequence, optionally each comprising a unique molecular identifier (UMI), such that subsequent downstream analysis (e.g., sequencing) may be attributable to the same peptide using the barcode sequence. In some instances, a terminus of the peptide (e.g., N-terminus or C-terminus) or an internal amino acid may be labeled with a barcode. In some instances, the peptide may be fragmented prior to analysis or sequencing; accordingly, upstream attachment of multiple identical barcode molecules to the same peptide may allow for attribution of the sequence analysis back to a single peptide. Barcoding of peptides may occur prior to, during, or subsequent to fragmentation. Peptides may be labeled with barcodes (e.g., nucleic acid barcode molecules) using any suitable chemistry, e.g., as described above, or using bifunctional or trifunctional linkers comprising multiple linking moieties, e.g., as described elsewhere herein, such as click chemistry moieties, NHS-esters, EDC, native chemical ligation, etc. For example, C-terminal attachment may comprise amide coupling to C-terminus carboxylic group or photoredox tagging of C-terminus carboxylic group (e.g., to add an electrophile tag). N-terminal attachment may comprise amide coupling to N-terminus amine group, where specific attachment can occur at low pH, or using 2-pyridinecarboxaldehyde variants for specific attachment to N-terminus. Internal attachment may comprise, for example, amide coupling using EDC/NHS chemistry or DMT-MM to Glutamate or Aspartate; alkylation or disulfide bridge labeling of cysteines; or amide coupling to lysine residues.

In some examples, a peptide may be labeled with different barcode molecules, which can be indexed by proximity to one another, e.g., using primers that can anneal to adjacent barcode molecules. In one such approach, after a protein has been labeled with a plurality of barcodes with different barcode sequences, proximity-based polymerase extension may be used to copy and associate the sequence of adjacent barcodes. For example, each barcode molecule may comprise a primer binding site, to which a dual-primer linker sequence comprising two sequences is annealed. The dual primer linker sequence can bind to the primer binding sites of two adjacent barcodes. An extension reaction, e.g., using a polymerase, may extend and copy the barcode sequences of the adjacent barcodes. Subsequently, the dual primer linker sequence, which now has copies of the two adjacent barcodes, may be removed and sequenced. From the sequencing reads, an adjacency matrix of barcode sequences may be generated (e.g., to correspond barcode sequences on a single dual primer linker as spatially adjacent). Accordingly, each of the barcode sequences may be associated with a nearby adjacent barcode sequences, and as such, peptide portions may be aligned or attributed as being adjacent. Such an approach may be useful in instances where the peptide is fragmented, such that individual fragments of a peptide may be corresponded with the nearest neighbor using the barcode sequences.

In another example, a peptide may be barcoded at multiple locations for a given peptide using bridge amplification. In such an approach, a peptide or protein may be labeled at multiple sites with a nucleic acid primer. A nucleic acid barcode molecule may be provided, which can anneal to the nucleic acid primer (not shown) or be ligated to the nucleic acid primer. Subsequent rounds of bridge amplification may be performed in order to copy the nucleic acid barcode molecule to the other primers located at other sites of the given peptide. In some examples, a peptide may be tagged with multiple copies of the nucleic acid primer, and barcode sequences may be provided sparsely, such that only one nucleic acid primer per peptide is extended by polymerase extension. Subsequent rounds of bridge amplification can result in a peptide having the same barcode sequence at each nucleic acid primer. Subsequent fragmenting of peptides may be performed, such that peptide fragments comprise on average, a single barcode. Accordingly, in some cases, the output such an amplification approach may be peptides with individual barcodes generated from fragmenting multi-labeled proteins where peptides from the same protein have the same barcodes.

A sample of cells may be partitioned into individual partitions or compartments (e.g., droplets, microwells) such that at least a subset of the partitions comprise a single cell. The partitions may then be treated with a lysing agent to lyse the cells and release the proteins from the cells into the partition. The proteins may then be labeled with a partition-specific barcode (e.g., using a barcode bead), such that all peptides or proteins arising from a single compartment comprises the same barcode. In some examples, the barcodes comprise nucleic acid barcode molecules, and the barcode sequence can be used in downstream processing, e.g., via sequencing, the partition or cell from which a peptide originated. The nucleic acid barcode molecule may comprise any additional useful sequences, e.g., UMIs, primer sequences, etc.

Bulk Processing: A biological sample may be processed in bulk. For example, a biological sample may be processed to obtain a suspension of cells, which may be directly lysed in the suspension, without partitioning of cells in individual compartments. Cells may be lysed in bulk using any useful approach, e.g., as described above and optionally subjected to further processing, e.g., homogenization, protease inhibition, denaturation, protein processing (e.g., chemical treatment, fragmentation), or a combination thereof. A biological sample may be subjected to pre-processing prior to cell lysis or protein extraction. Such pre-processing may include removal of debris, purification, filtration, concentration, or sorting.

Additional conventional laboratory techniques and approaches may be utilized in the methods, systems, compositions, and kits of the present disclosure. Such conventional techniques and descriptions can be found in standard laboratory manuals such as: Cellular and Molecular Immunology, Ninth Edition, A. K. Abbas., et al., Elsevier (2017); Cancer Immunotherapy Principles and Practice, First Edition, L. H. Butterfield, et al., Demos Medical (2017); Janeway's Immunobiology, Ninth Edition, Kenneth Murphy, Garland Science (2016); Clinical Immunology and Serology: A Laboratory Perspective, Fourth Edition, C. Dorresteyn Stevens, et al., F. A. Davis Company (2016); Using Antibodies: A Laboratory Manual (Second Edition), Edward A. Greenfield, Cold Spring Harbor Laboratory Press (2015); Culture of Animal Cells: A Manual of Basic Technique and Specialized Applications, Seventh Edition, R. I. Freshney, Wiley-Blackwell (2016); Transgenic Animal Technology, Third Edition: A Laboratory Handbook, C. A. Pinkert, Elsevier (2014); The Laboratory Mouse, Second Edition, H. Hedrich, Academic Press (2012); Manipulating the Mouse Embryo: A Laboratory Manual, Fourth Edition, R. Behringer, et al., Cold Spring Harbor Laboratory Press (2013); PCR 2: A Practical Approach, M. J. McPherson, et al., IRL Press (1995); Methods in Molecular Biology (Series), J. M. Walker, ISSN 1064-3745, Humana Press; RNA: A Laboratory Manual, D. C. Rio, et al., Cold Spring Harbor Laboratory Press (2010); Methods in Enzymology (Series), Academic Press (2012); Molecular Cloning: A Laboratory Manual (Fourth Edition), M. R. Green, et al., Cold Spring Harbor Laboratory Press (2012); Bioconjugate Techniques, Third Edition, G. T. Hermanson, Academic Press (2013); Genome Analysis: A Laboratory Manual Series (Vols. I-IV), Cold Spring Harbor Laboratory Press (1998); Introduction to Laboratory Methods in Molecular, Cell, and Developmental Biology (Experimental Biology), C. Panaretos (2023); Introduction to Polymerase Chain Reaction (Experimental Biology), C. Panaretos (2023), Human Stem Cell Manual: A Laboratory Guide, 2nd Edition, S. Peterson and J. Loring Eds., Academic Press (2012); PCR Primer: A Laboratory Manual, C. W. Dieffenbach and G. S. Dveksler Eds., Cold Spring Harbor Press (2003); Lehninger, Principles of Biochemistry 3rd Ed., D. L. Nelson and M. M. Cox W. H. Freeman Pub., New York, N.Y. (2000); and Berg et al. Biochemistry, 5th Ed., W. H. Freeman Pub., New York, N.Y. (2002); all of which are herein incorporated in their entirety by reference for all purposes.

Spatial barcoding: A biological sample may comprise a tissue sample comprising multiple cells. Tissue samples may be processed using an approach to retain spatial information (e.g., to identify peptides from individual cells), e.g., using spatial barcodes. For instance, a 2-D or 3-D tissue sample may be provided, and individual cells or locations within a tissue sample may be contacted with a plurality of spatial barcodes (e.g., nucleic acid barcode molecules) comprising different barcode sequences. The different barcode sequences may be attributed to a particular location in the 2-D or 3-D tissue sample, which may correspond with a location of a cell. For example, spatial barcodes may be provided using deterministic methods such as two-photon patterning, or stochastic methods such as PCR, to assign different segments of the 2-D or 3-D tissue sample with unique spatial barcodes Accordingly, peptides that are labeled with spatial barcodes may be attributed back to a single location within a tissue sample, or back to a single cell.

In another example of a workflow of spatial barcoding of a tissue sample, the tissue sample comprising multiple cells (illustrated as a 2×2 array of cells) may be provided. The tissue sample may be subjected to lysis or fixation and permeabilization to provide access to the proteins contained within the multiple cells. Spatial barcodes, e.g., nucleic acid barcode molecules, may be provided. The spatial barcodes may comprise coordinate or location information. In an example, each cell may be contacted with a different spatial barcode, or portions of cells may be contacted with different spatial barcodes, which may optionally be pre-indexed (e.g., using imaging, or deterministic spatial barcoding approaches). Further processing of the peptides may be performed, as described elsewhere herein. As the peptides are labeled with the spatial barcodes, each peptide having a spatial barcode may be attributed back to its originating coordinate or location, which can help identify the originating cell from which a peptide arises.

In an example, a spatial barcode array may be provided on a substrate (e.g., a glass microscope slide, a hydrogel mesh). In some instances, the spatial barcodes may be directly conjugated to the substrate, or they may be provided on barcoded beads. In an example, a plurality of beads each comprising different barcode sequences may be arranged in an array on a substrate. Each bead may comprise a spatial barcode comprising a spatial barcode sequence, and optionally, a unique molecular identifier (UMI). A tissue sample (e.g., a fixed tissue sample) may be placed adjacent to (e.g., overlayed onto) the spatial barcode array. The tissue sample may then be subjected to conditions sufficient to transfer the peptides or proteins to the spatial barcode array. For example, the peptides or proteins may be transferred via passive transport, e.g., diffusion or Brownian motion, or via active transport, e.g., electrophoresis, pressure-driven flow, etc. The peptides or proteins may be attached to the spatial barcodes, e.g., using a linker (e.g., comprising amine-reactive groups, or click chemistry groups such as azide, alkyne, or other functional moieties such as aldehyde groups or NHS or carboxylic groups), conjugation chemistry, or an anchoring agent. Examples of anchoring agents include fixatives, such as formaldehyde, paraformaldehyde, glutaraldehyde, or monomers for incorporation into a hydrogel, e.g., Acryloyl-X, acrylamide, N-(3-Aminopropyl) methacrylamide, or N-(3-Aminoethyl) methacrylamide, or benzophenone. Anchoring agents may also comprise multi-functional linkers, e.g., Acryloyl-X, Biotin-NHS, Biotin-PEG-Amine, DBCO-NHS, DBCO-amine. For bead arrays, the plurality of beads may be collected from the sample for further processing.

Additional examples of methods, systems, and compositions of processing samples and peptides are provided in International Patent Pub. No. WO 2023/114732, which is incorporated by reference herein in its entirety.

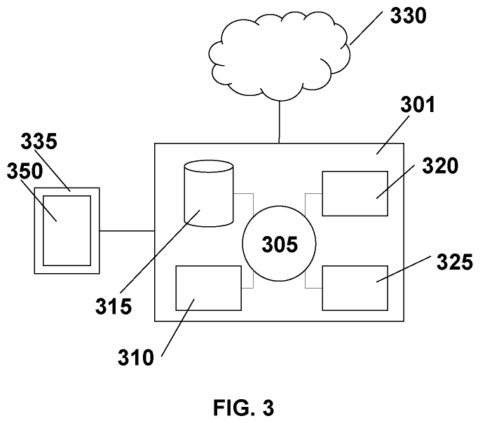

Computer Systems and Computer-Implemented Methods. The present disclosure provides computer systems that are programmed to implement methods of the disclosure. FIG. 3 shows a computer system 301 that is programmed or otherwise configured to sequence or process sequencing reads of a polymeric analyte. The computer system 301 can regulate various aspects of polymeric analyte sequencing of the present disclosure, such as, for example, processing sequencing reads and outputting amino acid identity. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 350 for providing, for example, data on sequencing runs and sequencing data. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, process sequencing reads and output data regarding the sequencing reads, including without limitation run parameters, sequencing read count, identities of polymeric analytes, statistics of the polymeric analytes, etc.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

As used herein, the term “protein” generally refers to a molecule comprising two or more amino acids joined by a peptide bond. A protein may also be referred to as a “polypeptide”, “oligopeptide”, or “peptide”. A protein can be a naturally occurring molecule, or a synthetic molecule (e.g., an artificial protein, peptide, enzyme). A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications or by chemical modification. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “peptide” may refer to any short, single peptide chain. A peptide may be no more than about 100, 95, 90, 85, 80, 75, 70, 65, 60, 55, 50, 45, 40, 35, 30, 25, 20, 15, 10, 5, or less than about 5 amino acids in length. A peptide may have a known or unknown biological function or activity. Peptides can include natural, synthetic, modified, or degraded proteins or peptides, or a combination thereof. Peptides can include proteinogenic, natural, synthetic, or modified amino acids or amino acid residues, or a combination thereof.

As used herein, the term “single analyte” may refer to an analyte that is individually manipulated or distinguished from other analytes. A single analyte may comprise a biomolecule or a synthetic molecule. A single analyte may comprise a small molecule. A single analyte can be a single molecule (e.g., a single biomolecule such as a single protein, nucleic acid molecule, affinity reagent, lipid, carbohydrate, metabolite, hapten, small molecule, pharmaceutical compound, nanoparticle, amino acid derivative, synthetic amino acid, etc.), a single complex of two or more molecules (e.g., a multimeric protein having two or more separable subunits, a single protein attached to a nucleic acid molecule or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, “polypeptide” refers to two or more amino acids linked together by a peptide bond. The term “polypeptide” includes proteins that have a C-terminal end and an N-terminal end as generally known in the art and may be synthetic in origin or naturally occurring. As used herein “at least a portion of the polypeptide” refers to 2 or more amino acids of the polypeptide. A polypeptide may comprise one or more peptides. Optionally, a portion of the polypeptide includes at least: 1, 5, 10, 20, 30 or 50 amino acids, either consecutive or with gaps, of the complete amino acid sequence of the polypeptide, or the full amino acid sequence of the polypeptide.

As used herein, “affixed” refers to a connection between two molecules that are held in physical proximity. The term “affixed” encompasses both an indirect or direct connection and may be reversible or irreversible, for example the connection is optionally a covalent bond or a non-covalent bond.

As used herein, the term “sample” refers to a collected substance or material that comprises or is suspected to comprise one or more analytes of interest (e.g., biomolecules, e.g., polypeptides). A sample may be modified for purposes such as storage or stability. A sample may be naturally occurring or synthetic. A sample may be processed to separate or remove unwanted fractions or impurities from the analyte(s) of interest. A sample may be enriched or purified. For example, a sample may comprise a fraction of a separation process (e.g., chromatography, fractionation, electrophoresis, etc.). Alternatively, a sample may not be subjected to processing that separates or removes any unwanted fractions or impurities from the analyte(s) of interest. A sample may be obtained from any suitable source or location, including from organisms, cells, tissues, cell preparations, cell-free compositions, the environment (e.g., air, water, dirt, soil, agriculture, soil, dust, sewage). A sample may be obtained from an organism or part of an organism, such as from a fluid, tissue, or cell. A sample may include biological and/or non-biological components. As used herein, the terms “biological sample” or “biological source” refer to a sample that is derived from a predominantly biological system or organism, such as one or more viral particles, cells (e.g. individualized cells), organelles (e.g. individualized organelles), tissues, organs, bodily fluids, bone, cartilage, and exoskeleton. A biological sample may comprise a prokaryotic cell (e.g., bacteria) or eukaryotic cell (e.g., fungus, protist, algae, plant, animal). A biological sample may comprise a majority of biological material on a mass basis, excluding the weight of fluid within the sample. Biological samples may comprise one or more proteins, referred to herein as protein samples. Biological samples can be acquired from various sources, e.g., from a clinical patient sample, such as blood, serum, plasma, Cerebral Spinal Fluid (CSF), saliva, mucosal secretions, sputum, urine, lymph, perspiration, vaginal fluid, semen, fecal matter, amniotic fluid, perspiration, synovial fluid, fine needle aspirates, a tissue biopsy, a tumor biopsy, etc. A biological sample may be processed to purify and retain one or more biomolecules (e.g., proteins, nucleic acids, carbohydrates, lipids, glycoproteins, lipoproteins, metabolites, etc.) from the biological sample. A biological sample (e.g., a protein sample) may be derived from cultured cells, which may be treated or untreated. A biological sample (e.g., a protein sample) can also result from tissue specimens, such as biopsy samples, which may optionally be processed to liberate biomolecules (e.g., proteins) contained therein. Tissue samples may also be derived from in vivo specimens, including fresh, frozen, acute, and fixed tissues. A sample or a biological sample may comprise non-biological molecules, including but not limited to nanoparticles, polymers, haptens, small molecules, chemicals, fluorescent reagents, inert materials, pharmaceuticals, food additives, environmental contaminants, solvents, industrial chemicals, nanomaterials, radioisotopes, by-products from non-biological molecules.

As used herein, the term “hydrogel” refers to a three-dimensional polymeric structure that is substantially insoluble in water, but which is capable of absorbing and retaining large quantities of water to form a substantially stable, often soft and pliable, structure. In some embodiments, water can penetrate in between polymer chains of a polymer network, subsequently causing swelling and the formation of a hydrogel. In some embodiments, hydrogels are super-absorbent (e.g., containing more than about 90% water) and can be included of natural or synthetic polymers. Examples of hydrogels include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G) A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. A detailed description of suitable hydrogels may be found in published U.S. Patent Publication US 2010/0055733, herein specifically incorporated by reference. As used herein, the terms “hydrogel subunits” or “hydrogel precursors” mean hydrophilic monomers, prepolymers, or polymers that can be crosslinked, or “polymerized”, to form a three-dimensional (3D) hydrogel network. It is believed that this fixation of the biological specimen in the presence of hydrogel subunits crosslinks the components of the specimen to the hydrogel subunits, thereby securing molecular components in place, preserving the tissue architecture and cell morphology.

As used herein, the terms “antibody” and “immunoglobulin” may generally refer to proteins that can recognize and bind to a specific antigen. An antibody or immunoglobulin may refer to an antibody isotype, fragments of antibodies including, but not limited to, Fab, Fv, scFv, vHH, and Fd fragments, chimeric antibodies, humanized antibodies, single-chain antibodies, and fusion proteins including an antigen-binding portion of an antibody and a non-antibody protein. The antibodies may be detectably labeled, e.g., with a fluorophore, radioisotope, enzyme (e.g., a peroxidase), epitope tag, which generates a detectable product, fluorescent protein, nucleic acid barcode sequence, and the like. The antibodies may be further conjugated to other moieties, such as members of specific binding pairs, e.g., biotin (member of biotin-avidin specific binding pair), and the like. Also encompassed by the terms are nanobodies, Fab′, Fv, F(ab′)2, scFv, and other antibody fragments that retain specific binding to antigen. Antibodies may exist in a variety of other forms including, for example, Fv, Fab, and (Fab)2, diabodies, monobodies, single domain antibodies (sdAb), as well as bi-functional (i.e., bi-specific, e.g., bi-specific T-cell engager) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N.Y., 2nd ed. (1984), and Hunkapiller and Hood, Nature, 323, 15-16 (1986), which are herein incorporated by reference). Naturally occurring immunoglobulins or antibody types include immunoglobulin A, immunoglobulin G, immunoglobulin D, immunoglobulin E, immunoglobulin M, or other immunoreactive components.

“Binding” or “coupling” as used herein generally refers to a covalent or non-covalent interaction between two molecules (referred to herein as “binding partners”, e.g., a substrate and an enzyme or an antibody and an epitope). Binding between binding partners may be specific or non-specific. Binding between binding partners may involve one or more additional molecules (e.g., biomolecules) or enhancer molecules or substrates.

As used herein, “specifically binds” or “binds specifically” generally refers to an interaction between binding partners (e.g., a binding partner and a cognate molecule) such that the binding partners bind to one another, but do not bind to other molecules that may be present in the environment (e.g., in a biological sample, in tissue, in an in vitro assay) under a set of conditions. A specific binding interaction may entail a binding partner that binds to a cognate molecule. The specific binding interaction may entail the binding of the binding partner to its cognate molecule at a significantly or substantially higher level or with greater affinity as compared to the binding of the binding partner to a non-cognate molecule. A specific binding interaction may entail a first binding partner that has greater selectivity of binding to the cognate molecule as compared to a non-cognate molecule.

The terms “nucleic acid”, “nucleic acid molecule”, “oligonucleotide” and “polynucleotide” may be used interchangeably herein and generally refer to a polymeric form of naturally occurring or synthetic nucleotides, or analogs thereof, of any length. A nucleic acid molecule may comprise one or more deoxyribonucleotides, deoxynucleotide triphosphates, dideoxynucleotide triphosphates, deoxynucleotide hexaphosphates, dideoxynucleotide hexaphosphates, ribonucleotides, hexitol nucleotides, cyclohexane nucleotides, or analogs or combinations thereof. A nucleic acid molecule may comprise, e.g., DNA, RNA, HNA, CeNA, and modified forms thereof. A nucleic acid molecule may comprise nucleotides that are linked by phosphodiester bonds. A nucleic acid molecule may have any two- or three-dimensional structure, and may perform any function, known or unknown. A nucleic acid molecule may be single stranded, double stranded, or partially double stranded. A nucleic acid molecule may be single stranded orr double stranded. Non-limiting examples of polynucleotides include a gene, a gene fragment, exons, introns, messenger RNA (mRNA), transfer RNA, ribosomal RNA, noncoding RNA, small interfering RNA, short hairpin RNA, micro RNA, scaRNA, ribozymes, riboswitches, viral RNA, complementary DNA (cDNA), cosmid DNA, mitochondrial DNA, chromosomal or genomic DNA, viral DNA, recombinant polynucleotides, branched polynucleotides, plasmids, vectors, isolated DNA of any sequence, control regions, isolated RNA of any sequence, nucleic acid probes, nucleic acid adapters, and primers. The nucleic acid molecule may be linear, circular, or any other geometry. Examples of polynucleotide analogs include but are not limited to xeno nucleic acid (XNA), bridged nucleic acid (BNA), glycol nucleic acid (GNA), hexitol nucleic acid (HNA), cyclohexane nucleic acid (CeNA), 2′-F-Arabinonucleic acids (2′-F-ANA), peptide nucleic acids (PNAs), γPNAs, morpholino polynucleotides, locked nucleic acids (LNAs), non-constrained nucleic acid (NNA), threose nucleic acid (TNA), D-threoninol nucleic acid (D-aTNA), L-threoninol nucleic acid (L-aTNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, inverted base, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In addition, a nucleic acid molecule may exist permanently or transitionally in single-stranded or double-stranded form, including homoduplex, heteroduplex, and hybrid states.

As used herein, the term “amino acid” generally refers to an organic compound that combines to form a protein or peptide. An amino acid generally comprises an amine group, a carboxylic acid group, and a side-chain specific to each amino acid, which serve as a monomeric subunit of a peptide. An amino acid may include the 20 standard, naturally occurring or canonical amino acids as well as non-standard or non-canonical amino acids. The standard, naturally-occurring or canonical amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, and N-methyl amino acids.

As used herein, the term “amino acid type” generally refers to one of the standard, naturally-occurring or canonical amino acids, e.g., one member of the group consisting of Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), Tyrosine (Y or Tyr), derivatives thereof, and modified forms of any of the aforementioned amino acids. The term “amino acid type” may be used herein to distinguish a plurality of amino acids that comprise different side chain groups, rather than a plurality of amino acids that are identical (e.g., different positional amino acids of a single peptide that have the same side chain). An amino acid type may comprise a modified version of one of the standard, naturally-occurring or canonical amino acids e.g., post translational modifications, an epigenetic modification, or chemical or enzymatic modifications. In some instances, an amino acid type can include non-canonical amino acids.

As used herein, the term “post-translational modification” refers to modifications that occur on a peptide subsequent to translation. A post-translational modification may be a covalent modification or enzymatic modification. Examples of post-translation modifications include, but are not limited to, acylation, acetylation, alkylation (including methylation), benzoylation, biotinylation, butyrylation, carbamylation, carbonylation, carboxylation, crotonylation, deamidation, deiminiation, dimethylation, diphthamide formation, disulfide bridge formation, eliminylation, flavin attachment, formylation, gamma-carboxylation, glutamylation, glutarylation, glycylation, glycosylation, glypiation, heme C attachment, hydroxylation, hypusine formation, iodination, isoprenylation, lipidation, lipoylation, malonylation, methylation, myristolylation, nitration, oxidation, transglutamination, palmitoylation, pegylation, phosphopantetheinylation, phosphorylation, prenylation, propionylation, pyroglutamate formation, retinylidene Schiff base formation, S-glutathionylation, S-nitrosylation, S-sulfenylation, S-adenosylation, sulfation, selenation, stearoylation, succinylation, sulfination, trimethylation, ubiquitination, and C-terminal amidation. A post-translational modification includes modifications of the amino terminus and/or the carboxyl terminus of a peptide. Modifications (both naturally occurring and synthetic) of the terminal amino group include, but are not limited to, des-amino, N-lower alkyl, N-di-lower alkyl, and N-acyl modifications, N-terminal cyclization, deamination, oxidation, ubiquitination, SUMOylation, Neddylation, ISGylation, pupylation, eliminylation, biotinylation, lipidation, N-terminal methylation, N-terminal acetylation, N-terminal propionylation, N-terminal butyrylation, N-terminal crotonylation, N-terminal myristoylation, N-terminal palmitoylation, N-terminal stearoylation, and N-terminal benzoylation. Modifications of the terminal carboxy group include, but are not limited to, amide, lower alkyl amide, dialkyl amide, and lower alkyl ester modifications (e.g., wherein lower alkyl is C1-C4 alkyl). A post-translational modification also includes modifications, such as but not limited to those described above, of amino acids falling between the amino and carboxy termini. The term post-translational modification can also include peptide modifications that include one or more detectable labels. A post-translational modification may be naturally occurring or synthetic.

As used herein, the term “binding agent” refers to a molecule, e.g., a nucleic acid molecule, a peptide, a polypeptide, a protein, carbohydrate, a synthetic molecule, or a small molecule that binds to, associates with, unites with, recognizes, or combines with another molecule. The binding agent may bind to a macromolecule or a component or feature of a macromolecule. A binding agent may form a covalent association or non-covalent association with a molecule, a macromolecule, or a component or feature of a macromolecule. A binding agent may also be a chimeric binding agent, composed of two or more types of molecules, such as a nucleic acid molecule-peptide chimeric binding agent, a carbohydrate-peptide chimeric binding agent, or a lipid-peptide chimeric binding agent. A binding agent may be a naturally occurring, synthetically produced, or recombinantly expressed molecule. A binding agent may bind to a single monomer or subunit of a polymeric analyte, such as a macromolecule (e.g., a single amino acid of a peptide) or bind to a plurality of linked subunits of a macromolecule (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid over a non-modified or unlabeled amino acid. For example, a binding agent may preferably bind to an amino acid that has been modified with an acetyl moiety, guanyl moiety, dansyl moiety, PTC moiety, DNP moiety, SNP moiety, etc., over an amino acid that does not possess such a moiety. A binding agent may bind to a post-translational modification, either naturally occurring or synthetic, of a peptide molecule. A binding agent may exhibit selective binding to a component or feature of a macromolecule (e.g., a binding agent may selectively bind to one of the 20 possible natural amino acid residues and bind with very low affinity or not at all to the other 19 natural amino acid residues). A binding agent may exhibit less selective binding, where the binding agent is capable of binding a plurality of components or features of a macromolecule (e.g., a binding agent may bind with similar affinity to two or more different amino acid residues). A binding agent may comprise a tag, which may be coupled to the binding agent via a linker.

As used herein, the term “linker” generally refers to a molecule or moiety that is involved in joining two or more molecules. A linker may facilitate a covalent or noncovalent interaction of two or more molecules. A linker may be a crosslinker. The linker can be unifunctional, bifunctional, trifunctional, quadrifunctional, or polyfunctional. A linker can be chiral or achiral, or may contain one or more chiral centers, to influence enzymatic or chemical reactivity, conformational dynamics, steric interactions, or electronic properties of the linked molecules. A linker can be or comprise a nucleotide, a nucleotide analog, an amino acid, a peptide, a polypeptide, or a non-nucleotide chemical moiety, such as an organic or inorganic compound. A linker may comprise a polymer, such as a polyethylene glycol (PEG), polyethylene (PE), polypropylene (PP), polyvinyl chloride (PVC), polystyrene (PS) poly-L-lysine (PLL), poly(DL-lactic acid) (PLA), poly(DL-lactide-co-glycoside) (PLGA), polyornithine, polyarginine, or other organic or inorganic polymer. A linker may comprise one or more reactive ends, e.g., an amine-reactive group, a carboxyl-reactive group, a sulfhydryl-reactive group, a hydroxyl-reactive group, etc. Alternatively, a linker may not comprise a reactive end. In some examples, a linker may be used to join different molecule types, e.g., different biomolecule types such as a peptide with a nucleic acid molecule, a lipid with a peptide, a carbohydrate with a peptide, etc.; non-biomolecule types; or a biomolecule to a non-biomolecule. For example, a linker may be used to join a binding agent with a tag, a tag with a macromolecule (e.g., peptide, nucleic acid molecule), a macromolecule with a solid support, a tag with a solid support, etc. A linker may join two molecules via enzymatic reaction or chemistry reaction (e.g., click chemistry). A linker may join more than two molecules, e.g., via enzymatic or chemical reactions. A linker may influence reaction kinetics, yield, or product specificity by modulating molecular proximity, steric hindrance, or electronic environment. For example, a linker may enhance or inhibit reaction rates by controlling the spatial arrangement of reactants, stabilizing intermediates, or facilitating catalytic interactions. In some reactions, a linker may stabilize or position reaction intermediates in a favorable orientation, thereby influencing reaction efficiency, pathway selection, or product distribution. In chemical synthesis, a linker may dictate regioselectivity or stereoselectivity by constraining molecular conformation. Additionally, a linker may participate in dynamic structural changes, enabling conformational flexibility or rigidity to promote or suppress specific reaction pathways. A linker may modulate product stability, for example, by reducing susceptibility to hydrolysis or degradation. In some cases, a linker may introduce functional groups that participate in subsequent transformations, thereby influencing multi-step reaction cascades. A linker can be relatively linear or non-linear, e.g., cyclic or circularized, branched, polygonal, etc.

The term “conjugated” as used herein generally refers to a covalent or ionic interaction between two entities, e.g., molecules, compounds, or combinations thereof.

As used herein, the term “tag” generally refers to a molecule or moiety that is conjugated to a molecule. A tag may comprise a detectable label, e.g., a fluorophore or fluorescent protein, a radioactive isotope, an enzyme (e.g., a chromogenic or fluorescent protein, proteins that can catalyze chromogenic substrates), a mass tag, a hapten (e.g., biotin, digoxigenin, urushiol, fluorescein), a vibrational or FTIR tag (e.g., alkyne group). A tag may comprise a biomolecule, such as a nucleic acid molecule, a protein, a lipid, a carbohydrate, or a combination thereof. A tag may comprise one or more nucleic acid molecules, which may optionally encode information regarding the tag or the molecule onto which a tag is conjugated (e.g., a binding agent, such as an antibody). For example, a tag may comprise a nucleic acid barcode molecule. A tag may comprise an organic compound or an inorganic compound. As used herein, the term “tag” may also refer to a patterned sequence of signals, wherein signals appear, skip, or disappear at defined intervals, creating a recognizable marker or reference within the signal data. This patterned appearance may encompass periodic or non-periodic intervals between signals, selective omissions, or complex structured combinations of signal presence and absence that collectively form a unique, detectable signature. For example, a tag may comprise a nucleic acid barcode molecule that has a series of distinct vibrational signatures detectable by techniques such as Raman or FTIR spectroscopy.

As used herein, the term “barcode” generally refers to an identifying feature that may be used to distinguish similar items. A barcode may comprise a nucleic acid molecule of about 2 to about 150 bases. A barcode may comprise a nucleic acid molecule of about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150 or more bases, which may provide a unique identifier tag or origin information for a molecule (e.g., protein, polypeptide, peptide), a binding agent, a set of binding agents from a binding cycle, a sample molecule, a set of samples, molecules within a compartment (e.g., droplet, bead, partition or separated location), macromolecules within a set of compartments, a fraction of macromolecules, a set of macromolecule fractions, a spatial region or set of spatial regions, a library of macromolecules, or a library of binding agents. A barcode can be an artificial sequence or a naturally occurring sequence including peptides, proteins, protein complexes, carbohydrates, and synthetic polymeric materials such as peptoids, polysaccharides, polymers, fluorescent tags, chemical tags, magnetic tags, isobaric tags, Raman spectroscopic tags, quantum dots, etc. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. A population of barcodes may comprise error correcting barcodes. Barcodes can be used to computationally deconvolute sequence reads derived from an individual molecule, partition, sample, library, etc. Barcodes may comprise multiplexed information, e.g., arising from different samples, compartments, individual molecules, etc. A barcode can also be used for deconvolution of a collection of molecules that have been distributed into small compartments for enhanced mapping. For example, rather than mapping a peptide back to the proteome, the peptide can be mapped back to its originating protein molecule or protein complex, a sample or partition from which it originated, etc. A barcode may comprise any useful structure moiety or motif, e.g., hairpins, loop sequences, or spacers. Barcodes can comprise artificial or modified nucleic acids, e.g., locked nucleic acids (LNA), protein nucleic acids (PNA), hexitol nucleic acids (HNA), cyclohexane nucleic acids (CeNA), or a combination thereof. Barcodes may comprise or be generated using a protein, e.g., Tal effector, Cas protein (e.g., Cas9), Argonaut, or coiled coils. A barcode may comprise any useful sequence, including repeat sequences (e.g., a poly-A, poly-T, poly-C, poly-G region) or the barcode may comprise non-repeat sequences. A barcode may encode for information including, but not limited to time, lineage, sample types, cell number, beads, single molecule information, meta data, space/location (e.g., a slide, well, tissue), proximity (e.g., to other molecules, cells, metabolites, DNA, RNA), patient info, biological sample information, library information computer data, weather, physical parameters such as temperature, humidity, precipitation.

As used herein, a “sample barcode”, also referred to as “sample tag” generally refers to a barcode molecule comprising identifying information of a sample from which a barcoded molecule derives.

As used herein, a “spatial barcode” generally refers to a barcode molecule comprising identifying information of a region of a 2-D or 3-D sample (e.g., a tissue section) from which a molecule originates or is derived. Spatial barcodes may be used for molecular pathology on tissue sections. A spatial barcode may allow for multiplex sequencing of a plurality of samples or libraries from tissue section(s).

As used herein, a “temporal barcode” generally refers to a barcode molecule comprising time-based information relating to the barcoded molecule. The types of time-based data encoded in a temporal barcode can include information such as a lifetime of a barcoded molecule, a time of collection of a sample, a time or duration since the beginning of an experiment or induction with a stimulus, information on the age of a cell or tissue, a sequence of interactions between molecules, a time or cycle or round (e.g., of an iterative process) in which the barcode molecule is provided, among others. It is possible for different types of barcodes (e.g., spatial, temporal, cell-specific) to be combined in one multiplexed barcode.

As used herein, the term “fluorescent label,” “fluorescent tag,” or “fluorophore” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer. Fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes. Examples of fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For exemplary methods for custom synthesis of nucleotides having other fluorophores, see, Henegariu et al. (2000) Nature Biotechnol. 18:345. More examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. All references are herein incorporated by reference in their entireties.

As used herein, the term “nucleic acid sequence” or “oligonucleotide sequence” generally refers to a contiguous string of nucleotide bases and may refer to the particular placement of nucleotide bases in relation to each other as they appear in an oligonucleotide. Similarly, the term “polypeptide sequence,” “peptide sequence,” or “amino acid sequence” refers to a contiguous string of amino acids and may refer to the particular placement of amino acids in relation to each other as they appear in a polypeptide.

The terms “complementary” or “complementarity” refer to polynucleotides (i.e., a sequence of nucleotides) related by base-pairing rules. For example, the sequence “5′-AGT-3′,” is complementary to the sequence “5′-ACT-3”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the melting temperature of the formed hybrid. Hybridization methods involve the annealing of one nucleic acid to another, complementary nucleic acid, e.g., based on Watson-Crick base pairing.

As used herein, the term “proteomics” generally refers to quantitative and/or qualitative analysis of the proteome within a sample, such as biological sample, e.g., from cells, tissues, or bodily fluids. Proteomics may include the analysis of spatial distributions of proteins within a sample (e.g., cell and/or tissues). Proteomics may include studies of the dynamic state of the proteome, e.g., how one or more proteins change in time. A proteome may comprise multiple “-omes”, e.g., a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition (e.g., cancer, disease); a proteome subset associated a cellular process, such as cell cycle, differentiation (or de-differentiation), cell death, senescence, cell migration, transformation, or metastasis; or any combination thereof.

The terminal amino acid at one end of the peptide chain that has a free amino group may be referred to herein as the “N-terminal amino acid” (NTAA). The terminal amino acid at the other end of the chain that has a free carboxyl group may be referred to herein as the “C-terminal amino acid” (CTAA). The amino acids making up a peptide may be numbered in order, with the peptide being “n” amino acids in length. As used herein, in some instances, NTAA may be considered the nth amino acid (also referred to herein as the “n NTAA”). In such cases, the next amino acid is the n-1 amino acid, then the n-2 amino acid, and so on down the length of the peptide from the N-terminal end to C-terminal end. Alternatively, CTAA may be considered the nth amino acid (also referred to herein as the “n CTAA”). In such cases, the next amino acid is the n-1, then the n-2 amino acid, and so on down the length of the peptide from the C-terminal end to N-terminal end. An NTAA, CTAA, or both may be modified or labeled with a chemical moiety.

As used herein, the terms “determining,” “measuring,” “assessing,” and “assaying” are used interchangeably and include both quantitative and qualitative determinations.

As used herein, the term “unique molecular identifier” or “UMI” generally refers to a molecule barcode comprising indexing information. A UMI may comprise a nucleic acid molecule of about 3 to about 150 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases) in length. A UMI may provide a unique identifier tag for each molecule (e.g., peptide, binding agent, a nucleic acid molecule) that comprises or is coupled to a UMI. A UMI may comprise a random sequence (e.g., a random N-mer).

As used herein, a “derivative” with reference to a nucleic acid molecule generally refers to a nucleic acid molecule that is derived from an originating nucleic acid molecule. The derivative may have the same or substantially the same nucleotide sequence as the originating nucleic acid molecule, or the derivative may comprise a complement or partial complement as the originating nucleic acid molecule. A derivative may be the same type of nucleic acid (e.g., DNA or RNA) as the originating nucleic acid molecule, or the derivative may be a different type of nucleic acid (e.g., cDNA generated from an RNA molecule). A nucleic acid molecule derivative may display sequence identity as the originating nucleic acid molecule. The derivative nucleic acid molecule may also be subjected to additional processing from the originating nucleic acid molecule, e.g., chemical or enzymatic modification, splicing, ligation, polymerization, fragmentation, tagmentation (e.g., using a transposase), digestion, etc.

A “derivative polypeptide” or “derivative peptide” as used herein is a polypeptide or peptide derived from an originating polypeptide (or peptide). A derivative may comprise the same amino acid sequence as the originating polypeptide, or the sequence may be different. The derivative polypeptide may result from or be subjected to additional processing from the originating polypeptide, e.g., chemical or enzymatic modification. The derivative polypeptide may comprise one or more tags, nucleic acid molecules, barcode molecules, labels (e.g., detectable labels), fluorophores, probes, linkers, post-translational modifications, chemical protecting groups, or other chemical moieties.

A “derivative” with reference to a modified amino acid disclosed herein may generally refer to a molecule that is derived from the modified amino acid. A derivative may be a product of a reaction (e.g., chemical, enzymatic) or interaction of the modified amino acid with another molecule. Further processing of the modified amino acid may optionally be performed to arrive at the “derivative thereof.” For example, further chemical or enzymatic treatment, cleavage of the amino acid, an extension or amplification reaction, cleavage or removal from a substrate, physical processes such as mechanical shearing or fragmentation may be performed on the modified amino acid to obtain a derivative of the amino acid or derivative of the modified amino acid. In an example, a modified amino acid may be derivatized by chemical reaction, e.g., using maleimide to react with cysteine residues, NHS esters or isothiocyanates to react with lysine residues, etc. The derivative may result from performing a nucleic acid reaction (e.g., nucleic acid extension reaction, amplification, ligation, transposition, hybridization, dehybridization, etc.). In some instances, a modified amino acid may refer to a stacked plurality of modified amino acids, as described elsewhere herein.

As used herein, the term “compartment” or “partition” generally refers to a physical area or volume that separates or isolates a subset of molecules from a sample of molecules. For example, a compartment or partition may separate an individual cell from other cells, or a subset of a sample's proteome from the rest of the sample's proteome. A compartment or partition may be an aqueous compartment (e.g., microfluidic droplet), a solid compartment (e.g., picotiter well or microtiter well on a plate, tube, vial, gel bead), a liquid-liquid phase separation, a liquid condensate, a subcellular region, or a separated region on a surface. A compartment may comprise one or more beads to which macromolecules may be immobilized. A compartment may be transient.

As used herein, the term “carrier fluid” refers to a fluid configured or selected to contain one or more discrete entities, e.g., droplets, as described herein. A carrier fluid may include one or more substances and may have one or more properties, e.g., viscosity, which allow it to be flowed through a microfluidic device or a portion thereof, such as a delivery orifice. In some embodiments, carrier fluids include, for example: oil or water, and may be in a liquid or gas phase. Suitable carrier fluids are described in greater detail herein.

As used herein, the term “solid support”, “solid surface”, or “solid substrate” or “substrate” refers to any solid material, including porous and non-porous materials, to which a molecule can be associated directly or indirectly. The molecule may be associated with the substrate by covalent or non-covalent interactions, or a combination thereof. A substrate may be two-dimensional (e.g., planar surface) or three-dimensional (e.g., gel matrix or bead). A solid support may comprise, in non-limiting examples, a bead, a microbead, an array, a glass surface, a silicon surface, a plastic surface, a filter, a membrane, nylon or other polymer, a silicon wafer chip, a flow through chip, a flow cell, a microfluidic device or chip or a surface thereof, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, (e.g., nanotubes), particles, beads, DNA origami, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a magnetic or paramagnetic bead, a glass bead, or a controlled pore bead, or a combination thereof. A magnetic bead may comprise or be composed of any useful material and may respond to an applied magnetic field. A bead may be spherical or an irregularly shaped. A bead's size may range from nanometers, e.g., 1 nm, 10 nm, 100 nm, to millimeters, e.g., 1 mm. In certain embodiments, beads range in size from about 0.2 micron to about 200 microns, or from about 0.5 micron to about 5 microns. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μm in diameter. In certain embodiments, “a bead” solid support may refer to an individual bead or a plurality of beads. A solid support may assume any useful geometry, e.g., pyramid, cube, cylinder, helix, sphere, spheroid, rod, disc, arrow, spring, teardrop, prism, tetrapod, or any other useful geometry. A bead may be coated or treated with a range of substances or surface modifications to alter its physical, chemical, or biological properties.

As used herein, “sequencing” generally refers to determining the order and identity of: (A) nucleotides (base sequences) in a nucleic acid sample, e.g., DNA or RNA; (B) amino acids in all or part of a polymer, such as a protein, peptide, or other multimeric molecule; or (C) monomers in all or part of a polymer (e.g., a synthetic or naturally occurring polymer, e.g., with more than one type of monomer). Many techniques are available for nucleic acid sequencing, such as Sanger sequencing or High Throughput Sequencing technologies (HTS). Sanger sequencing may involve sequencing via detection through (capillary) electrophoresis, in which up to 384 capillaries may be sequence analyzed in one run. High throughput sequencing involves the parallel sequencing of thousands or millions or more sequences at once. HTS can be defined as Next Generation sequencing (NGS), i.e. techniques based on solid phase pyrosequencing or as Next-Next Generation sequencing based on single nucleotide real time sequencing (SMRT). HTS technologies are available such as offered by Roche, Illumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Additional sequencing methods include Raman sequencing and Infrared (IR) sequencing, which utilizes Raman spectroscopy or IR spectroscopy to detect molecular vibrations associated with specific nucleotide or amino acid sequences, enabling label-free sequencing based on unique vibrational energy signatures. Tunneling current sequencing identifies base sequences or amino acid sequences through electronic signal variations as nucleotides or amino acids pass through a nanoscale gap, detecting characteristic tunneling currents specific to each molecular component.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, nanopore sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, ThermoFisher, and Roche, including formats such as parallel bead arrays, sequencing by synthesis, sequencing by ligation, capillary electrophoresis, electronic microchips, “biochips,” microarrays, parallel microchips, and single-molecule arrays, zero mode waveguide based sequencing, some of which are reviewed by Service (Science 311:1544-1546, 2006).

As used herein, “analyzing” means to quantify, characterize, identify, distinguish, or a combination thereof, all or a portion of the components of a molecule (e.g., a macromolecule, a biological molecule such as a protein, amino acid, nucleic acid molecule, etc.). For example, analyzing a peptide, polypeptide, or protein may comprise determining all or a portion of the amino acid sequence (contiguous or non-continuous) of the peptide. Analyzing a macromolecule may include partial identification of a component of the macromolecule. For example, partial identification of amino acids in a protein sequence can identify an amino acid in the protein as belonging to a subset of possible amino acids. Analysis may be performed sequentially, e.g., beginning with analysis of the n NTAA, and then proceeding to the next amino acid of the peptide (i.e., n-1, n-2, n-3, and so forth). In such instances, sequencing may be performed by cleavage of the n NTAA, thereby converting the n-1 amino acid of the peptide to an N-terminal amino acid (referred to herein as the “n-1 NTAA”). Similarly, analysis of a peptide may begin from C-terminus towards the N-terminus with each round of cleavage from the C-terminus creating a new CTAA. Cleavage of the n CTAA converts the n-1 amino acid of the peptide to a C-terminal amino acid, referred to herein as an “n-1 CTAA”. Analyzing the peptide may also include determining a presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “analyte” generally refers to a substance that is of interest to be further identified, characterized, or measured. An analyte can be, in non-limiting examples, an ion, chemical, compound, small molecule, element, particle, metal, biomolecule, macromolecule, metabolite, lipid, carbohydrate, peptide or protein, nucleic acid molecule, organelle, or cell. An analyte may be naturally occurring or synthetic. The analyte may be a solid, semi-solid, liquid, semi-liquid, gas, or plasma. The analyte may be characterized qualitatively or quantitatively. A portion of an analyte may be analyzed. For example, an analyte may be a peptide and the constituent amino acids may be analyzed. The analyte may comprise a polymer, also referred to herein as “polymeric analyte”, which generally refers to an analyte of interest that comprises one or more monomers. A polymeric analyte can be, in non-limiting examples, a group of ions, chemicals, compounds, small molecules, elements, particles, metals, or a biomolecule, macromolecule, metabolite, lipid, carbohydrate, peptide or protein, nucleic acid molecule, organelle, or cell.

As used herein, the term “array” generally refers to a population of molecules that is attached to one or more solid supports such that the molecules at one address can be distinguished from molecules at other addresses. An array can include different molecules that are each located at different addresses on a solid support. Alternatively, an array can include separate solid supports each functioning as an address that bears a different molecule, wherein the different molecules can be identified according to the locations of the solid supports on a surface to which the solid supports are attached, or according to the locations of the solid supports in a liquid such as a fluid stream. The molecules of the array can be, for example, nucleic acids such as SNAPs, polypeptides, proteins, peptides, oligopeptides, enzymes, ligands, or receptors such as antibodies, functional fragments of antibodies or aptamers. The addresses of an array can optionally be optically observable, and, in some configurations, adjacent addresses can be optically distinguishable when detected using a method or apparatus set forth herein.

As used herein, the term “functionalized” refers to any material or substance that has been modified to include a functional group. A functionalized material or substance may be naturally or synthetically functionalized. For example, a polypeptide can be naturally functionalized with a phosphate group, oligosaccharide (e.g., glycosyl, glycosylphosphatidylinositol or phosphoglycosyl), nitrosyl, methyl, acetyl, lipid (e.g., glycosyl phosphatidylinositol, myristoyl or prenyl), ubiquitin or other naturally occurring post-translational modification. A functionalized material or substance may be functionalized for any given purpose, including altering chemical properties (e.g., altering hydrophobicity or changing surface charge density) or altering reactivity (e.g., capable of reacting with a moiety or reagent to form a covalent bond to the moiety or reagent).

As used herein, the term “click reaction,” “click chemistry,” or “bioorthogonal reaction” refers to single-step, thermodynamically favorable conjugation reaction utilizing biocompatible reagents. A click reaction may utilize no toxic or biologically incompatible reagents (e.g., acids, bases, heavy metals) or generate no toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary bioorthogonal and click reactions are described in detail in WO 2019/195633A1, which is herein incorporated by reference in its entirety. Exemplary click reactions may include metal-catalyzed azide-alkyne cycloaddition, strain-promoted azide-alkyne cycloaddition (SPAAC), strain-promoted azide-nitrone cycloaddition, strained alkene reactions, thiolene reaction, Diels-Alder reaction, inverse electron demand Diels-Alder reaction, [3+2] cycloaddition, [4+1] cycloaddition, nucleophilic substitution, dihydroxylation, thiolyne reaction, photoclick, nitrone dipole cycloaddition, norbornene cycloaddition, oxanobornadiene cycloaddition, tetrazine ligation, and tetrazole photoclick reactions. Exemplary functional groups or reactive handles utilized to perform click reactions (also referred to herein as “click chemistry moieties”) may include alkenes (e.g., linear alkenes or cyclic alkenes such as trans-cyclooctene (TCO)), alkynes (e.g., linear alkynes or cycloalkynes (e.g., cyclooctynes or derivatives thereof, e.g., aza-dimethoxycyclooctyne (DIMAC), symmetrical pyrrolocyclooctyne (SYPCO), pyrrolocyclooctyne (PYRROC), difluorocyclooctyne (DIFO), α,α-bis(trifluoromethyl) pyrrolocyclooctyne (TRIPCO), bicyclo[6.1.0] nonyne (BCN), dibenzocyclooctyne (DBCO), difluorinated cyclooctyne (DIFO), difluorobenzocyclooctyne (DIFBO), dibenzoazacyclo-octyne (DBACO), difluoro-aza-dibenzocyclooctyne (F₂-DIBAC), biaryl-azacyclooctynone (BARAC), difluorodimethoxydibenzocyclooctynol (FMDIBO), difluorodimethoxydibenzocyclooctynone (keto-FMDIBO), and 3,3,6,6-tetramethylthiacycloheptyne (TMTH)), TMTH-sulfoximine (TMTHSI), azides, epoxides, amines, thiols, nitrones, isonitriles, isocyanides, aziridines, activated esters, and tetrazines, triazoles, and combinations, variations, or derivatives thereof. The click chemistry moieties may be subjected to conditions sufficient to react the first click chemistry moiety to the second click chemistry moiety, e.g., provision of metal catalysts, appropriate solvents, pH, temperature, ionic concentration, or light/energy, for any useful duration of time.

As used herein, the terms “group” and “moiety” are intended to be synonymous when used in reference to the structure of a molecule. The terms refer to a component or part of the molecule. The terms do not necessarily denote the relative size of the component or part compared to the molecule, unless indicated otherwise. The terms do not necessarily denote the relative size of the component or part compared to any other component or part of the molecule, unless indicated otherwise. A group or moiety can contain one or more atoms.

As used herein, “primers” generally refer to nucleic acid molecules which can prime the synthesis of a nucleic acid molecule (e.g., DNA or RNA). A primer may be single stranded. A primer may comprise one or more recognition sites for a protein (e.g., a polymerizing enzyme, a restriction enzyme, a cleaving enzyme, a nuclease, etc.) to bind to the primer or a primer hybridized to a template strand. A primer may comprise DNA, RNA, or other nucleic acid analogs or noncanonical bases (e.g., spacer moieties, uracils, abasic sites). A primer may optionally comprise any number of functional sequences such as sequencing primer sequences (e.g., P5 or P7 sequences), sequencing primer-binding sequences, read sequences (e.g., R1 or R2 sequences), restriction sites, nuclease-recognition sites, abasic sites, cleavage sites, transposition sites (e.g., mosaic end sequences), a barcode sequence, a unique molecular identifier (UMI), etc.

“Amplification” or “amplifying” generally refers to a polynucleotide amplification reaction, namely, a population of polynucleotides that are replicated from one or more starting sequences. Amplifying may refer to a variety of amplification reactions, including but not limited to polymerase chain reaction (PCR), linear polymerase reactions, nucleic acid sequence-based amplification, rolling circle amplification and similar reactions. An amplification reaction may generate an amplicon. Amplification may be performed isothermally or by cycling of temperatures. Amplification or amplifying may refer to an increase in quantity of a measurable output, for example, signal amplification.

An “adapter” as referred to herein, generally refers to a short nucleic acid molecule (e.g., about 10 to about 100 base pairs in length). An adapter may comprise a short double-stranded DNA molecule. An adapter may be attached, e.g., via polymerization or ligation, to an end of a DNA fragments or amplicons. Adapters may comprise synthetic oligonucleotides, e.g., oligonucleotides that have nucleotide sequences which are at least partially complementary to each other. An adapter may have blunt ends, may have staggered ends (also referred to herein as a 3′ or 5′ “overhang sequence” or “sticky end”, or a blunt end and a staggered end. Adapters may be attached (e.g., via ligation) to fragments to provide an adapter-ligated fragment; the adapter-ligated fragment may serve as a starting point for subsequent manipulation e.g., for amplification or sequencing. An adapter may be functionalized, e.g., conjugated with a tag, probe, detectable label, affinity capture reagent (e.g., biotin or streptavidin). An adapter may comprise one of more functional sequences, e.g., a primer sequence (e.g., for sequencing, reading, or amplification), a restriction site, a recombination site, a transposition site, an abasic site, a barcode sequence, a translational control element, a splice site, an origin of replication, a promoter, an enhancer, a silencer, an insulator, and operator, a polyadenylation (polyA) site, or other functional sequence or a combination thereof.

The term “capture moiety” as used herein generally refers to a molecule that is configured to be coupled to another moiety or molecule. A capture moiety can be a biomolecule, e.g., a lipid, carbohydrate, sugar, amino acid, peptide or protein, nucleotide, nucleic acid molecule, metabolite, or a combination thereof (e.g., glycoproteins, lipoproteins, glycosaminoglycans, etc.). A capture moiety can be a small molecule, organic compound, inorganic compound, metal, polymer, ion, or other molecule or molecular compound. A capture moiety may comprise a macromolecule. A capture moiety may comprise an enzyme, antibody, antibody fragment, nanobody, aptamer, biotin, streptavidin, avidin, neutravidin, or analogs or derivatives thereof. A capture moiety may comprise more than one molecule, e.g., a dimer, trimer, tetramer, pentamer, hexamer, heptamer, octamer, etc. A capture moiety can be a solid substrate or part of a solid substrate, or the capture moiety can be separate from a substrate, e.g., in a fluidic medium (e.g., air, in a liquid solution). A capture moiety may have specificity to a binding partner or a plurality of binding partners. A capture moiety may be able to bind to one molecule or moiety (univalent), or a plurality of molecules or moieties (multivalent).

The term “translocating” and “translocation,” as used herein, generally refers to the movement of a molecule through a medium (e.g., a gas, a liquid, a solid, or a multiphase medium). Translocation of a molecule may occur spontaneously (e.g., through diffusion, Brownian motion, etc.). Alternatively, or in addition to, translocation of a molecule may occur with an application of force or pressure, e.g., using frictional force, tension force, a normal force, air resistance force, spring force, a temperature gradient, gravitational force, electrical force, magnetic force, acoustic force (e.g., acoustophoresis) etc. In some examples, translocation of a molecule may be achieved by application of pressure-driven flow or electrokinetic forces (e.g., electrophoretic force, electroosmotic force). Translocation may occur through a liquid or through a solid or semi-solid substrate (e.g., through a pore or gap) or adjacent or in proximity to the solid or semi-solid substrate.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R). References to these amino acids are also in the form of “[amino acid][residues/residues]” (e.g., lysine residue, lysine residues, leucine residue, leucine residues, etc.).

As used herein, the term “channel” refers to any passage, conduit, or pathway within a device or system that allows for the controlled movement or transport of fluid, gas, particle, or other material. The channel can vary in size, shape, and configuration, and may include straight, curved, branched, or interconnected segments to facilitate specific flow dynamics, mixing, separation, or reaction processes. A channel may be present in microfluidic systems, macroscopic devices, or other contexts where precise control of material transport is required, and may be fabricated from a variety of materials suitable for the intended environment or application. Channels may be coated or treated with a range of substances or surface modifications to alter their physical, chemical, or biological properties, to optimize flow dynamics or enhancing compatibility with different fluid, gas, particle, or any combination thereof.

As used herein, the term “nanopore” or “nanogap” generally refers to a pore, hole, aperture, gap, or channel of nanometer scale. The nanopore, nanogap, or nanochannel may be generated from an organic material, e.g., a pore-forming protein or a transmembrane protein. Such a protein may be naturally occurring, synthetic, or engineered. Examples of naturally occurring organic nanopores include wild-type aerolysin, alpha-hemolysin, mycobacterial porins (e.g., MspA porin), Phi29 connector channels, Fragaceatoxin C, Cytolysin A, Ferric hydroxyamate uptake component A, Curli specific gene G, outer membrane porin G, and viral DNA packaging motors. The nanopore, nanochannel, or nanogap may comprise an engineered variant (e.g., a nanopore that has been mutated at one or more amino acids, a metal-ion modified pore such as a pore in contact with or forming coordination interactions with copper or nickel ions or compounds thereof) of a naturally occurring nanopore. The nanopore, nanogap, or nanochannel may comprise an inorganic material. For example, solid-state nanopores may be made from dielectric materials such as a silicon compound (e.g., silicon nitride, silicon dioxide), an aluminum compound (e.g., aluminum oxide), a titanium compound (e.g., titanium oxide), a molybdenum compound (e.g., molybdenum disulfide), hafnium, graphene, etc. The nanopore, nanochannel, or nanogap may assume any useful form factor or geometry, e.g., gaps or channels within membranes, capillaries, etc., and may be generated using any suitable process, e.g., ion-beam sculpting, electron beam exposure. The nanopore, nanochannel, or nanogap may comprise an elastomeric material.

As used herein, the term “software” includes any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

While various embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

### EXAMPLES

### Example 1—Preparation of Modified Amino Acids for Nanopore Sequencing by Polymerization

Modified amino acids may be generated from peptide analytes, as described herein. For proof-of-concept experiments, modified amino acids of five different amino acids are synthesized chemically. The synthetic modified amino acids comprise a polymerizable molecule (a DNA molecule), a linker, and an amino acid that are covalently linked. The synthetic modified amino acids (referred to hereinafter as simply “modified amino acids”) are generated by coupling a polymerizable molecule (a DNA molecule) comprising a click chemistry moiety to an amino acid-linker complex comprising a second click chemistry moiety. Specifically, the polymerizable molecule comprises a DNA molecule comprising an Octadiynyl deoxyuridine (OctdU; Integrated DNA Technologies), which comprises an alkyne moiety. The DNA molecule comprises the sequence, CAGTTTTTGTGTGATGTGTTTTTxTTTTTGTGTGATGTGTGCAT (SEQ ID NO: 1), where “x” indicates the location of the OctdU.

The amino acid-linker complex is generated using a bifunctional linker, 1-(2-azidoethyl)-4-isothiocyanatobenzene, which comprises (1) a PITC moiety (amino acid reactive group) and (2) an azide moiety that is capable of reacting with the alkyne of the polymerizable molecule. The bifunctional linker is reacted with five different amino acids: Glu (E), Arg (R), Phe (F), Gly (G), and Pro (P) via the PITC moiety, thereby generating an amino acid-linker complex. The amino acid-linker complexes are then reacted with the DNA molecule comprising the OctdU using a click chemistry reaction, thereby generating a modified amino acid (comprising an amino acid-linker-polymerizable molecule conjugate). The modified amino acids are in the phenylthiocarbamoyl (PTC) form.

Next, the modified amino acids are prepared for sequencing via a polymerization-based nanopore sequencing system using an MspA nanopore. Briefly, sequencing adapters are attached to the modified amino acids. The sequencing adapters may comprise one or more blocking regions (e.g., a hairpin moiety) and/or one or more self-annealing regions.

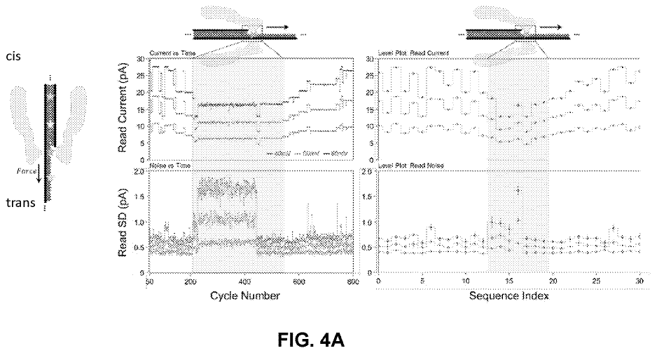

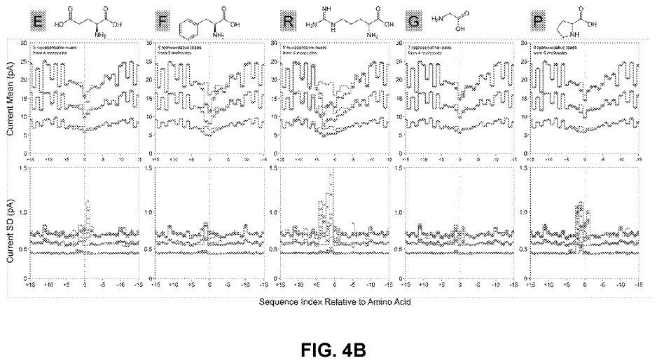

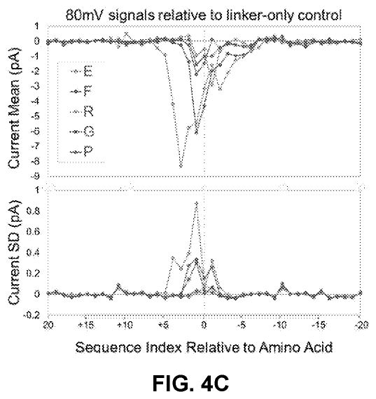

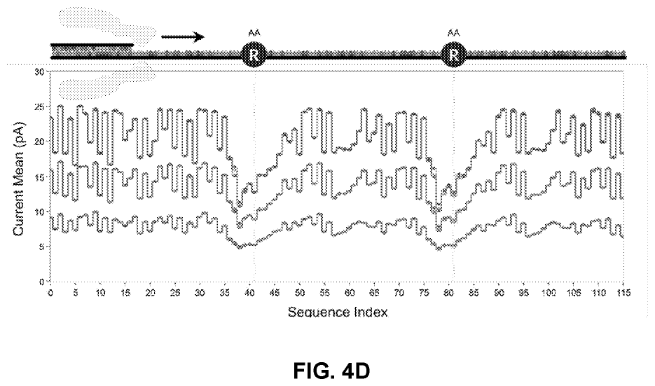

The modified amino acids are then sequenced using a polymerization-based nanopore sequencer, as described herein. FIG. 4A schematically shows (left) polymerization-based nanopore sequencing of a partially polymerized stacked plurality of modified amino acids and data (right) from sequencing of a PTC-Arginine (Arg, R) modified amino acid. In the schematic (left), cleaved amino acids are depicted as stars along a DNA (polymerizable molecule) backbone. The partially polymerized stacked plurality of amino acids is translocated in a first direction (e.g., cis to trans) through an aperture of the nanopore. The double-stranded region is arrested at the constriction region (narrowest portion) in a read position and the current is measured. The plots (right) show data generated from sequencing (measuring one or more signals from or adjacent to the nanopore of) the PTC-Arginine-containing modified amino acid. The top-left plot shows a measured current (“Read current (pA)”) as a function of time for three measured voltages (40 mV, 60 mV, and 80 mV). The gray-shaded region indicates the constriction region of the nanopore. A clear signal disruption (decrease) can be visualized at or adjacent to the position of the amino acid (PTC-Arginine) for all three measured voltages. The bottom-left plot shows the standard deviation of the current (“Read SD (pA)”) as a function of time, indicating a relatively large increase in the current standard deviation corresponding to the presence of the PTC-Arginine moiety present in the constriction region of the nanopore. The top-right plot shows the same measured current as a level plot, where each increment (dot) along the X-axis corresponds to a positional change (e.g., one nucleotide) of the DNA backbone through the nanopore for the three measured voltages. The gray-shaded region represents the measurement of the constriction region. When the modified amino acid is in the constriction zone, a current drop (decrease) is perceivable. The bottom-right plot shows the standard deviation of the current as a function of position, indicating a relatively large increase in the current standard deviation corresponding to the presence of the PTC-Arginine moiety present in the constriction region of the nanopore.

FIG. 4B shows current level plots of measured current mean as a function of position (top) and current standard deviation as a function of position (bottom) for five different modified amino acids comprising, from left to right, PTC-E, PTC-F, PTC-R, PTC-G, and PTC-P. The current level plots show 5 representative reads from 4 molecules; 9 representative reads from 6 molecules; 9 representative reads from 5 molecules; 7 representative reads from 4 molecules; and 9 representative reads from 8 molecules, respectively, for PTC-E, PTC-F, PTC-R, PTC-G, and PTC-P. Each plot shows the mean current or current standard deviation for three applied voltages. The gray line (non-dotted) shows the linker-only control, in which the DNA molecule comprises the OctdU but is not clicked to any amino acid-linker complex. The modified amino acids and linker-only control are translocated through the pore across thousands of sequencing (incorporate-read and re-sequencing) cycles. Both mean current signal and current standard deviation are distinct across the five different modified-amino acids, with the most pronounced current drop at position 0 for PTC-F at 80 mV (~47% drop) and PTC-R (~34% drop) and lower drops for PTC-P (~34% drop), PTC-G (~32%) and PTC-E (~30%). The current mean and standard deviation also respond to the different applied voltages. For instance, the mean current and current standard deviation do not change uniformly as a function of the applied voltage, but rather modulate in a position-specific manner adjacent to the amino acid and are influenced by the size and charge of the amino acid. Further, all five amino acids exhibit a high signal-to-noise ratio (SNR), where SNR is calculated as the magnitude of the current dip (magnitude of the average mean value at positions far away, i.e., greater than 8 nucleotides from the amino acid site-current mean at position 0) divided by the mean standard deviation at position 0. Calculated SNR values for PTC-F is ~15.7, PTC-R is ~12.9, PTC-G is ~11.1, PTC-P is ~9.5, and PTC-E is ~9.5.

In addition to mean current and current standard deviation, the coefficient of variation is informative. For example, at position 0, the incorporation site of the amino acid, the coefficient of variation shows distinct patterns for each amino acid, e.g., PTC-F shows a relatively high CV at 80 mV (~6%) arising from a large current dip; PTC-R shows a relatively high CV at 80 mV (~6%) with an extended dip region spanning positions +4 to −2, possibly due to a larger charged side chain interacting with the constriction region of the nanopore or adjacent sites; PTC-E shows moderate CV increases at position 0 (~5%) at 80 mV, and PTC-G and PTC-P show relatively similar CV profiles (~4%) at 80 mV, possibly due to the smaller side chain. For 40 mV, the CV is highest as the low voltage produces smaller mean currents while noise remains relatively constant. At 80 mV, the baseline CV is low but amino acid-specific dips generate larger relative noise increases. Each amino acid has a distinguishable CV fingerprint when considering all three voltages: PTC-F has the largest current dip with relatively low current standard deviation, creating a larger peak CV. PTC-R shows a broad elevated CV spanning ~5-6 positions; PTC-E shows a moderate, symmetric CV peak, PTC-G shows the smallest CV perturbation, and PTC-P is similar to PTC-G but with an asymmetric recovery. The maximum CV varies, depending on the position relative to the amino acid and the applied voltage. Across all five tested amino acids, the maximum CV observed is <10% (~9.6% for PTC-R at position 1 at 60 mV). These preliminary results suggest that unique CV profiles can serve as additional discriminating features for nanopore sequencing of modified amino acids. Further, the low CV values overall indicate a highly robust and reproducible method of sequencing modified amino acids.

FIG. 4C shows current level plots of measured current mean as a function of position (top) measured at 80 mV, and current standard deviation as a function of position (bottom) for the five different modified amino acids overlayed together. Both mean current signal and current standard deviation are background-subtracted from the mean current and standard deviation, respectively, of the linker-only control. Mean current signal and current standard deviation are distinct across the five different modified-amino acids, suggesting that the current signal, current standard deviation, or both may be used to accurately distinguish or identify different amino acid types. Additional other metrics or measurements, such as a mean value (μ_{signal}), a median value, a range of values (e.g., max signal−min signal), a standard deviation or noise of the signal (σ_{signal}), a coefficient of variation (σ_{signal}/μ_{signal}×100), a signal-to-noise ratio (SNR=μ_{signal}/σ_{signal}), peak width, peak signal, full width half max, peak position, separation resolution, area-under-the-curve (AUC), or other value or combination of values, or combinations thereof, may also be useful in identifying or distinguishing the amino acid types. In some instances, the other metrics or measurements is a ratio of the current and the voltage, e.g., a conductance (current (I)/voltage (V)) or a change in conductance (e.g., slope of the current/voltage or dI/dV).

FIG. 4D shows a current level plot of measured current mean as a function of position for a stacked plurality of modified amino acids comprising two PTC-arginine moieties. The current mean is measured across three different applied voltages. A distinct drop in the mean current across all three measured voltages is perceived at or adjacent to the amino acid site and shows a similar profile for both amino acid positions, showing robustness and precision of the polymerase-based nanopore sequencing.

Altogether, the results indicate that polymerase-based nanopore sequencing may be a feasible and robust method of identifying amino acid types comprised by modified amino acids or stacked pluralities of modified amino acids.

Example Embodiments. The present disclosure provides example embodiments. Among the provided embodiments are:

Embodiment 1. A method for processing a modified amino acid, comprising:
(a) providing said modified amino acid and a plurality of monomers, wherein said modified amino acid comprises a polymerizable molecule;
(b) incorporating a monomer of said plurality of monomers into or adjacent to said polymerizable molecule, thereby generating a partially polymerized modified amino acid; and
(c) translocating said partially polymerized modified amino acid or a portion thereof through a nanopore.

Embodiment 2. The method of embodiment 1, further comprising (d), measuring a signal of or adjacent to said nanopore, thereby generating a measured signal.

Embodiment 3. The method of embodiment 2, wherein (d) occurs during (c).

Embodiment 4. The method of embodiment 2 or 3, further comprising, (e) using said measured signal or a metric or characteristic thereof to determine an amino acid type of said modified amino acid.

Embodiment 5. The method of any one of embodiments 2-4, wherein said measured signal is a current signal under an applied voltage.

Embodiment 6. The method of embodiment 5, further comprising, measuring a plurality of signals of or adjacent to said nanopore, thereby generating a plurality of measured signals.

Embodiment 7. The method of embodiment 6, wherein said plurality of measured signals is a plurality of current signals under a plurality of applied voltages or a time-varying voltage.

Embodiment 8. The method of embodiment 6 or 7, further comprising, using said plurality of measured signals or a metric or characteristic thereof to identify an amino acid type of said modified amino acid.

Embodiment 9. The method of any preceding embodiment, wherein (c) occurs in a cis-to-trans direction.

Embodiment 10. The method of embodiment 9, wherein (c) is performed by applying an electric potential to said nanopore.

Embodiment 11. The method of embodiment 10, further comprising, reversing said electric potential, thereby translocating said partially polymerized modified amino acid in a trans-to-cis direction.

Embodiment 12. The method of embodiment 11, further comprising, repeating (b)-(c) and said reversing.

Embodiment 13. The method of embodiment 12, further comprising, (f) measuring an additional plurality of signals of or adjacent to said nanopore during said repeating.

Embodiment 14. The method of embodiment 13, further comprising, using said additional plurality of signals or a metric or characteristic thereof to identify an amino acid type of said modified amino acid.

Embodiment 15. The method of embodiment 4, 8, or 14, wherein said metric or characteristic thereof is a mean value (signal), a median value, a range of values (e.g., max signal-min signal), a standard deviation or noise of the signal (signal), a coefficient of variation (signal/signal×100), a signal-to-noise ratio (signal/signal), peak width, peak signal, full width half max, peak position, separation resolution, area-under-the-curve (AUC), or combination thereof.

Embodiment 16. The method of embodiment 4, 8, or 14, wherein said metric or characteristic thereof is a conductance or a conductance rate of change.

Embodiment 17. The method of any preceding embodiment, wherein said polymerizable molecule comprises a nucleic acid molecule.

Embodiment 18. The method of embodiment 17, wherein said nucleic acid molecule is at least partially single-stranded.

Embodiment 19. The method of embodiment 17 or 18, wherein, prior to (b), said nucleic acid molecule comprises a double-stranded priming region.

Embodiment 20. The method of embodiment 19, wherein said plurality of monomers comprises a plurality of nucleotides, and wherein said monomer is incorporated adjacent to said double-stranded priming region.

Embodiment 21. The method of any one of embodiments 17-20, wherein (b) is performed using a polymerizing enzyme.

Embodiment 22. The method of embodiment 21, wherein said polymerizing enzyme is a polymerase.

Embodiment 23. The method of embodiment 22, wherein said monomer is a nucleotide or modified nucleotide, and wherein, during (b), said polymerase incorporates a single nucleotide or modified nucleotide to said nucleic acid molecule.

Embodiment 24. The method of any one of embodiments 17-23, wherein said nucleic acid molecule comprises an error correction barcode sequence.

Embodiment 25. The method of embodiment 24, wherein said error correction barcode sequence comprises a Hamming code, a Levenshtein code, or a Reed-Solomon code.

Embodiment 26. The method of any preceding embodiment, wherein said modified amino acid is comprised by a stacked plurality of modified amino acids.

Embodiment 27. The method of embodiment 26, wherein said stacked plurality of modified amino acids comprises a first end and a second end, wherein said first end or said second end comprises a blocking region, wherein said blocking region prevents said stacked plurality of modified amino acids from escaping said nanopore.

Embodiment 28. The method of embodiment 27, wherein said first end and said second end comprise said blocking region.

Embodiment 29. The method of embodiment 27 or 28, wherein said blocking region comprises a hairpin structure, a G-quadruplex, or a protein.

Embodiment 30. The method of any preceding embodiment, wherein (b) and (c) are performed in a single reaction mixture.

Embodiment 31. The method of any preceding embodiment, further comprising, generating the modified amino acid, wherein the generating comprises (I) providing a linker and said polymerizable molecule, (II) coupling the linker to (i) an amino acid of a peptide and (ii) the polymerizable molecule to generate an amino acid-linker complex, and (III) cleaving the amino acid, thereby generating the modified amino acid, wherein the modified amino acid comprises a cleaved amino acid, the linker, and the polymerizable molecule.

Embodiment 32. The method of embodiment 31, wherein said coupling of said linker to said polymerizable molecule occurs prior to (I).

Embodiment 33. The method of any one of embodiments 2-32, further comprising, re-sequencing said modified amino acid, wherein said re-sequencing comprises removing said monomer from said partially polymerized modified amino acid, thereby re-generating said modified amino acid and repeating (b)-(c).

Embodiment 34. The method of embodiment 33, wherein said re-sequencing further comprises measuring an additional signal of or adjacent to said nanopore, thereby generating an additional measured signal.

Embodiment 35. The method of embodiment 34, further comprising, using said measured signal and said additional measured signal to identify an amino acid type, wherein an identification accuracy of said amino acid type is higher using said measured signal and said additional measured signal as compared to an identification accuracy using only said measured signal or only said additional measured signal.

Embodiment 36. The method of any one of embodiments 33-35, further comprising, performing said re-sequencing at least 5 times, wherein a consensus accuracy of said re-sequencing for a given amino acid type exceeds 80% for at least 10 distinct amino acid types.

Embodiment 37. The method of any preceding embodiment, wherein said nanopore is an MspA nanopore or variant thereof.

Embodiment 38. The method of any preceding embodiment, wherein (a) further comprises providing a plurality of modified amino acids including said modified amino acid, and wherein the method further comprises performing differential labeling of said plurality of modified amino acids to confirm the identity of at least one modified amino acid of said plurality of modified amino acids.

Embodiment 39. A method for identifying a stacked plurality of modified amino acids, comprising:
(a) providing said stacked plurality of modified amino acids, wherein said stacked plurality of modified amino acids comprises a plurality of modified amino acids comprising a plurality of polymerizable molecules, wherein said plurality of polymerizable molecules are coupled to one another in a linear fashion;
(b) contacting said stacked plurality of modified amino acids with a plurality of monomers;
(c) incorporating a monomer of said plurality of monomers into or adjacent to a polymerizable molecule of said plurality of polymerizable molecules, thereby generating a partially polymerized stacked plurality of modified amino acids;
(d) translocating, in a first direction, said partially polymerized stacked plurality of modified amino acids or a portion thereof through a nanopore;
(e) translocating, in a second direction, said partially polymerized stacked plurality of modified amino acids or a portion thereof through said nanopore; and
(f) repeating (c)-(e) on said partially polymerized stacked plurality of modified amino acids.

Embodiment 40. The method of embodiment 39, further comprising, during (d) or (e), measuring a signal from or adjacent to said nanopore, thereby generating a measured signal.

Embodiment 41. The method of embodiment 40, further comprising, using said measured signal or a metric or characteristic thereof to determine an amino acid type of a modified amino acid of said stacked plurality of modified amino acids.

Embodiment 42. The method of embodiment 40 or 41, wherein said measured signal is a current signal under an applied voltage.

Embodiment 43. The method of embodiment 42, further comprising, measuring a plurality of signals of or adjacent to said nanopore, thereby generating a plurality of measured signals.

Embodiment 44. The method of embodiment 43, wherein said plurality of measured signals is a plurality of current signals under a plurality of applied voltages or a time-varying voltage.

Embodiment 45. The method of any one of embodiments 39-44, further comprising, measuring an additional plurality of signals of or adjacent to said nanopore during (f).

Embodiment 46. The method of embodiment 45, further comprising, using said additional plurality of signals or a metric or characteristic thereof to identify an amino acid type of said stacked plurality of modified amino acids.

Embodiment 47. The method of embodiment 43, further comprising, using said plurality of measured signals or a metric or characteristic thereof to identify one or more amino acid types of said plurality of modified amino acids.

Embodiment 48. The method of embodiment 41 or 47, wherein said metric or characteristic thereof is a mean value (signal), a median value, a range of values (e.g., max signal-min signal), a standard deviation or noise of the signal (signal), a coefficient of variation (signal/signal×100), a signal-to-noise ratio (signal/signal), peak width, peak signal, full width half max, peak position, separation resolution, area-under-the-curve (AUC), or combination thereof.

Embodiment 49. The method of embodiment 41 or 47, wherein said metric or characteristic thereof is a conductance or a conductance rate of change.

Embodiment 50. The method of any one of embodiments 39-47, wherein said plurality of polymerizable molecules comprises a plurality of nucleic acid molecules.

Embodiment 51. The method of embodiment 50, wherein said stacked plurality of modified amino acids comprises a double-stranded priming region.

Embodiment 52. The method of embodiment 51, wherein said plurality of monomers comprises a plurality of nucleotides, and wherein said monomer is incorporated adjacent to said double-stranded priming region.

Embodiment 53. The method of any one of embodiments 50-52, wherein (c) is performed using a polymerizing enzyme.

Embodiment 54. The method of embodiment 53, wherein said polymerizing enzyme is a polymerase.

Embodiment 55. The method of embodiment 54, wherein said monomer is a nucleotide or a modified nucleotide, and wherein, during (c), said polymerase incorporates a single nucleotide or modified nucleotide to said polymerizable molecule.

Embodiment 56. The method of any one of embodiments 50-55, wherein said plurality of nucleic acid molecules comprises an error correction barcode sequence.

Embodiment 57. The method of embodiment 56, wherein said error correction barcode sequence comprises a Hamming code, a Levenshtein code, or a Reed-Solomon code.

Embodiment 58. The method of any one of embodiments 39-55, wherein a first end and a second end of said stacked plurality of modified amino acids comprises a blocking region.

Embodiment 59. The method of embodiment 58, wherein said blocking region comprises a hairpin structure, a G-quadruplex or a protein.

Embodiment 60. The method of any one of embodiments 39-59, wherein (d) occurs in a cis-to-trans direction.

Embodiment 61. The method of embodiment 60, wherein (d) is performed by applying an electric potential to said nanopore.

Embodiment 62. The method of embodiment 61, wherein (e) comprises reversing said electric potential, thereby translocating said partially polymerized stacked plurality of modified amino acids in a trans-to-cis direction.

Embodiment 63. The method of any one of embodiments 39-62, wherein (b)-(f) are performed in a single reaction mixture.

Embodiment 64. The method of any one of embodiments 39-63, further comprising, generating the stacked plurality of modified amino acids.

Embodiment 65. The method of any one of embodiments 40-64, further comprising, re-sequencing said stacked plurality of modified amino acids, wherein said re-sequencing comprises removing monomers from said partially polymerized stacked plurality of modified amino acids, thereby re-generating said stacked plurality of modified amino acids of (a), and repeating (b)-(e).

Embodiment 66. The method of embodiment 65, wherein said re-sequencing further comprises measuring an additional signal of or adjacent to said nanopore, thereby generating an additional measured signal.

Embodiment 67. The method of embodiment 66, further comprising, using said measured signal and said additional measured signal to identify an amino acid type of said stacked plurality of modified amino acids, wherein an identification accuracy of said amino acid type is higher using said measured signal and said additional measured signal as compared to an identification accuracy using only said measured signal or only said additional measured signal.

Embodiment 68. The method of any one of embodiments 65-67, further comprising, performing said re-sequencing at least 5 times, wherein a consensus accuracy of said re-sequencing for a given amino acid type exceeds 80% for at least 10 distinct amino acid types.

Embodiment 69. The method of any one of embodiments 39-68, wherein said nanopore is an MspA nanopore or variant thereof.

Embodiment 70. The method of any one of embodiments 39-69, further comprising performing differential labeling of said stacked plurality of modified amino acids to confirm the identity of at least one modified amino acid of said plurality of modified amino acids.

Embodiment 71. A method for processing a modified amino acid, comprising:
(a) in an incorporate position, contacting said modified amino acid with a plurality of monomers and a polymerizing enzyme, wherein said modified amino acid comprises a polymerizable molecule and wherein, in said incorporate position, said polymerizing enzyme incorporates n monomers into or adjacent to said polymerizable molecule of said modified amino acid, thereby generating a partially polymerized modified amino acid, wherein n is an integer between 0 and 10;
(b) translocating said partially polymerized modified amino acid or a portion thereof through a nanopore to a read position; and
(c) in said read position, measuring a signal from or adjacent to said nanopore.

Embodiment 72. The method of embodiment 71, further comprising, repeating (a) and (b).

Embodiment 73. The method of embodiment 72, wherein, given a number of repeated cycles of (a) and (b), said polymerizing enzyme incorporates n monomers according to a Poisson distribution.

Embodiment 74. The method of embodiment 73, wherein an average value of n across said repeated cycles is less than 1.

Embodiment 75. The method of any one of embodiments 71-74, wherein said polymerizable molecule comprises a nucleic acid molecule.

Embodiment 76. The method of embodiment 75, wherein said nucleic acid molecule comprises an error correction barcode sequence.

Embodiment 77. The method of embodiment 76, wherein said error correction barcode sequence comprises a Hamming code, a Levenshtein code, or a Reed-Solomon code.

Embodiment 78. The method of any one of embodiments 71-77, further comprising, prior to (a), providing a plurality of modified amino acids including said modified amino acid, and wherein the method further comprises performing differential labeling of said plurality of modified amino acids to confirm the identity of at least one modified amino acid of said plurality of modified amino acids.

Embodiment 79. A method for identifying an amino acid type comprised by a modified amino acid, comprising:
(a) translocating, in a first direction, said modified amino acid through a nanopore, wherein said modified amino acid comprises a polymerizable molecule;
(b) during (a), measuring a signal from or adjacent to said nanopore, thereby generating a measured signal;
(c) translocating said modified amino acid in a second direction;
(d) repeating (a)-(c), thereby generating a plurality of measured signals, wherein said plurality of signals, at a given position, have a coefficient of variation of less than about 15%; and
(e) using said plurality of measured signals to identify said amino acid type.

Embodiment 80. The method of embodiment 79, wherein said coefficient of variation is less than about 10%.

Embodiment 81. The method of embodiment 79, wherein said coefficient of variation is less than about 5%.

Embodiment 82. The method of any one of embodiments 79-81, wherein said polymerizable molecule comprises a nucleic acid molecule.

Embodiment 83. The method of embodiment 82, wherein said nucleic acid molecule comprises an error correction barcode sequence.

Embodiment 84. The method of embodiment 83, wherein said error correction barcode sequence comprises a Hamming code, a Levenshtein code, or a Reed-Solomon code.

Embodiment 85. The method of any one of embodiments 79-84, further comprising, prior to (a), providing a plurality of modified amino acids including said modified amino acid, and wherein the method further comprises performing differential labeling of said plurality of modified amino acids to confirm the identity of at least one modified amino acid of said plurality of modified amino acids.

Embodiment 86. A method for identifying one or more amino acid types comprised by a plurality of identical modified amino acids, comprising:
(a) translocating said plurality of identical modified amino acids through one or more nanopores; and
(b) during (a), measuring a signal from or adjacent to said one or more nanopores, thereby generating a plurality of measured signals corresponding to each modified amino acid of said plurality of identical modified amino acids, wherein said plurality of measured signals, at a given position, have a coefficient of variation of less than about 15%; and
(c) using said plurality of measured signals to identify said one or more amino acid types.

Embodiment 87. The method of embodiment 86, wherein said coefficient of variation is less than about 10%.

Embodiment 88. The method of embodiment 86, wherein said coefficient of variation is less than about 5%.

Embodiment 89. The method of any one of embodiments 86-88, wherein each modified amino acid of said plurality of identical modified amino acids comprises a nucleic acid molecule.

Embodiment 90. The method of embodiment 89, wherein said nucleic acid molecule comprises an error correction barcode sequence.

Embodiment 91. The method of embodiment 90, wherein said error correction barcode sequence comprises a Hamming code, a Levenshtein code, or a Reed-Solomon code.

Embodiment 92. The method of any one of embodiments 86-91, further comprising performing differential labeling of said plurality of identical modified amino acids to confirm the identity of said one or more amino acid types comprised by said plurality of identical modified amino acids.

Embodiment 93. A method for improving accuracy of identification of a modified amino acid, comprising:
(a) providing said modified amino acid and a plurality of monomers, wherein said modified amino acid comprises a polymerizable molecule;
(b) translocating said modified amino acid or a portion thereof through a nanopore;
(c) measuring a plurality of signals under a plurality of applied voltages at or adjacent to said nanopore, thereby generating a plurality of measured signals; and
(d) using said plurality of measured signals to identify an amino acid type of said modified amino acid, wherein an accuracy of identification of said amino acid type is higher using said plurality of measured signals from said plurality of applied voltages as compared to an accuracy of identification using measured signals from a single applied voltage.

Embodiment 94. The method of embodiment 93, wherein said plurality of applied voltages is a time-varying voltage.

Embodiment 95. The method of embodiment 93, wherein (c) occurs while said modified amino acid is positioned in a constriction region of said nanopore.

Embodiment 96. The method of embodiment 93, wherein said plurality of applied voltages is in a range of about 20 mV to about 120 mV.

Embodiment 97. The method of any one of embodiments 93-96, wherein said polymerizable molecule comprises a nucleic acid molecule.

Embodiment 98. The method of embodiment 97, wherein said nucleic acid molecule comprises an error correction barcode sequence.

Embodiment 99. The method of embodiment 98, wherein said error correction barcode sequence comprises a Hamming code, a Levenshtein code, or a Reed-Solomon code.

Embodiment 100. The method of any one of embodiments 93-99, further comprising, prior to (a), providing a plurality of modified amino acids including said modified amino acid, and wherein the method further comprises performing differential labeling of said plurality of modified amino acids to confirm the identity of at least one modified amino acid of said plurality of modified amino acids.

Embodiment 101. A method for processing a modified amino acid, comprising:
(a) providing said modified amino acid and a plurality of monomers, wherein said modified amino acid comprises a nucleic acid molecule;
(b) coupling a plurality of primers to said nucleic acid molecule, thereby generating a plurality of double-stranded regions along said nucleic acid molecule; and
(c) translocating said modified amino acid from (b), or a portion thereof, through a nanopore, wherein said translocating is performed in absence of a motor protein.

Embodiment 102. The method of embodiment 101, wherein a double-stranded region of said plurality of double-stranded regions is configured to position a portion of said modified amino acid comprising an amino acid in a constriction region of said nanopore.

Embodiment 103. The method of embodiment 101 or 102, wherein said nucleic acid molecule comprises an error correction barcode sequence.

Embodiment 104. The method of embodiment 103, wherein said error correction barcode sequence comprises a Hamming code, a Levenshtein code, or a Reed-Solomon code.

Embodiment 105. The method of any one of embodiments 101-104, further comprising, prior to (a), providing a plurality of modified amino acids including said modified amino acid, and wherein the method further comprises performing differential labeling of said plurality of modified amino acids to confirm the identity of at least one modified amino acid of said plurality of modified amino acids.

Embodiment 106. A system configured to process a modified amino acid, comprising:
said modified amino acid, wherein said modified amino acid comprises a polymerizable molecule, wherein said modified amino acid is derived from a peptide analyte;
a plurality of monomers;
a polymerizing enzyme configured to incorporate a monomer of said plurality of monomers into or adjacent to said polymerizable molecule; and
a nanopore.

Embodiment 107. The system of embodiment 106, further comprising a voltage source configured to apply a plurality of voltages across said nanopore.

Embodiment 108. The system of embodiment 106, further comprising a measurement circuit configured to measure an ionic current through said nanopore.

Embodiment 109. The system of embodiment 108, wherein a sampling rate of said ionic current through said nanopore is at least 1 kHz.

Embodiment 110. The system of embodiment 106, further comprising, a processor configured to determine an amino acid type comprised by said modified amino acid.

Embodiment 111. The system of any one of embodiments 106-110, wherein said polymerizable molecule comprises a nucleic acid molecule.

Embodiment 112. The system of embodiment 111, wherein said nucleic acid molecule comprises an error correction barcode sequence.

Embodiment 113. The system of embodiment 112, wherein said error correction barcode sequence comprises a Hamming code, a Levenshtein code, or a Reed-Solomon code.

What is claimed is:

1. A method for measuring a signal of a stacked plurality of modified amino acids, comprising:
(a) providing said stacked plurality of modified amino acids, wherein said stacked plurality of modified amino acids comprises a plurality of modified amino acids comprising a plurality of nucleic acid molecules coupled thereto, wherein said plurality of nucleic acid molecules is coupled to one another in a linear fashion to produce a linear nucleic acid molecule, wherein said linear nucleic acid molecule comprises a double stranded priming region;
(b) contacting said stacked plurality of modified amino acids with a plurality of nucleotides and a polymerizing enzyme under conditions sufficient to perform a primer extension reaction of said double stranded priming region, thereby generating a partially polymerized stacked plurality of modified amino acids;
(c) translocating, in a first direction, said partially polymerized stacked plurality of modified amino acids or a portion thereof through a nanopore;
(d) translocating, in a second direction, said partially polymerized stacked plurality of modified amino acids or a portion thereof through said nanopore; and
(e) repeating (b)-(d) on said partially polymerized stacked plurality of modified amino acids;
wherein, during (d), said signal is measured from or adjacent to said nanopore, thereby generating a measured signal corresponding to an identity of a modified amino acid of said plurality of modified amino acids.

2. The method of claim 1, further comprising, using said measured signal or a metric or characteristic thereof to determine said identity of said modified amino acid.

3. The method of claim 2, wherein said metric or said characteristic of said signal measured in (d) comprises a mean value (μ_{signal}) and a signal standard deviation (σ_{signal}).

4. The method of claim 1, wherein said measured signal is a current signal under an applied voltage.

5. The method of claim 1, further comprising, measuring a plurality of signals in (d) corresponding to one or more modified amino acids of said plurality of modified amino acids, thereby generating a plurality of measured signals.

6. The method of claim 5, wherein said plurality of measured signals is a plurality of current signals under a plurality of applied voltages.

7. The method of claim 5, further comprising, using said plurality of measured signals or a metric or characteristic thereof to identify two or more modified amino acids of said plurality of modified amino acids.

8. The method of claim 7, wherein said metric or said characteristic of said plurality of measured signals comprises a mean value (μ_{signal}) and a standard deviation of the signal (σ_{signal}).

9. The method of claim 7, wherein said metric or said characteristic thereof is a conductance or a conductance rate of change.

10. The method of claim 1, wherein said double-stranded priming region comprises a primer.

11. The method of claim 10, further comprising, in (a), contacting said stacked plurality of modified amino acids with said primer, thereby generating said double stranded priming region.

12. The method of claim 1, wherein said polymerizing enzyme is a polymerase.

13. The method of claim 1, wherein during (b), said polymerizing enzyme incorporates a single nucleotide of said plurality of nucleotides into said linear nucleic acid molecule.

14. The method of claim 1, wherein said plurality of nucleic acid molecules comprises an error correction barcode sequence.

15. The method of claim 14, wherein said error correction barcode sequence comprises a Hamming code, a Levenshtein code, or a Reed-Solomon code.

16. The method of claim 1, wherein said stacked plurality of modified amino acids comprises a blocking region coupled to a first end and a second end of said linear nucleic acid molecule.

17. The method of claim 16, wherein said blocking region comprises a hairpin structure, a G-quadruplex, or a protein.

18. The method of claim 17, wherein said blocking region comprises a hairpin structure comprising said double stranded priming region.

19. The method of claim 1, wherein (c) occurs in a cis-to-trans direction.

20. The method of claim 19, wherein (c) is performed by applying an electric potential to said nanopore.

21. The method of claim 20, wherein (d) comprises reversing said electric potential, thereby translocating said partially polymerized stacked plurality of modified amino acids in a trans-to-cis direction.

22. The method of claim 1, wherein (b)-(d) are performed in a single reaction mixture.

23. The method of claim 1, further comprising, generating said stacked plurality of modified amino acids.

24. The method of claim 1, further comprising, re-sequencing said stacked plurality of modified amino acids, wherein said re-sequencing comprises removing nucleotides from said partially polymerized stacked plurality of modified amino acids, thereby re-generating said stacked plurality of modified amino acids of (a), and repeating (b)-(d).

25. The method of claim 24, wherein said re-sequencing further comprises measuring an additional signal of or adjacent to said nanopore, thereby generating an additional measured signal.

26. The method of claim 25, further comprising, using said measured signal and said additional measured signal to identify an amino acid type of said stacked plurality of modified amino acids, wherein an identification accuracy of said amino acid type is higher using said measured signal and said additional measured signal as compared to an identification accuracy using only said measured signal or only said additional measured signal.

27. The method of claim 24, further comprising, performing said re-sequencing at least 5 times, wherein a consensus accuracy of said re-sequencing for a given amino acid type exceeds 80% for at least 10 distinct amino acid types.

28. The method of claim 1, wherein said nanopore is an MspA nanopore or variant thereof.

29. The method of claim 1, further comprising performing differential labeling of said stacked plurality of modified amino acids.


SEQUENCE LISTING (1 sequence)

SEQ ID NO: 1 - DNA, 44 bp, synthetic construct
  source 1..44: mol_type = other DNA
  modified_base 24: mod_base = OTHER; note = Octadiynyl deoxyuridine
cagtttttgtgtgatgtgtttttntttttgtgtgatgtgtgcat